SEQUENCE LISTING (23 sequences)

SEQ ID NO: 1 - protein, 21 aa, Homo sapiens
  source 1..21: note = Ciltacabtagene autoleucel CAR CD8alpha signal peptide, CD8alpha SP amino acid sequence
MALPVTALLLPLALLLHAARP

SEQ ID NO: 2 - protein, 119 aa, Lama glama
  source 1..119: note = Ciltacabtagene autoleucel CAR BCMA binding domain, VHH1 amino acid sequence
QVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSS

SEQ ID NO: 3 - protein, 5 aa, synthetic construct
  source 1..5: note = Ciltacabtagene autoleucel CAR BCMA binding domain, G4S linker amino acid sequence
GGGGS

SEQ ID NO: 4 - protein, 118 aa, Lama glama
  source 1..118: note = Ciltacabtagene autoleucel CAR BCMA binding domain, VHH2 amino acid sequence
EVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSS

SEQ ID NO: 5 - protein, 45 aa, Homo sapiens
  source 1..45: note = Ciltacabtagene autoleucel CAR CD8alpha hinge amino acid sequence
TTTPAPRPPTPAPTIASQPLSLRPEACRPAAGGAVHTRGLDFACD

SEQ ID NO: 6 - protein, 24 aa, Homo sapiens
  source 1..24: note = Ciltacabtagene autoleucel CAR CD8alpha transmembrane amino acid sequence
IYIWAPLAGTCGVLLLSLVITLYC

SEQ ID NO: 7 - protein, 42 aa, Homo sapiens
  source 1..42: note = Ciltacabtagene autoleucel CAR CD137 Cytoplasmic amino acid sequence
KRGRKKLLYIFKQPFMRPVQTTQEEDGCSCRFPEEEEGGCEL

SEQ ID NO: 8 - protein, 112 aa, Homo sapiens
  source 1..112: note = Ciltacabtagene autoleucel CAR CD3zeta Cytoplasmic amino acid sequence
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - DNA, 63 bp, Homo sapiens
  source 1..63: mol_type = other DNA; note = Ciltacabtagene autoleucel CAR CD8alpha signal peptide CD8alpha SP nucleic acid sequence
atggctctgcccgtcaccgctctgctgctgcctctggctctgctgctgcacgctgctcgccct

SEQ ID NO: 10 - DNA, 357 bp, Lama glama
  source 1..357: mol_type = other DNA; note = Ciltacabtagene autoleucel CAR BCMA binding domain, VHH1 nucleic acid sequence
caggtcaaactggaagaatctggcggaggcctggtgcaggcaggacggagcctgcgcctgagctgcgcagcatccgagcacaccttcagctcccacgtgatgggctggtttcggcaggccccaggcaaggagagagagagcgtggccgtgatcggctggagggacatctccacatcttacgccgattccgtgaagggccggttcaccatcagccgggacaacgccaagaagacactgtatctgcagatgaacagcctgaagcccgaggacaccgccgtgtactattgcgcagcaaggagaatcgacgcagcagactttgattcctggggccagggcacccaggtgacagtgtctagc

SEQ ID NO: 11 - DNA, 15 bp, synthetic construct
  source 1..15: mol_type = other DNA; note = Ciltacabtagene autoleucel CAR BCMA binding domain, G4S linker nucleic acid sequence
ggaggaggaggatct

SEQ ID NO: 12 - DNA, 354 bp, Lama glama
  source 1..354: mol_type = other DNA; note = Ciltacabtagene autoleucel CAR BCMA binding domain, VHH2 nucleic acid sequence
gaggtgcagctggtggagagcggaggcggcctggtgcaggccggaggctctctgaggctgagctgtgcagcatccggaagaaccttcacaatgggctggtttaggcaggcaccaggaaaggagagggagttcgtggcagcaatcagcctgtcccctaccctggcctactatgccgagagcgtgaagggcaggtttaccatctcccgcgataacgccaagaatacagtggtgctgcagatgaactccctgaaacctgaggacacagccctgtactattgtgccgccgatcggaagagcgtgatgagcattagaccagactattgggggcagggaacacaggtgaccgtgagcagc

SEQ ID NO: 13 - DNA, 135 bp, Homo sapiens
  source 1..135: mol_type = other DNA; note = Ciltacabtagene autoleucel CAR CD8alpha hinge nucleic acid sequence
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 14 - DNA, 72 bp, Homo sapiens
  source 1..72: mol_type = other DNA; note = Ciltacabtagene autoleucel CAR CD8alpha transmembrane nucleic acid sequence
atctacatctgggcgcccttggccgggacttgtggggtccttctcctgtcactggttatcaccctttactgc

SEQ ID NO: 15 - DNA, 126 bp, Homo sapiens
  source 1..126: mol_type = other DNA; note = Ciltacabtagene autoleucel CAR CD137 Cytoplasmic nucleic acid sequence
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 16 - DNA, 339 bp, Homo sapiens
  source 1..339: mol_type = other DNA; note = Ciltacabtagene autoleucel CAR CD3zeta Cytoplasmic nucleic acid sequence
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 17 - protein, 488 aa, synthetic construct
  source 1..488: note = Ciltacabtagene autoleucel CAR amino acid sequence
MALPVTALLLPLALLLHAARPQVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSGGGGSEVQLVESGGGLVQAGGSLRLSCAASGRTFTMGWFRQAPGKEREFVAAISLSPTLAYYAESVKGRFTISRDNAKNTVVLQMNSLKPEDTALYYCAADRKSVMSIRPDYWGQGTQVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 5 aa, Lama glama
  source 1..5: note = Ciltacabtagene autoleucel CAR BCMA binding domain, VHH1 CDR1 amino acid sequence
SHVMG

SEQ ID NO: 19 - protein, 17 aa, Lama glama
  source 1..17: note = Ciltacabtagene autoleucel CAR BCMA binding domain, VHH1 CDR2 amino acid sequence
VIGWRDISTSYADSVKG

SEQ ID NO: 20 - protein, 11 aa, Lama glama
  source 1..11: note = Ciltacabtagene autoleucel CAR BCMA binding domain, VHH1 CDR3 amino acid sequence
ARRIDAADFDS

SEQ ID NO: 21 - protein, 5 aa, Lama glama
  source 1..5: note = Ciltacabtagene autoleucel CAR BCMA binding domain, VHH2 CDR1 amino acid sequence
TFTMG

SEQ ID NO: 22 - protein, 17 aa, Lama glama
  source 1..17: note = Ciltacabtagene autoleucel CAR BCMA binding domain, VHH2 CDR2 amino acid sequence
AISLSPTLAYYAESVKG

SEQ ID NO: 23 - protein, 13 aa, Lama glama
  source 1..13: note = Ciltacabtagene autoleucel CAR BCMA binding domain, VHH2 CDR3 amino acid sequence
ADRKSVMSIRPDY

(19) United States
(12) Patent Application Publication
(10) Pub. No.: US 2025/0269024 A1
(43) Pub. Date: Aug. 28, 2025

(54) BCMA-TARGETED CAR-T CELL THERAPY FOR MULTIPLE MYELOMA

(71) Applicants: LEGEND BIOTECH USA INC., Somerset, NJ (US); JANSSEN BIOTECH, INC., Horsham, PA (US)

(72) Inventors: Muhammad Shoaib AKRAM, Monroe, NJ (US); Nikoletta LENDVAI, West Windsor, NJ (US); Yogesh JETHAVA, Carmel, IN (US); Jordan Mark SCHECTER, Livingston, NJ (US); Kevin DE BRAGANCA, Chappaqua, NY (US)

(73) Assignees: LEGEND BIOTECH USA INC., Somerset, NJ (US); JANSSEN BIOTECH, INC., Horsham, PA (US)

(21) Appl. No.: 19/045,247

(22) Filed: Feb. 4, 2025

Related U.S. Application Data
(60) Provisional application No. 63/549,995, filed on Feb. 5, 2024.

(51) Int. Cl. A61K 40/42; A61K 40/11; A61K 40/31; A61P 35/00; C07K 14/725; C07K 16/28

(52) U.S. Cl. CPC A61K 40/4215; A61K 40/11; A61K 40/31; A61P 35/00; C07K 14/7051; C07K 16/2878; C07K 2317/569

(57) ABSTRACT

Provided herein are methods of treating a subject who has multiple myeloma and has received an initial therapy, including a stem cell transplantation. Infusions of chimeric antigen receptor (CAR)-T cells comprising a BCMA CAR comprising a polypeptide are administered to the subject. In certain embodiments, the dose of CAR-T cells administered to the subject is from 1.0×10⁵ to 5.0×10⁶ of CAR-T cells per kilogram of the subject's mass. The method of treatment is effective in obtaining and maintaining minimal residual disease negativity status, as well as other beneficial clinical outcomes related to efficacy and safety.

### CROSS-REFERENCE TO RELATED APPLICATIONS

This application claims the benefit of U.S. Provisional Patent Application No. 63/549,995, filed Feb. 5, 2024, the disclosure of which is incorporated by reference herein in its entirety.

### SEQUENCE LISTING

This application contains an electronic Sequence Listing which has been submitted in XML file format via Patent Center, the entire content of which is incorporated by reference herein in its entirety. The Sequence Listing XML file submitted via Patent Center is entitled “14651-062-999_SEQ_LISTING.xml”, was created on Jan. 24, 2025, and is 28,405 bytes in size.

### 1. BACKGROUND

Multiple myeloma is a neoplasm of plasma cells that is aggressive. Multiple myeloma is considered to be a B-cell neoplasm that proliferates uncontrollably in the bone marrow. Symptoms include one or more of hypercalcemia, renal insufficiency, anemia, bony lesions, bacterial infections, hyperviscosity and amyloidosis. Multiple myeloma is still considered to be an incurable disease, despite availability of new therapies that include proteasome inhibitors, immunomodulatory drugs, and monoclonal antibodies that have significantly improved patient outcomes. Because most patients will either relapse or become refractory to treatment, there is an ongoing need for new therapies or new treatment regimens for multiple myeloma.

### 2. SUMMARY OF THE DISCLOSURE

In one aspect is provided a method of treating a subject with multiple myeloma, wherein the subject has not achieved a complete response after receiving an initial therapy comprising a stem cell transplantation, the method comprising administering to the subject a dose of T cells comprising a chimeric antigen receptor (CAR) comprising:
(a) an extracellular antigen binding domain capable of specifically binding to an epitope of B-cell maturation antigen (BCMA), wherein the extracellular antigen binding domain comprises a first VHH domain and a second VHH domain, and wherein the first VHH domain comprising a CDR1, a CDR2, and a CDR3 as set forth in the VHH domain comprising the amino acid sequence of SEQ ID NO: 2, and the second VHH domain comprising a CDR1, a CDR2, and a CDR3 as set forth in the VHH domain comprising the amino acid sequence of SEQ ID NO: 4,
(b) a transmembrane domain, and
(c) an intracellular signaling domain.

In some embodiments, the initial therapy further comprises (1) 4-8 cycles of an induction therapy and (2) a high-dose chemotherapy.

In some embodiments, the stem cell transplantation is autologous or allogeneic stem cell transplantation. In some embodiments, the stem cell transplantation is autologous stem cell transplantation (ASCT). In some embodiments, the stem cell transplantation is tandem ASCT.

In some embodiments, the high-dose chemotherapy comprises melphalan.

In some embodiments, the induction therapy comprises a proteasomal inhibitor (PI) or an immunomodulatory drug (IMiD). In some embodiments, the PI is bortezomib, carfilzomib, or ixazomib. In some embodiments, the IMiD is lenalidomide, pomalidomide, thalidomide, or a cereblon E3 ligase modulatory drug (CELMoD), such as iberdomine or mezigdomide. In some embodiments, the induction therapy further comprises an alkylating agent. In some embodiments, the alkylating agent is cyclophosphamide. In some embodiments, the induction therapy further comprises an anti-CD38 antibody.

In some embodiments, the method further comprises administering to the subject a dose of an immunomodulatory drug (IMiD) after administering to the subject the dose of the T cells.

In some embodiments, the subject is not refractory to the IMiD administered after the dose of the T cells. In some embodiments, the IMiD is lenalidomide. In some embodiments, the dose of lenalidomide is about 2.5 mg, about 5 mg, about 10 mg, or about 15 mg daily. In some embodiments, the dose of lenalidomide is about 2.5 mg daily. In some embodiments, the dose of lenalidomide is about 5 mg daily. In some embodiments, the dose of lenalidomide is about 10 mg daily. In some embodiments, the dose of lenalidomide is about 15 mg daily. In some embodiments, the administering of the dose of lenalidomide is once daily. In some embodiments, the administering of the dose of lenalidomide starts between about 21 days to about 214 days after the administering of the dose of the T cells to the subject. In some embodiments, the administering of the dose of lenalidomide starts at a median of about 51 days or at a mean of about 85.1 days. In some embodiments, the administering of the dose of lenalidomide continues for between about 70 days to about 716 days. In some embodiments, the administering of the dose of lenalidomide continues for a median of about 426.5 days or at a mean of about 426.0 days. In some embodiments, the administering of the dose of lenalidomide is daily in a cycle of about 28 days. In some embodiments, the administering of the dose of lenalidomide continues for between about 3 cycles to about 26 cycles. In some embodiments, the administering of the dose of the lenalidomide continues for a median or a mean of about 15 cycles. In some embodiments, the administering of the dose of the lenalidomide is at a relative dose intensity of between about 67.9% to about 100.0%. In some embodiments, the administering of the dose of the lenalidomide is at a median relative dose intensity of about 93.4%. In some embodiments, the administering of the dose of lenalidomide continues until the later of when the subject is confirmed progressive disease or unacceptable toxicity, or when it has reached 2 years after start of the administering of the dose of lenalidomide.

In some embodiments, the subject has further received a lymphodepletion therapy at least about 5 to about 7 days prior to the administering of the dose of the T cells. In some embodiments, the lymphodepletion therapy comprises administering cyclophosphamide and fludarabine daily. In some embodiments, the lymphodepletion therapy comprises cyclophosphamide at a concentration of about 300 mg/m² and fludarabine at a concentration of about 30 mg/m² daily for 3 days.

In some embodiments, the subject has further received a bridging therapy prior to the lymphodepletion therapy but after the initial therapy. In some embodiments, the bridging therapy comprises lenalidomide. In some embodiments, the bridging therapy comprises at least one cycle of lenalidomide at a dose of about 10 mg per day.

In some embodiments, the dose of the T cells is about 0.5-1.0×10⁶ of the T cells/kg of body weight of the subject. In some embodiments, the dose of the T cells is about 0.56-0.84×10⁶ of the T cells/kg of body weight of the subject. In some embodiments, the dose of the T cells is about 0.68×10⁶ of the T cells/kg of body weight of the subject. In some embodiments, the dose of the T cells is formulated to be about 0.60-0.80×10⁶ of the T cells/kg of body weight of the subject. In some embodiments, the dose of the T cells is formulated to be about 0.70×10⁶ of the T cells/kg of body weight of the subject.

In some embodiments, the administering of the dose of the T cells is in a single, two, or three infusions.

In some embodiments, the method is effective in obtaining minimal residual disease (MRD) negativity assessed in the bone marrow of the subject after the administering to the subject the dose of the T cells. In some embodiments, the MRD negativity is assessed using next generation sequencing (NGS) or next generation flow (NGF) on bone marrow aspirate DNA of the subject, wherein optionally the MRD negativity is assessed at a sensitivity of 10⁻⁵. In some embodiments, the MRD negativity is obtained at about 0.9 month to about 6.1 months after the administering to the subject the dose of the T cells. In some embodiments, the MRD negativity is obtained at a median time of about 1.33 months after the administering to the subject the dose of the T cells. In some embodiments, the MRD negativity is obtained at a mean time of about 2.10 months after the administering to the subject the dose of the T cells. In some embodiments, the method is effective in obtaining the MRD negativity at a rate of between about 44.0% to about 89.7% at a sensitivity of 10⁻⁵. In some embodiments, the method is effective in obtaining the MRD negativity at a rate of about 70.6% at a sensitivity of 10⁻⁵. In some embodiments, the method is effective in obtaining the MRD negativity at a rate of between about 51.9% to about 95.7% at a sensitivity of 10⁻⁵. In some embodiments, the method is effective in obtaining the MRD negativity at a rate of about 80.0% at a sensitivity of 10⁻⁵.

In some embodiments, the method is effective in obtaining at least one response in the subject after the administering to the subject the dose of the T cells, and wherein the at least one response comprises, in order from better to worse:
(i) a stringent complete response;
(ii) a complete response;
(iii) a very good partial response;
(iv) a partial response; or
(v) a minimal response.

In some embodiments, the method is effective in obtaining a first response of any one of a partial response, a very good partial response, a complete response, or a stringent complete response. In some embodiments, the method is effective in obtaining the first response at a time of between about 0.9 month and about 12.5 months after the administering of the dose of the T cells. In some embodiments, the method is effective in obtaining the first response at a mean time of about 3.07 months after the administering of the dose of the T cells. In some embodiments, the method is effective in obtaining a first response at a median time of about 1.30 months after the administering of the dose of the T cells.

In some embodiments, the method is effective in obtaining a best response of any one of a partial response, a very good partial response, a complete response, or a stringent complete response. In some embodiments, the method is effective in obtaining the best response at a time of between about 0.9 month and about 12.5 months after the administering of the dose of the T cells. In some embodiments, the method is effective in obtaining the best response at a mean time of about 4.02 months after the administering of the dose of the T cells. In some embodiments, the method is effective in obtaining the best response at a median time of about 1.89 months after the administering of the dose of the T cells.

In some embodiments, the method is effective in obtaining a complete response or a stringent complete response at a time of between about 0.9 month and about 12.5 months after the administering of the dose of the T cells. In some embodiments, the method is effective in obtaining the complete response or the stringent complete response at a mean time of about 3.30 months after the administering of the dose of the T cells. In some embodiments, the method is effective in obtaining the complete response or the stringent complete response at a median time of about 1.72 months after the administering of the dose of the T cells. In some embodiments, the method is effective in obtaining a complete response or a stringent complete response at a rate of between about 71.3% and about 99.9%. In some embodiments, the method is effective in obtaining the complete response or the stringent complete response at a rate of about 94.1%.

In some embodiments, the method is effective in obtaining a very good partial response, a complete response or a stringent complete response at a rate of between about 71.3% and about 99.9%. In some embodiments, the method is effective in obtaining the very good partial response, the complete response or the stringent complete response at a rate of about 94.1%.

In some embodiments, the method is effective in obtaining a partial response, a very good partial response, a complete response or a stringent complete response at a rate of between about 71.3% and about 99.9%. In some embodiments, the method is effective in obtaining the partial response, the very good partial response, the complete response or the stringent complete response at a rate of about 94.1%.

In some embodiments, the method is effective in obtaining a minimal response, a partial response, a very good partial response, a complete response or a stringent complete response at a rate of between about 71.3% and about 99.9%. In some embodiments, the method is effective in obtaining the minimal response, the partial response, the very good partial response, the complete response or the stringent complete response at a rate of about 94.1%.

In some embodiments, the method is effective in obtaining a best response comprising a stringent complete response at a rate of between about 63.6% and about 98.5%. In some embodiments, the method is effective in obtaining the stringent complete response at a rate of about 88.2%.

In some embodiments, the method is effective in obtaining a best response comprising a complete response or a stringent complete response and further comprising a MRD negativity at a rate of between about 50.1% and about 93.2%. In some embodiments, the method is effective in obtaining the best response at a rate of about 76.5%.

In some embodiments, the method is effective in obtaining a best response comprising a complete response at a rate of between about 0.1% and about 28.7%, wherein optionally the method is effective in obtaining the best response at a rate of about 5.9%.

In some embodiments, the method is effective in maintaining the response for at least about 6 months at a rate of about 100%. In some embodiments, the method is effective in maintaining the response for at least about 9 months at a rate of about 100%. In some embodiments, the method is effective in maintaining the response for at least about 12 months at a rate of about 100%.

In some embodiments, the method is effective in obtaining progression-free survival of the subject. In some embodiments, the method is effective in obtaining the progression-free survival at a rate of about 100.0% at a follow-up time of about 6 months. In some embodiments, the method is effective in obtaining the progression-free survival at a rate of about 100.0% at a follow-up time of about 9 months. In some embodiments, the method is effective in obtaining the progression-free survival at a rate of between about 63.2% and about 99.1% at a follow-up time of about 12 months. In some embodiments, the method is effective in obtaining the progression-free survival at a rate of about 93.8% at a follow-up time of about 12 months. In some embodiments, the method is effective in obtaining the progression-free survival at a rate of between about 63.2% and about 99.1% at a follow-up time of about 18 months. In some embodiments, the method is effective in obtaining the progression-free survival at a rate of about 93.8% at a follow-up time of about 18 months.

In some embodiments, the method is effective in obtaining an overall survival rate. In some embodiments, the overall survival rate is about 100.0% at a follow-up time of about 6 months. In some embodiments, the overall survival rate is about 100.0% at a follow-up time of about 9 months. In some embodiments, the overall survival rate is between about 63.2% and about 99.1% at a follow-up time of about 12 months. In some embodiments, the overall survival rate is about 93.8% at a follow-up time of about 12 months. In some embodiments, the overall survival rate is between about 63.2% and about 99.1% at a follow-up time of about 18 months. In some embodiments, the overall survival rate is about 93.8% at a follow-up time of about 18 months.

In some embodiments, the method further comprises treating the subject for an adverse event after the administering of the dose of the T cells. In some embodiments, the method comprises administering a treatment to the subject to alleviate the adverse event.

In some embodiments, the adverse event comprises a treatment-emergent adverse event. In some embodiments, the treatment-emergent adverse event is a serious treatment-emergent adverse event.

In some embodiments, the treatment-emergent adverse event occurs within the later of about 100 days at or after the administering of the dose of the T cells or about 30 days after last dose of lenalidomide.

In some embodiments, severity of the treatment-emergent adverse event is Grade 1, Grade 2, Grade 3, or Grade 4. In some embodiments, the severity of the treatment-emergent adverse event is Grade 3 or 4, wherein optionally maximum severity of treatment-emergent adverse event is Grade 4. In some embodiments, maximum severity of the treatment-emergent adverse event is Grade 3. In some embodiments, the maximum severity of the treatment-emergent adverse event of Grade 3 occurs at a rate of about 17.6%. In some embodiments, maximum severity of the treatment-emergent adverse event is Grade 4. In some embodiments, the maximum severity of the treatment-emergent adverse event of Grade 4 occurs at a rate of about 82.4%.

In some embodiments, the treatment-emergent adverse event comprises a blood or lymphatic system disorder, an immune system disorder, a gastrointestinal disorder, an infection, an infestation, a musculoskeletal or connective tissue disorder, a respiratory, thoracic or mediastinal disorder, a general disorder, an administrate site condition, a metabolism or nutrition disorder, a nervous system disorder, a skin or subcutaneous tissue disorder, an eye disorder, a cardiac disorder, an ear or labyrinth disorder, or a vascular disorder. In some embodiments, the treatment-emergent adverse event occurs at a rate of at least about 10%.

In some embodiments, the treatment-emergent adverse event comprises neutropenia, lymphopenia, thrombocytopenia, leukopenia, anemia, febrile neutropenia, cytokine release syndrome, hypogammaglobulinaemia, diarrhoea, nausea, abdominal distension, abdominal pain, upper abdominal pain, constipation, dyspepsia, vomiting, increased aspartate aminotransferase, increased alanine aminotransferase, increased blood lactate dehydrogenase, decreased CD4 lymphocytes, increased gamma-glutamyltransferase, decreased serum ferritin, an upper respiratory tract infection, COVID-19, nasopharyngitis, pneumonia, respiratory syncytial virus infection, Rhinovirus infection, sinusitis, myalgia, back pain, muscle spasm, musculoskeletal stiffness, pain in extremity, cough, productive cough, nasal congestion, rhinorrhoea, epistaxis, oropharyngeal pain, fatigue, chill, malaise, pyrexia, hyperferritinaemia, hypoalbuminaemia, hypocalcaemia, hypomagnesaemia, hypophosphataemia, headache, dysarthria, hyperhidrosis, pruritus, or sinus tachycardia. In some embodiments, the treatment-emergent adverse event occurs at a rate of at least about 10%.

In some embodiments, the treatment-emergent adverse event is a serious treatment-emergent adverse event that occurs at a rate of about 58.8%. In some embodiments, maximum severity of the treatment-emergent adverse event is Grade 3 or Grade 4. In some other embodiments, the maximum severity of the treatment-emergent adverse event of Grade 3 or Grade 4 occurs at a rate of about 52.9%.

In some embodiments, the serious treatment-emergent adverse event comprises an infection, an infestation, a nervous system disorder, a blood or lymphatic system disorder, an immune system disorder, an eye disorder, a gastrointestinal disorder, a musculoskeletal or connective tissue disorder, or a vascular disorder. In some embodiments, the serious treatment-emergent adverse event occurs at a rate of at least about 2%.

In some embodiments, the serious treatment-emergent adverse event comprises pneumonia, Rhinovirus infection, COVID-19 pneumonia, metapneumovirus infection, influenzal pneumonia, viral pneumonia, respiratory syncytial virus infection, sinusitis, streptococcal sepsis, facial nerve disorder, facial paralysis, peripheral motor neuropathy, febrile neutropenia, neutropenia, cytokine release syndrome, diplopia, diarrhoea, musculoskeletal pain, or haematoma. In some embodiments, the serious treatment-emergent adverse event occurs at a rate of at least about 2%.

In some embodiments, the treatment-emergent adverse event comprises cytokine release syndrome (CRS), wherein optionally the CRS occurs at a rate of about 82.4%, wherein optionally:
(1) time to first onset of the CRS ranges from about 6 days to about 11 days after the administering of the dose of the T cells to the subject, wherein further optionally the time to the first onset of the CRS is at a median of about 8.0 days or at a mean of about 8.1 days;
(2) time to recovery of the CRS ranges from about 1 day to about 5 days, wherein further optionally the time to recovery of the CRS is at a median time of about 2.5 days or at a mean time of about 2.6 days;
(3) duration of the CRS ranges from about 1 day to about 5 days or from about 2 days to about 3 days, wherein further optionally the duration of the CRS is at a median time of about 2.5 days or at a mean time of about 2.6 days; or
(4) duration of the CRS of less than about 7 days occurs at a rate of about 100%.

In some embodiments, the CRS occurs at maximum toxicity grade of Grade 1, Grade 2, Grade 3, Grade 4, or Grade 5, wherein optionally:
(1) the CRS occurs at the maximum toxicity grade of Grade 1 at a rate of about 76.5%; or
(2) the CRS occurs at the maximum toxicity grade of Grade 2 at a rate of about 5.9%.

In some embodiments, the treatment comprises an anti-IL-6 receptor, an IL-1 receptor antagonist, a corticosteroid, IV fluids, a vasopressor, oxygen, an analgesic, an anti-inflammatory drug, an antiinfective, an antiepileptic, caffeine, or prochlorperazine, or any combination thereof, wherein optionally:
(1) the anti-IL-6 receptor comprises tocilizumab;
(2) the IL-1 receptor antagonist comprises anakinra; or
(3) oxygen comprises blow-by, nasal cannula low flow at a flow rate of at most about 6 L/min, nasal cannula high flow at a flow rate of at least about 6 L/min, face mask, non-rebreather mask, venturi mask, or positive pressure, or any combination thereof.

In some embodiments, the treatment is effective at achieving a recovery or resolution of the CRS, wherein optionally the CRS is recovered or resolved at a rate of about 100%.

In some embodiments, the treatment-emergent adverse event comprises immune effector cell-associated neurotoxicity (ICANS), wherein optionally the ICANS occurs at a rate of about 5.9%, and wherein optionally:
(1) time to first onset of the ICANS is about 7 days after the administering of the dose of the T cells to the subject, wherein further optionally the time to the first onset of the ICANS is at a median of about 7 days or at a mean of about 7 days;
(2) time to recovery of the ICANS is about 1 day, wherein further optionally the time to recovery of the ICANS is at a median time of about 1 day or at a mean time of about 1 day;
(3) duration of the ICANS is about 1 day, wherein further optionally the duration of the ICANS is at a median time of about 1 day or at a mean time of about 1 day; or
(4) the ICANS occurs concurrently with cytokine release syndrome (CRS) at a rate of about 5.9%.

In some embodiments, the ICANS occurs at maximum toxicity grade of Grade 1, Grade 2, Grade 3, or Grade 4. In some embodiments, the ICANS occurs at maximum toxicity grade of Grade 1 at a rate of about 5.9%.

In some embodiments, the treatment comprises an anti-IL-6 receptor, an IL-1 receptor antagonist, a corticosteroid, or ceftazidime, or any combination thereof, wherein optionally:
(1) the anti-IL-6 receptor comprises tocilizumab; or
(2) the IL-1 receptor antagonist comprises anakinra.

In some embodiments, the ICANS is recovered, resolved, not recovered, not resolved, recovered with sequelae, resolved with sequelae, recovering, resolving, or unknown, wherein optionally the treatment is effective at achieving a recovery or resolution of the ICANS at a rate of about 100%.

In some embodiments, the adverse event comprises neurotoxicity, wherein optionally the neurotoxicity occurs at a rate of about 35.3%, wherein optionally:
(1) time to first onset of the neurotoxicity ranges from about 16 days to about 27 days after the administering of the dose of the T cells to the subject, wherein further optionally the time to the first onset of the neurotoxicity is at a median of about 21.0 days or at a mean of about 20.8 days;
(2) time to recovery of the neurotoxicity ranges from about 29 days to about 443 days, wherein further optionally the time to recovery of the neurotoxicity is at a median time of about 70.0 days or at a mean time of about 153.0 days;
(3) duration of the neurotoxicity ranges from about 29 days to about 791 days, wherein further optionally the duration of the neurotoxicity is at a median time of about 111.0 days or at a mean time of about 254.7 days.

In some embodiments, the neurotoxicity is recovered, resolved, not recovered, or not resolved, wherein optionally the neurotoxicity is recovered or resolved at a rate of about 23.5%, and wherein optionally the neurotoxicity is not recovered or not resolved at a rate of about 11.8%.

In some embodiments, the adverse event is an adverse event of special interest, wherein optionally:
(1) the adverse event of special interest occurs at a rate of about 82.4%, wherein further optionally the adverse event of special interest occurs at least at Grade 3 at a rate of about 23.5%; or
(2) the adverse event of special interest comprises cytokine release syndrome (CRS), CAR-T cell related neurotoxicity, second primary malignancy, or a movement and neurocognitive treatment-emergent adverse event, or any combination thereof.

In some embodiments, the adverse event of special interest is CRS, wherein optionally the CRS occurs at a rate of about 82.4%, wherein further optionally the CRS occurs at least at Grade 3 at a rate of about 0%.

In some embodiments, the adverse event of special interest is CAR-T cell related neurotoxicity, wherein optionally:
(1) the CAR-T cell related neurotoxicity occurs at a rate of about 35.3%, wherein further optionally the CAR-T cell related neurotoxicity occurs at least at Grade 3 at a rate of about 5.9%; or
(2) the CAR-T cell related neurotoxicity comprises immune effector cell-associated neurotoxicity (ICANS) or other neurotoxicity, wherein further optionally the ICANS occurs at a rate of about 5.9%, wherein further optionally the ICANS occurs at least at Grade 3 at a rate of about 0%, wherein further optionally the other neurotoxicity occurs at a rate of about 35.3%, and wherein further optionally the other neurotoxicity occurs at least at Grade 3 at a rate of about 5.9%.

In some embodiments, the adverse event of special interest is second primary malignancy, wherein optionally the second primary malignancy comprises myelodysplastic syndrome, wherein optionally the second primary malignancy occurs at a rate of about 5.9%, wherein further optionally the second primary malignancy occurs at least at Grade 3 at a rate of about 5.9%.

In some embodiments, the adverse event of special interest is a movement and neurocognitive treatment-emergent adverse event, wherein optionally the movement and neurocognitive treatment-emergent adverse event does not occur in the subject.

In some embodiments, the adverse event comprises prolonged cytopenia, wherein optionally the prolonged cytopenia comprises thrombocytopenia, neutropenia, lymphopenia, or anemia, or any combination thereof, wherein further optionally:
(1) thrombocytopenia occurs at Grade 3 or 4 at a rate of about 29.4% after the administering of the dose of the T cells to the subject, wherein further optionally thrombocytopenia recovers to Grade 2 or less by about 30 days or about 60 days, and wherein further optionally thrombocytopenia reoccurs at Grade 3 or 4 at a rate of about 0%;
(2) neutropenia occurs at Grade 3 or 4 at a rate of about 88.2% after the administering of the dose of the T cells to the subject, wherein further optionally neutropenia recovers to Grade 2 or less by about 30 days or about 60 days, and wherein further optionally neutropenia reoccurs at Grade 3 or 4 at a rate of about 35.3%;
(3) lymphopenia occurs at Grade 3 or 4 at a rate of about 100% after the administering of the dose of the T cells to the subject, wherein further optionally lymphopenia recovers to Grade 2 or less by about 30 days or about 60 days, and wherein further optionally lymphopenia reoccurs at Grade 3 or 4 at a rate of about 11.8%; or
(4) anemia occurs at Grade 3 or 4 at a rate of about 5.9% after the administering of the dose of the T cells to the subject, wherein further optionally anemia recovers to Grade 2 or less by about 30 days or about 60 days, and wherein further optionally anemia reoccurs at Grade 3 or 4 at a rate of about 0%.

In some embodiments, the treatment-emergent adverse event comprises a treatment-emergent infection, wherein optionally:
(1) the treatment-emergent infection occurs at a rate of about 70.6%, wherein further optionally the treatment-emergent infection occurs at Grade 3 or 4 at a rate of about 29.4%;
(2) the treatment-emergent infection comprises an infection, an infestation, a viral infectious disorder, a bacterial infectious disorder, or a fungal infectious disorder, or any combination thereof,
(3) the treatment-emergent infection comprises an upper respiratory tract infection, nasopharyngitis, pneumonia, sinusitis, acute sinusitis, bronchitis, infectious enterocolitis, gastroenteritis, pharyngitis, COVID-19, respiratory syncytial virus infection, Rhinovirus infection, COVID-19 pneumonia, herpes zoster, influenza, metapneumovirus infection, oral herpes, influenzal pneumonia, viral pneumonia, Campylobacter infection, Enterococcal infection, bacterial respiratory tract infection, Streptococcal sepsis, Aspergillus infection, Candida infection, fungal foot infection, or tongue fungal infection, or any combination thereof.

In some embodiments, the first VHH domain comprises a CDR1 comprising the amino acid sequence of SEQ ID NO: 18, a CDR2 comprising the amino acid sequence of SEQ ID NO: 19, a CDR3 comprising the amino acid sequence of SEQ ID NO: 20, and the second VHH domain comprises a CDR1 comprising the amino acid sequence of SEQ ID NO: 21, a CDR2 comprising the amino acid sequence of SEQ ID NO: 22, and a CDR3 comprising the amino acid sequence of SEQ ID NO: 23, wherein optionally the first VHH domain comprises the amino acid sequence of SEQ ID NO: 2 and the second VHH domain comprises the amino acid sequence of SEQ ID NO: 4, wherein optionally the first VHH domain is at the N-terminus of the second VHH domain, or the first VHH domain is at the C-terminus of the second VHH domain, further wherein optionally:
(a) the first VHH domain is linked to the second VHH domain via a linker, wherein optionally the linker comprises the amino acid sequence of SEQ ID NO: 3;
(b) the transmembrane domain is derived from a molecule selected from the group consisting of CD8a, CD4, CD28, CD137, CD80, CD86, CD152 and PD1, wherein optionally the transmembrane domain is derived from CD8a, wherein optionally the transmembrane domain comprises the amino acid sequence of SEQ ID NO: 6;
(c) the intracellular signaling domain comprises a primary intracellular signaling domain of an immune effector cell, wherein optionally the primary intracellular signaling domain is derived from CD3ζ, wherein optionally the primary intracellular signaling domain comprises the amino acid sequence of SEQ ID NO: 8;
(d) the intracellular signaling domain comprises a co-stimulatory signaling domain, wherein optionally the co-stimulatory signaling domain is derived from a co-stimulatory molecule selected from the group consisting of CD27, CD28, CD137, OX40, CD30, CD40, CD3, LFA-1, ICOS, CD2, CD7, LIGHT, NKG2C, B7-H3, ligands of CD83 and any combination thereof, wherein optionally the co-stimulatory signaling domain comprises a cytoplasmic domain of CD137, wherein optionally the co-stimulatory signaling domain comprises the amino acid sequence of SEQ ID NO: 7;
(e) the CAR further comprises a hinge domain located between the C-terminus of the extracellular antigen binding domain and the N-terminus of the transmembrane domain, wherein optionally the hinge domain is derived from CD8a, wherein optionally the hinge domain comprises the amino acid sequence of SEQ ID NO: 5;
(f) the CAR further comprises a signal peptide located at the N-terminus of the polypeptide, wherein optionally the signal peptide is derived from CD8a comprising the amino acid sequence of SEQ ID NO: 1; or
(g) the CAR comprises the amino acid sequence of SEQ ID NO: 17.

In some embodiments, the dose of the T cells is formulated in a composition comprising dimethyl sulfoxide (DMSO), wherein optionally the DMSO is at a concentration of about 5%.

In one aspect is provided a method of treating a subject with multiple myeloma, wherein the subject has not achieved a complete response after receiving an initial therapy comprising (1) 4 to 8 cycles of an induction therapy, (2) a high-dose chemotherapy, and (3) an autologous stem cell transplantation (ASCT), the method comprising:
(1) administering to the subject a dose of T cells comprising a chimeric antigen receptor (CAR) comprising:
   (a) an extracellular antigen binding domain capable of specifically binding to an epitope of B-cell maturation antigen (BCMA), wherein the extracellular antigen binding domain comprises a first VHH domain comprising the amino acid sequence of SEQ ID NO: 2 and a second VHH domain comprising the amino acid sequence of SEQ ID NO: 4,
   (b) a transmembrane domain comprising the amino acid sequence of SEQ ID NO: 6 and
   (c) an intracellular signaling domain comprising the amino acid sequence of SEQ ID NO: 8, and
(2) optionally administering to the subject a dose of an immunomodulatory drug (IMiD) after administering to the subject the dose of the T cells.

In one aspect is provided a method of treating a subject with multiple myeloma, wherein the subject has not achieved a complete response after receiving an initial therapy comprising (1) 4 to 8 cycles of an induction therapy, (2) a high-dose chemotherapy, and (3) an autologous stem cell transplantation (ASCT), the method comprising:
(1) administering to the subject a dose of T cells comprising a chimeric antigen receptor (CAR) comprising the amino acid sequence of SEQ ID NO: 17, and
(2) optionally administering to the subject a dose of an immunomodulatory drug (IMiD) after administering to the subject the dose of the T cells.

### 3. BRIEF DESCRIPTION OF THE DRAWINGS

FIG. 1 shows the expression of BCMA antigen on the surface of GC, memory and plasmablast cells in the lymph node, long-lived plasma cells in the bone marrow LN and MALT, and on multiple myeloma cells. BAFF-R antigen is not expressed on plasmablast cells, long-lived plasma cells, or multiple myeloma cells. TACI is expressed on memory and plasmablast cells, long-lived plasma cells, and multiple myeloma cells. CD138 is expressed only on long-lived plasma cells and multiple myeloma cells.

FIG. 2 shows the design of the ciltacabtagene autoleucel CAR. Ciltacabtagene autoleucel comprises two VHH domains, as opposed to a single VL domain and a single VH domain found on various other CARs. Ciltacabtagene autoleucel comprises intracellular CD137 and human CD3 zeta domains.

FIG. 3 shows a schematic for preparing virus encoding ciltacabtagene autoleucel CAR, transduction of the virus into a T cell from the patient, and then preparation of CAR T cells expressing ciltacabtagene autoleucel.

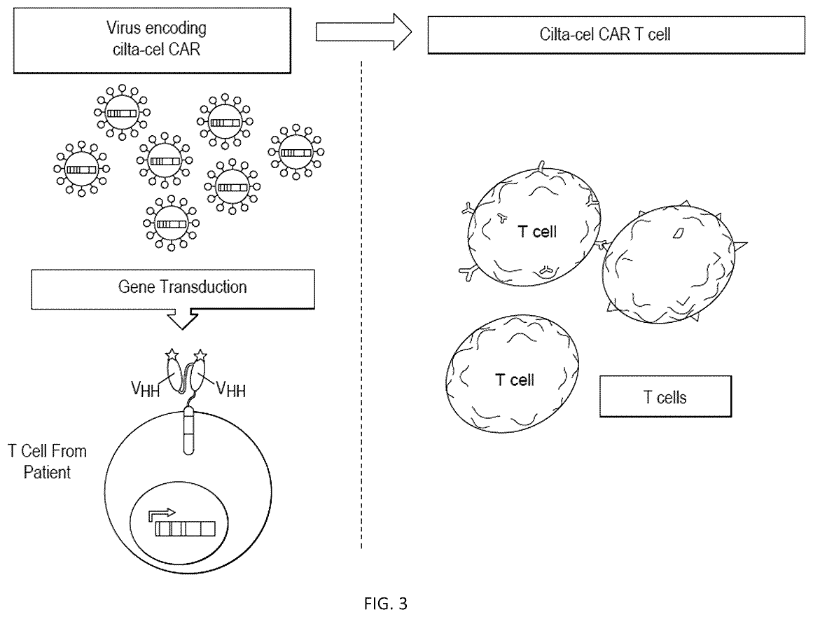

FIG. 4 shows a schematic of study design for ciltacabtagene autoleucel CAR T-cells. The patient population includes those with Multiple Myeloma, without complete response (best response <complete response and ≥stable disease) after receiving an initial therapy comprising (1) 4 to 8 total cycles of an induction therapy, (2) a high-dose chemotherapy and (3) an ASCT with or without consolidation. A primary objective is efficacy: minimal residual disease (MRD) negative rate as defined by the International Myeloma Working Group (IMWG) criteria (Phase 2). A secondary efficacy objective is efficacy: ORR—PR or better as defined by IMWG (Phase 2). The following are other secondary objectives: Incidence and severity of adverse events (Phase 2), and any further efficacy characterization.

FIG. 5 shows a schematic of an exemplary protocol design for ciltacabtagene autoleucel CAR T-cells. The patient population includes those with Multiple Myeloma, without complete response (best response <complete response and ≥stable disease) after the initial therapy comprising (1) 4 to 8 total cycles of an induction therapy, (2) a high-dose chemotherapy and (3) an ASCT with or without consolidation. Bridging therapy was allowed when clinically indicated, with the permission of the sponsor. Additional cycles or alternative therapy was permitted with sponsor approval. The lenalidomide was administered when applicable after the ciltacabtagene autoleucel CAR T-cells infusion, following a staggered dosing strategy.

FIG. 6 shows the major secondary efficacy endpoints, e.g., overall response, assessed based on computerized algorithm as defined by the IMWG.

FIG. 7 shows the major secondary efficacy endpoints, e.g., duration of response (DOR), assessed based on computerized algorithm as defined by the IMWG. The Kaplan-Meier plot for DOR was based on computerized algorithm assessment of responders from Cohort D in all treated analysis Set (Study 68284528MMY2003).

### 4. DETAILED DESCRIPTION

The disclosure provides methods of treating patients with multiple myeloma with CAR-T cells. In certain embodiments, the methods relate to treating patients with multiple myeloma who has not achieved a complete response after receiving an initial therapy comprising a stem cell transplantation. The disclosure also provides related nucleic acids, recombinant expression vectors, host cells, populations of cells, antibodies, or antigen binding portions thereof, and pharmaceutical compositions relating to the immune cells and CAR-expressing T cells of the disclosure. Dosage regimens and dosage forms are also provided.

Several aspects of the disclosure are described below, with reference to examples for illustrative purposes only. It should be understood that numerous specific details, relationships, and methods are set forth to provide a full understanding of the disclosure. One having ordinary skill in the relevant art, however, will readily recognize that the disclosure can be practiced without one or more of the specific details or practiced with other methods, protocols, reagents, cell lines and animals. The present disclosure is not limited by the illustrated ordering of acts or events, as some acts may occur in different orders and/or concurrently with other acts or events. Furthermore, not all illustrated acts, steps or events are required to implement a methodology in accordance with the present disclosure.

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly used dictionaries, should be interpreted as having a meaning that is consistent with their meaning in the context of the relevant art and/or as otherwise defined herein.

### 4.1. Definitions

The term “about” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

The term “protein” or “polypeptide” is used herein encompasses all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation, ADP-ribosylation, pegylation, biotinylation, etc.).

The terms “nucleic acid”, “nucleotide”, and “polynucleotide” encompass both DNA and RNA unless specified otherwise. By a “nucleic acid sequence” or “nucleotide sequence” is meant the nucleic acid sequence encoding an amino acid; these terms may also refer to the nucleic acid sequence including the portion coding for any amino acids added as an artifact of cloning, including any amino acids coded for by linkers.

The term “antibody” includes monoclonal antibodies (including full length 4-chain antibodies or full length heavy-chain only antibodies which have an immunoglobulin Fc region), antibody compositions with polyepitopic specificity, multispecific antibodies (e.g., bispecific antibodies, diabodies, and single-chain molecules), as well as antibody fragments (e.g., Fab, F(ab′)2, and Fv). The term “immunoglobulin” (Ig) is used interchangeably with “antibody” herein. Antibodies contemplated herein include single-domain antibodies, such as heavy chain only antibodies. The terms “antibody” and “antibodies” refer to monoclonal antibodies, multispecific antibodies, human antibodies, humanized antibodies, chimeric antibodies, single-chain Fvs (scFv), single chain antibodies, Fab fragments, F(ab′) fragments, disulfide-linked Fvs (sdFv), intrabodies, minibodies, diabodies and anti-idiotype (anti-Id) antibodies (including, e.g., anti-Id antibodies to antigen specific TCR), and epitope-binding fragments of any of the above. The terms “antibody” and “antibodies” also refer to covalent diabodies such as those disclosed in U.S. Pat. Appl. Pub. 2007/0004909 and Ig-DARTS such as those disclosed in U.S. Pat. Appl. Pub. 2009/0060910. Immunoglobulin molecules can be of any type (e.g., IgG, IgE, IgM, IgD, IgA and IgY), class (e.g., IgG1, IgG2, IgG3, IgG4, IgM1, IgM2, IgA1 and IgA2) or subclass.

A “full length antibody” is comprised of two heavy chains (HC) and two light chains (LC) inter-connected by disulfide bonds as well as multimers thereof (e.g. IgM). Each heavy chain is comprised of a heavy chain variable domain (VH) and a heavy chain constant domain, the heavy chain constant domain comprised of subdomains CH1, hinge, CH2 and CH3. Each light chain is comprised of a light chain variable domain (VL) and a light chain constant domain (CL). The VH and the VL may be further subdivided into regions of hypervariability, termed complementarity determining regions (CDR), interspersed with framework regions (FW). Each VH and VL is composed of three CDRs and four FW segments, arranged from amino-to-carboxy-terminus in the following order: FW1, CDR1, FW2, CDR2, FW3, CDR3 and FW4.

“Complementarity determining regions (CDR)” are antigen-binding sites in an antibody. CDRs may be defined using various terms: (i) Complementarity Determining Regions (CDRs), three in the VH (HCDR1, HCDR2, HCDR3) and three in the VL (LCDR1, LCDR2, LCDR3) are based on sequence variability (Wu and Kabat, J. Exp. Med. 132:211-50, 1970; Kabat et al., Sequences of Proteins of Immunological Interest, 5th Ed. Public Health Service, National Institutes of Health, Bethesda, Md., 1991). (ii) “Hypervariable regions”, “HVR”, or “HV”, three in the VH (H1, H2, H3) and three in the VL (L1, L2, L3) refer to the regions of an antibody variable domains which are hypervariable in structure as defined by Chothia and Lesk (Chothia and Lesk, Mol. Biol. 196:901-17, 1987). The International ImMunoGeneTics (IMGT) database (http://www_imgt_org) provides a standardized numbering and definition of antigen-binding sites. The correspondence between CDRs, HVs and IMGT delineations is described in Lefranc et al., Dev. Comparat. Immunol. 27:55-77, 2003. The term “CDR”, “HCDR1”, “HCDR2”, “HCDR3”, “LCDR1”, “LCDR2” and “LCDR3” as used herein includes CDRs defined by any of the methods described supra, Kabat, Chothia or IMGT, unless otherwise explicitly stated in the specification. The framework regions (FW) are adjacent to and between the CDRs in both the VL (LFW1, LFW2, LFW3, LFW4) and VH (HFW1, HFW2, HFW3, HFW4).

The term “heavy chain-only antibody” or “HCAb” refers to a functional antibody, which comprises heavy chains, but lacks the light chains usually found in 4-chain antibodies. Camelid animals (such as camels, llamas, or alpacas) are known to produce HCAbs.

The term “single-domain antibody” or “sdAb” refers to a single antigen-binding polypeptide having three complementary determining regions (CDRs). The sdAb alone is capable of binding to the antigen without pairing with a corresponding CDR-containing polypeptide. In some cases, single-domain antibodies are engineered from camelid HCAbs, and their heavy chain variable domains are referred herein as “VHHs”. Some VHHs may also be known as “Nanobodies”. A camelid sdAb is one of the smallest known antigen-binding antibody fragments (see, e.g., Hamers-Casterman et al., Nature 363:446-8 (1993); Greenberg et al., Nature 374:168-73 (1995); Hassanzadeh-Ghassabeh et al., Nanomedicine (Lond), 8:1013-26 (2013)). A basic VHH has the following structure from the N-terminus to the C-terminus: FR1-CDR1-FR2-CDR2-FR3-CDR3-FR4, in which FR1 to FR4 refer to framework regions 1 to 4, respectively, and in which CDR1 to CDR3 refer to the complementarity determining regions 1 to 3.

The “variable region” or “variable domain” of an antibody refers to the amino-terminal domains of the heavy or light chain of the antibody. The variable domains of the heavy chain and light chain may be referred to as “VH” and “VL”, respectively. These domains are generally the most variable parts of the antibody (relative to other antibodies of the same class) and contain the antigen binding sites. Heavy-chain only antibodies from the Camelid species have a single heavy chain variable region, which is referred to as “VHH”. VHH is thus a special type of VH.

The term “variable” refers to the fact that certain segments of the variable domains differ extensively in sequence among antibodies. The V domain (i.e., variable domain) mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy-chain variable domains. The more highly conserved portions of variable domains are called the framework regions (FR). The variable domains of native heavy and light chains each comprise four FR regions, largely adopting a β-sheet configuration, connected by three HVRs, which form loops connecting, and in some cases forming part of, the j-sheet structure. The HVRs in each chain are held together in close proximity by the FR regions and contribute to the formation of the antigen binding site of antibodies (with the HVRs from the other chain, if the antibody is not a sdAb) (see Kabat et al., Sequences of Immunological Interest, Fifth Edition, National Institute of Health, Bethesda, Md. (1991)). The constant domains are not involved directly in the binding of antibody to an antigen, but exhibit various effector functions, such as participation of the antibody in antibody-dependent cellular toxicity.

The terms “fragment of an antibody”, “antibody fragment”, “functional fragment of an antibody”, and “antigen-binding portion” are used interchangeably herein to mean one or more fragments or portions of an antibody that retain the ability to specifically bind to an antigen (see, generally, Holliger et al., Nat. Biotech., 23(9): 1 126-1129 (2005)). The antigen recognition moiety of the CAR encoded by the nucleic acid sequence disclosed herein can contain any BCMA-binding antibody fragment. The antibody fragment desirably comprises, for example, one or more CDRs, the variable region (or portions thereof), the constant region (or portions thereof), or combinations thereof. Examples of antibody fragments include, but are not limited to, (i) a Fab fragment, which is a monovalent fragment consisting of the VL, VH, CL, and CHI domains; (ii) a F(ab′)2 fragment, which is a bivalent fragment comprising two Fab fragments linked by a disulfide bridge at the hinge region; (iii) a Fv fragment consisting of the VL and VH domains of a single arm of an antibody; (iv) a single chain Fv (scFv), which is a monovalent molecule consisting of the two domains of the Fv fragment (i.e., VL and VH) joined by a synthetic linker which enables the two domains to be synthesized as a single polypeptide chain (see, e.g., Bird et al., Science, 242: 423-426 (1988); Huston et al., Proc. Natl. Acad. Sci. USA, 85: 5879-5883 (1988); and Osbourn et al., Nat. Biotechnol, 16: 778 (1998)) and (v) a diabody, which is a dimer of polypeptide chains, wherein each polypeptide chain comprises a VH connected to a VL by a peptide linker that is too short to allow pairing between the VH and VL on the same polypeptide chain, thereby driving the pairing between the complementary domains on different VH-VL polypeptide chains to generate a dimeric molecule having two functional antigen binding sites. Antibody fragments are known in the art and are described in more detail in, e.g., U.S. Patent Application Publication 2009/0093024 A1. Antigen binding fragments may be synthetic, enzymatically obtainable or genetically engineered polypeptides and include portions of an immunoglobulin that bind an antigen, such as the VH, the VL, the VH and the VL, Fab, Fab′, F(ab′)₂, Fd and Fv fragments, domain antibodies (dAb) consisting of one VH domain or one VL domain, shark variable IgNAR domains, camelized VH domains, VHH domains, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3, alternative scaffolds that bind an antigen, and multispecific proteins comprising the antigen binding fragments. Antigen binding fragments (such as VH and VL) may be linked together via a synthetic linker to form various types of single antibody designs where the VH/VL domains may pair intramolecularly, or intermolecularly in those cases when the VH and VL domains are expressed by separate single chains, to form a monovalent antigen binding domain, such as single chain Fv (scFv) or diabody. Antigen binding fragments may also be conjugated to other antibodies, proteins, antigen binding fragments or alternative scaffolds which may be monospecific or multispecific to engineer bispecific and multispecific proteins.

As used herein, the terms “specifically binds”, “specifically recognizes”, or “specific for” refer to measurable and reproducible interactions such as binding between a target and an antigen binding protein (such as a CAR or a VHH), which is determinative of the presence of the target in the presence of a heterogeneous population of molecules including biological molecules.

The term “specificity” refers to selective recognition of an antigen binding protein (such as a CAR or a VHH) for a particular epitope of an antigen. Natural antibodies, for example, are monospecific. The term “multispecific” denotes that an antigen binding protein (such as a CAR or antibody) has two or more antigen-binding sites of which at least two bind different antigen-binding specificities. “Bispecific” as used herein denotes that an antigen binding protein (such as a CAR or antibody) has two different antigen-binding specificities.

As used herein, the term “operatively linked,” and similar phrases, when used in reference to nucleic acids or amino acids, refer to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA). In some embodiments, operatively linked nucleic acid elements result in the transcription of an open reading frame and ultimately the production of a polypeptide (i.e., expression of the open reading frame). As another example, an operatively linked peptide is one in which the functional domains are placed with appropriate distance from each other to impart the intended function of each domain.

A “chimeric antigen receptor” or “CAR” is an artificially constructed hybrid protein or polypeptide containing the antigen binding domains of at least one antibody (or antibody fragment) linked to T-cell signaling domains. Characteristics of CARs can include their ability to redirect T-cell specificity and reactivity toward a selected target in a non-MHC-restricted manner, exploiting the antigen-binding properties of monoclonal antibodies. The non-MHC-restricted antigen recognition gives T cells expressing CARs the ability to recognize antigens independent of antigen processing, thus bypassing a major mechanism of tumor evasion. Moreover, when expressed in T-cells, advantageously, CARs do not dimerize with endogenous T cell receptor (TCR) α- and β-chains. T cells expressing a CAR are referred to herein as CAR T cells, CAR-T cells or CAR modified T cells, and these terms are used interchangeably herein. The cell can be genetically modified to stably express at least one antigen-binding domain on its surface, conferring novel antigen specificity that is MHC independent. “BCMA CAR” refers to a CAR having an extracellular binding domain specific for BCMA. “Bi-epitope CAR” refers to a CAR having an extracellular binding domain specific for two different epitopes of an antigen, such as BCMA.

The terms “B-cell maturation antigen” and “BCMA” as used herein include human B cell maturation antigen, also known as BCMA, CD269, and TNFRSF17 (UniProt Q02223), which is a member of the tumor necrosis receptor superfamily that is preferentially expressed in differentiated plasma cells. The extracellular domain of human BCMA consists, according to UniProt of amino acids 1-54 (or 5-51).

The terms “T cell” and “T lymphocyte” are interchangeable and used synonymously herein. As used herein, T cell includes thymocytes, naive T lymphocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4+ T cell) CD4+ T cell, a cytotoxic T cell (CTL; CD8+ T cell), a tumor infiltrating cytotoxic T cell (TIL; CD8+ T cell), CD4+CD8+ T cell, or any other subset of T cells. Other illustrative populations of T cells suitable for use in particular embodiments include naive T cells and memory T cells. Also included are “NKT cells”, which refer to a specialized population of T cells that express a semi-invariant αβ T-cell receptor, but also express a variety of molecular markers that are typically associated with NK cells, such as NK1.1. NKT cells include NK1.1+ and NK1.1−, as well as CD4+, CD4−, CD8+ and CD8− cells. The TCR on NKT cells is unique in that it recognizes glycolipid antigens presented by the MHC I-like molecule CD1d. NKT cells can have either protective or deleterious effects due to their abilities to produce cytokines that promote either inflammation or immune tolerance. Also included are “gamma-delta T cells (γδ T cells),” which refer to a specialized population that to a small subset of T cells possessing a distinct TCR on their surface, and unlike the majority of T cells in which the TCR is composed of two glycoprotein chains designated α- and β-TCR chains, the TCR in γδ T cells is made up of a γ-chain and a δ-chain. γδ T cells can play a role in immunosurveillance and immunoregulation, and were found to be an important source of IL-17 and to induce robust CD8+ cytotoxic T cell response. Also included are “regulatory T cells” or “Tregs”, which refer to T cells that suppress an abnormal or excessive immune response and play a role in immune tolerance. Tregs are typically transcription factor Foxp3-positive CD4+T cells and can also include transcription factor Foxp3-negative regulatory T cells that are IL-10-producing CD4+T cells.

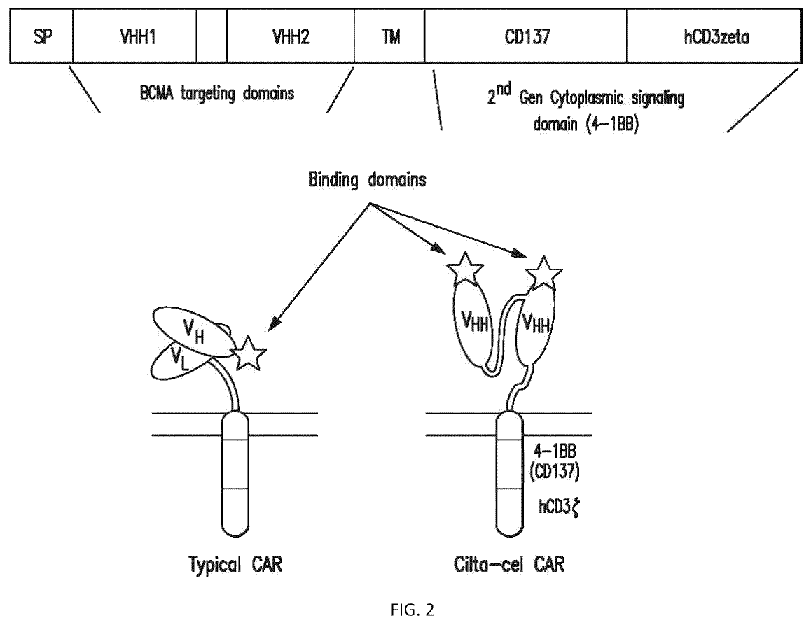

“Ciltacabtagene autoleucel” (“cilta-cel”) is a chimeric antigen receptor T cell (CAR-T) therapy comprising two B-cell maturation antigen (BCMA)-targeting VHH domains designed to confer avidity for BCMA. Cilta-cel can comprise T lymphocytes transduced with the ciltacabtagene autoleucel CAR, a CAR encoded by a lentiviral vector. The CAR targets the human B cell maturation antigen (BCMA CAR). A diagram of the lentiviral vector encoding cilta-cel CAR is provided in FIG. 2. The amino acid sequence of the cilta-cel CAR is the amino acid sequence of SEQ ID NO: 17.

“Tumor cell” or a “cancer cell” refers to a cancerous, pre-cancerous or transformed cell, either in vivo, ex vivo, or in tissue culture, that has spontaneous or induced phenotypic changes. These changes do not necessarily involve the uptake of new genetic material. Although transformation may arise from infection with a transforming virus and incorporation of new genomic nucleic acid, uptake of exogenous nucleic acid or it can also arise spontaneously or following exposure to a carcinogen, thereby mutating an endogenous gene. Transformation/cancer is exemplified by morphological changes, immortalization of cells, aberrant growth control, foci formation, proliferation, malignancy, modulation of tumor specific marker levels, invasiveness, tumor growth in suitable animal hosts such as nude mice, and the like, in vitro, in vivo, and ex vivo.

The terms “express” and “expression” mean allowing for or causing the information in a gene or DNA sequence to become produced. For example, expression can take the form of producing a protein by activating the cellular functions involved in transcription and translation of a corresponding gene or DNA sequence. A DNA sequence is expressed in or by a cell to form an “expression product” such as a protein. The expression product itself, e.g., the resulting protein, may also be said to be “expressed” by the cell. An expression product can be characterized as intracellular, extracellular or transmembrane.

The terms “treat” or “treatment” refer to therapeutic treatment wherein the object is to slow down or lessen an undesired physiological change or disease, or provide a beneficial or desired clinical outcome during treatment. Beneficial or desired clinical outcomes include alleviation of symptoms, diminishment of extent of disease, stabilization (i.e., a cessation in the worsening) of the state of disease, delay or slowing of disease progression, amelioration or palliation of the disease state, and/or remission (whether partial or total and whether detectable or undetectable). “Treatment” can also mean prolonging survival as compared to expected survival if a subject was not receiving treatment. Those in need of treatment include those subjects already with the undesired physiological change or disease as well as those subjects prone to having the physiological change or disease. Treatment may involve a treatment agent, also referred to herein as a “medicament” or “medication,” that may be intended to help achieve the beneficial or desired clinical outcome of interest by its action. Treatment agents or medicaments may be administered to a subject by many routes, including at least intravenous and oral routes. The term “intravenous,” in connection to the administration of treatment agents or medicaments, refers to the administration of said treatment agents or medicaments within one or more veins. The term “oral,” in connection to the administration of treatment agents or medicaments, refers to the administration of said treatment agents or medicaments via an oral passage such as the mouth.

As used herein, the term “subject” refers to an animal. The terms “subject” and “patient” may be used interchangeably herein in reference to a subject. As such, a “subject” includes a human that is being treated for a disease, or prevention of a disease, as a patient. The methods described herein may be used to treat an animal subject belonging to any classification. Examples of such animals include mammals. Mammals, include, but are not limited to, mammals of the order Rodentia, such as mice and hamsters, and mammals of the order Logomorpha, such as rabbits. The mammals may be of the order Carnivora, including felines (cats) and canines (dogs). The mammals may be of the order Artiodactyla, including bovines (cows) and swines (pigs) or of the order Perssodactyla, including equines (horses). The mammals may be of the order Primates, Ceboids, or Simoids (monkeys) or of the order Anthropoids (humans and apes). In some embodiments, the mammal is a human.

The term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The phrase “pharmaceutically acceptable”, as used in connection with compositions described herein, refers to molecular entities and other ingredients of such compositions that are physiologically tolerable and do not typically produce untoward reactions when administered to a mammal (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

The term “line of therapy,” as used in connection with methods of treatment herein, refers to one or more cycles of a planned treatment program, which may have consisted of one or more planned cycles of single-agent therapy or combination therapy, as well as a sequence of treatments administered in a planned manner. For example, a planned treatment approach of induction therapy followed by autologous stem cell transplantation followed by maintenance is one line of therapy. A new line of therapy is considered to have started when a planned course of therapy has been modified to include other treatment agents or medicaments (alone or in combination) as a result of disease progression, relapse, or toxicity. A new line of therapy is also considered to have started when a planned period of observation off therapy had been interrupted by a need for additional treatment for the disease.

The term “refractory,” as used in connection to treatment with a particular treatment agent or medicament or line of therapy herein, refers to diseases or disease subjects that fail to respond to said treatment agent or medicament or line of therapy. The phrase “refractory myeloma” refers to multiple myeloma that is nonresponsive while on primary or salvage therapy or that has progressed within 60 days of last therapy.

The phrase “nonresponsive disease” refers to either failure to achieve minimal response or to development of progressive disease while on therapy.

By “enhance” or “promote,” or “increase” or “expand” or “improve” refers generally to the ability of a composition contemplated herein to produce, elicit, or cause a greater physiological response (i.e., downstream effects) compared to the response caused by either vehicle or a control molecule/composition. A measurable physiological response may include an increase in T cell expansion, activation, effector function, persistence, and/or an increase in cancer cell death killing ability, among others apparent from the understanding in the art and the description herein. In certain embodiments, an “increased” or “enhanced” amount can be a “statistically significant” amount, and may include an increase that is 1.1, 1.2, 1.5, 2, 3, 4, 5, 6, 7, 8, 9, 10, 15, 20, 30, or more times (e.g., 500, 1000 times) (including all integers and decimal points in-between and above 1, e.g., 1.5, 1.6, 1.7. 1.8, etc.) the response produced by vehicle or a control composition.

By “decrease” or “lower,” or “lessen,” or “reduce,” or “abate” refers generally to the ability of composition contemplated herein to produce, elicit, or cause a lesser physiological response (i.e., downstream effects) compared to the response caused by either vehicle or a control molecule/composition. In certain embodiments, a “decrease” or “reduced” amount can be a “statistically significant” amount, and may include a decrease that is 1.1, 1.2, 1.5, 2, 3, 4, 5, 6, 7, 8, 9, 10, 15, 20, 30 or more times (e.g., 500, 1000 times) (including all integers and decimal points in-between and above 1, e.g., 1.5, 1.6, 1.7. 1.8, etc.) the response (reference response) produced by vehicle, a control composition, or the response in a particular cell lineage.

The terminology used herein is for the purpose of describing particular embodiments only and is not intended to be limiting. As used herein, the indefinite articles “a”, “an” and “the” should be understood to include plural reference unless the context clearly indicates otherwise.

Throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

### 4.2. Vectors

Polynucleotide sequences encoding the CARs described in the present application can be obtained using standard recombinant techniques. Desired polynucleotide sequences may be isolated and sequenced from antibody producing cells such as hybridoma cells. Alternatively, polynucleotides can be synthesized using nucleotide synthesizers or PCR techniques.

The disclosure also provides a vector comprising the nucleic acid sequence encoding the CAR disclosed herein. The vector can be, for example, a plasmid, a cosmid, a viral vector (e.g., retroviral or adenoviral), or a phage. Suitable vectors and methods of vector preparation are well known in the art (see, e.g., Sambrook et al. and Ausubel et al.).

In addition to the nucleic acid sequence encoding the CAR disclosed herein, the vector preferably comprises expression control sequences, such as promoters, enhancers, polyadenylation signals, transcription terminators, internal ribosome entry sites (IRES), and the like, that provide for the expression of the nucleic acid sequence in a host cell. Exemplary expression control sequences are known in the art and described in, for example, Goeddel, Gene Expression Technology: Methods in Enzymology, Vol. 185, Academic Press, San Diego, Calif. (1990).

In some embodiments, the vector comprises a promoter. A large number of promoters recognized by a variety of potential host cells are well known. The selected promoter can be operably linked to cistron DNA encoding the CAR disclosed herein by removing the promoter from the source DNA via restriction enzyme digestion and inserting the isolated promoter sequence into the vector of the present application. A large number of promoters, including constitutive, inducible, and repressible promoters, from a variety of different sources are well known in the art. Representative sources of promoters include for example, virus, mammal, insect, plant, yeast, and bacteria, and suitable promoters from these sources are readily available, or can be made synthetically, based on sequences publicly available, for example, from depositories such as the ATCC as well as other commercial or individual sources. Promoters can be unidirectional (i.e., initiate transcription in one direction) or bi-directional (i.e., initiate transcription in either a 3′ or 5′ direction). Non-limiting examples of promoters include, for example, the T7 bacterial expression system, pBAD (araA) bacterial expression system, the cytomegalovirus (CMV) promoter, the SV40 promoter, and the RSV promoter. Inducible promoters include, for example, the Tet system (U.S. Pat. Nos. 5,464,758 and 5,814,618), the Ecdysone inducible system (No et al., Proc. Natl. Acad. Sci., 93: 3346-3351 (1996)), the T-REX™ system (Invitrogen, Carlsbad, CA), LACSWITCH™ System (Stratagene, San Diego, CA), and the Cre-ERT tamoxifen inducible recombinase system (Indra et al., Nuc. Acid. Res., 27: 4324-4327 (1999); Nuc. Acid. Res., 28: e99 (2000); U.S. Pat. No. 7,112,715; and Kramer & Fussenegger, Methods Mol. Biol, 308: 123-144 (2005)).

In some embodiments, the vector comprises an “enhancer.” The term “enhancer” as used herein, refers to a DNA sequence that increases transcription of, for example, a nucleic acid sequence to which it is operably linked. Enhancers can be located many kilobases away from the coding region of the nucleic acid sequence and can mediate the binding of regulatory factors, patterns of DNA methylation, or changes in DNA structure. A large number of enhancers from a variety of different sources are well known in the art and are available as or within cloned polynucleotides (for e.g., from depositories such as the ATCC as well as other commercial or individual sources). A number of polynucleotides comprising promoters (such as the commonly used CMV promoter) also comprise enhancer sequences. Enhancers can be located upstream, within, or downstream of coding sequences. The term “Ig enhancers” refers to enhancer elements derived from enhancer regions mapped within the immunoglobulin (Ig) locus. Such Ig enhancers include, for example, the heavy chain (mu) 5′ enhancers, light chain (kappa) 5′ enhancers, kappa and mu intronic enhancers, and 3′ enhancers (see generally Paul W. E. (ed), Fundamental Immunology, 3rd Edition, Raven Press, New York (1993), pages 353-363; and U.S. Pat. No. 5,885,827).

In some embodiments, the vector comprises a “selectable marker gene.” The term “selectable marker gene”, as used herein, refers to a nucleic acid sequence that allows cells expressing the nucleic acid sequence to be specifically selected for or against, in the presence of a corresponding selective agent. Suitable selectable marker genes are known in the art and described in, for e.g., International Patent Application Publications WO 1992/08796 and WO 1994/28143; Wigler et al., Proc. Natl. Acad. Sci. USA, 77: 3567 (1980); O'Hare et al., Proc. Natl. Acad. Sci. USA, 78: 1527 (1981); Mulligan & Berg, Proc. Natl. Acad. Sci. USA, 78: 2072 (1981); Colberre-Garapin et al., J. Mol. Biol., 150: 1 (1981); Santerre et al., Gene, 30: 147 (1984); Kent et al., Science, 237: 901-903 (1987); Wigler et al., Cell, IP. 223 (1977); Szybalska & Szybalski, Proc. Natl. Acad. Sci. USA, 48: 2026 (1962); Lowy et al., Cell, 22: 817 (1980); and U.S. Pat. Nos. 5,122,464 and 5,770,359.

In some embodiments, the vector is an “episomal expression vector” or “episome,” which is able to replicate in a host cell, and persists as an extrachromosomal segment of DNA within the host cell in the presence of appropriate selective pressure (see, e.g., Conese et al., Gene Therapy, 11: 1735-1742 (2004)). Representative commercially available episomal expression vectors include, but are not limited to, episomal plasmids that utilize Epstein Barr Nuclear Antigen 1 (EBNA1) and the Epstein Barr Virus (EBV) origin of replication (oriP). The vectors pREP4, pCEP4, pREP7, and pcDNA3.1 from Invitrogen (Carlsbad, CA) and pB-CMV from Stratagene (La Jolla, CA) represent non-limiting examples of an episomal vector that uses T-antigen and the SV40 origin of replication in lieu of EBNA1 and oriP.

In some embodiments, the vector is an “integrating expression vector,” which may randomly integrate into the host cell's DNA or may include a recombination site to enable recombination between the expression vector and a specific site in the host cell's chromosomal DNA. Such integrating expression vectors may utilize the endogenous expression control sequences of the host cell's chromosomes to effect expression of the desired protein. Examples of vectors that integrate in a site specific manner include, for example, components of the flp-in system from Invitrogen (Carlsbad, CA) (e.g., pcDNA™5/FRT), or the cre-lox system, such as can be found in the pExchange-6 Core Vectors from Stratagene (La Jolla, CA). Examples of vectors that randomly integrate into host cell chromosomes include, for example, pcDNA3.1 (when introduced in the absence of T-antigen) from Invitrogen (Carlsbad, CA), and pCI or pFNI OA (ACT) FLEXI™ from Promega (Madison, WI).

In some embodiments, the vector is a viral vector. Representative viral expression vectors include, but are not limited to, the adenovirus-based vectors (e.g., the adenovirus-based Per.C6 system available from Crucell, Inc. (Leiden, The Netherlands)), lentivirus-based vectors (e.g., the lentiviral-based pLPl from Life Technologies (Carlsbad, CA)), and retrovirus-based vectors (e.g., the pFB-ERV plus pCFB-EGSH from Stratagene (La Jolla, CA)). In a preferred embodiment, the vector is a lentiviral vector.

In some embodiments, the vector comprising the nucleic acid encoding the CAR disclosed herein is introduced into a host cell that is capable of containing a heterologous nucleic acid. As used herein, the term “host cell” means any cell that contains a heterologous nucleic acid. The heterologous nucleic acid can be a vector (e.g., an expression vector). For example, a host cell can be a cell from any organism that is selected, modified, transformed, grown, used or manipulated in any way, for the production of a substance by the cell, for example the expression by the cell of a gene, a DNA or RNA sequence, a protein or an enzyme. An appropriate host may be determined. For example, the host cell may be selected based on the vector backbone and the desired result. By way of example, a plasmid or cosmid can be introduced into a prokaryote host cell for replication of several types of vectors. Bacterial cells such as, but not limited to DH55α, JM109, and KCB, SURE® Competent Cells, and SOLOPACK Gold Cells, can be used as host cells for vector replication and/or expression. Additionally, bacterial cells such as E. coli LE392 could be used as host cells for phage viruses. Eukaryotic cells that can be used as host cells include, but are not limited to yeast (e.g., YPH499, YPH500 and YPH501), insects and mammals. Examples of mammalian eukaryotic host cells for replication and/or expression of a vector include, but are not limited to, HeLa, NIH3T3, Jurkat, 293, COS, CHO, Saos, and PC12. Preferably, the host cell is a cell that can contain the expression vector. In preferred embodiments, host cells are those that can be easily and reliably grown, have reasonably fast growth rates, have well characterized expression systems, and can be transformed or transfected easily and efficiently. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5α E. coli cells, Chinese hamster ovarian cells, monkey VERO cells, COS cells, HEK 293 cells, and the like. In a preferred embodiment, the host cells are HEK 293 cells. In some embodiments, the HEK 293 cells are derived from the ATCC SD-3515 line. In some embodiments, the HEK 293 cells are derived from, the IU-VPF MCB line. In some embodiments, the HEK 293 cells are derived from the IU-VPF MWCB line. In some embodiments, the host cell can be a peripheral blood lymphocyte (PBL), a peripheral blood mononuclear cell (PBMC), or a natural killer (NK). Preferably, the host cell is a natural killer (NK) cell. More preferably, the host cell is a T-cell.

For purposes of amplifying or replicating the recombinant expression vector, the host cell may be a prokaryotic cell, e.g., a DH5α cell. For purposes of producing a virus from a viral expression vector, the host cell may be a eukaryotic cell, e.g., a HEK 293 cell. For purposes of producing a recombinant CAR, the host cell can be a mammalian cell. The mammalian host cell preferably is a human cell. The host cell can be of any cell type, can originate from any type of tissue, and can be of any developmental stage. Methods for selecting suitable mammalian host cells and methods for transformation, culture, amplification, screening, and purification of cells are known in the art.

In some embodiments, the disclosure provides an isolated host cell which expresses the nucleic acid sequence encoding the CAR described herein.

In some embodiments, the host cell is a T-cell. The T-cell of the disclosure can be any T-cell, such as a cultured T-cell, e.g., a primary T-cell, or a T-cell from a cultured T-cell line, or a T-cell obtained from a mammal. If obtained from a mammal, the T-cell can be obtained from numerous sources, including but not limited to blood, bone marrow, lymph node, the thymus, or other tissues or fluids. T-cells can also be enriched for or purified. The T-cell preferably is a human T-cell (e.g., isolated from a human). The T-cell can be of any developmental stage, including but not limited to, a CD4+/CD8+ double positive T-cell, a CD4+ helper T-cell, e.g., Th, and Th2 cells, a CD8+ T-cell (e.g., a cytotoxic T-cell), a tumor infiltrating cell, a memory T-cell, a naive T-cell, and the like. In one embodiment, the T-cell is a CD8+ T-cell or a CD4+ T-cell. T-cell lines are available from, e.g., the American Type Culture Collection (ATCC, Manassas, VA), and the German Collection of Microorganisms and Cell Cultures (DSMZ) and include, for example, Jurkat cells (ATCC TIB-152), Sup-Tl cells (ATCC CRL-1942), RPMI 8402 cells (DSMZ ACC-290), Karpas 45 cells (DSMZ ACC-545), and derivatives thereof.

In some embodiments, the host cell is a natural killer (NK) cell. NK cells are a type of cytotoxic lymphocyte that plays a role in the innate immune system. NK cells are defined as large granular lymphocytes and constitute a third kind of cells differentiated from the common lymphoid progenitor which also gives rise to B and T lymphocytes (see, e.g., Immunobiology, 5th ed., Janeway et al., eds., Garland Publishing, New York, NY (2001)). NK cells differentiate and mature in the bone marrow, lymph node, spleen, tonsils, and thymus. Following maturation, NK cells enter into the circulation as large lymphocytes with distinctive cytotoxic granules. NK cells are able to recognize and kill some abnormal cells, such as, for example, some tumor cells and virus-infected cells, and are thought to be important in the innate immune defense against intracellular pathogens. As described above with respect to T-cells, the NK cell can be any NK cell, such as a cultured NK cell, e.g., a primary NK cell, or an NK cell from a cultured NK cell line, or an NK cell obtained from a mammal. If obtained from a mammal, the NK cell can be obtained from numerous sources, including but not limited to blood, bone marrow, lymph node, the thymus, or other tissues or fluids. NK cells can also be enriched for or purified. The NK cell preferably is a human NK cell (e.g., isolated from a human). NK cell lines are available from, e.g., the American Type Culture Collection (ATCC, Manassas, VA) and include, for example, NK-92 cells (ATCC CRL-2407), NK92MI cells (ATCC CRL-2408), and derivatives thereof.

The nucleic acid sequence encoding a CAR disclosed herein may be introduced into a cell by “transfection”, “transformation”, or “transduction”. “Transfection”, “transformation”, or “transduction”, as used herein, refer to the introduction of one or more exogenous polynucleotides into a host cell by using physical or chemical methods.

The term “transformation” means the introduction of one or more exogenous polynucleotides into bacterial cells that have been made competent for transformation, for e.g., by use of dimethylsulfoxide, divalent cations such as calcium, or polyethylene glycol. Many transformation techniques are known in the art and include heat shock and electric shock.

The term “transfection” means the introduction of a “foreign” (i.e., extrinsic or extracellular) nucleic acid into a cell using recombinant DNA technology. The term “genetic modification” means the introduction of a “foreign” (i.e., extrinsic or extracellular) gene, DNA or RNA sequence to a host cell, so that the host cell will express the introduced gene or sequence to produce a desired substance, typically a protein or enzyme coded by the introduced gene or sequence. The introduced gene or sequence may also be called a “cloned” or “foreign” gene or sequence, may include regulatory or control sequences operably linked to polynucleotide encoding the chimeric antigen receptor, such as start, stop, promoter, signal, secretion, or other sequences used by a cell's genetic machinery. The gene or sequence may include nonfunctional sequences or sequences with no known function. A host cell that receives and expresses introduced DNA or RNA has been “genetically engineered.” The DNA or RNA introduced to a host cell can come from any source, including cells of the same genus or species as the host cell, or from a different genus or species. Many transfection techniques are known in the art and include, for example, calcium phosphate DNA co-precipitation (see, e.g., Murray E. J. (ed.), Methods in Molecular Biology, Vol. 7, Gene Transfer and Expression Protocols, Humana Press (1991)); DEAE-dextran; electroporation; cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

The term “transduction” means the introduction of a foreign nucleic acid into a cell using a viral vector. Phage or viral vectors can be introduced into host cells via transduction by infectious viral particles. Said infectious viral particles may be grown in suitable packaging cells, many of which are commercially available and known in the art.

The term “regulatory element” refers to any cis-acting genetic element that controls some aspect of the expression of nucleic acid sequences. In some embodiments, the term “promoter” comprises essentially the minimal sequences required to initiate transcription. In some embodiments, the term “promoter” includes the sequences to start transcription, and in addition, also include sequences that can upregulate or downregulate transcription, commonly termed “enhancer elements” and “repressor elements”, respectively.

### 4.3. Antibodies and Derived Proteins

Suitable methods of making antibodies are known in the art. For instance, standard hybridoma methods are described in, e.g., Kohler and Milstein, Eur. J. Immunol., 5, 511-519 (1976), Harlow and Lane (eds.), Antibodies: A Laboratory Manual, CSH Press (1988), and C. A. Janeway et al. (eds.), Immunobiology, 5th Ed., Garland Publishing, New York, N.Y. (2001)). Alternatively, other methods, such as EBV-hybridoma methods (Haskard and Archer, J. Immunol. Methods, 74(2), 361-67 (1984), and Roder et al., Methods Enzymol., 121, 140-67 (1986)), and bacteriophage vector expression systems (see, e.g., Huse et al., Science, 246, 1275-81 (1989)) are known in the art. Further, methods of producing antibodies in non-human animals are described in, e.g., U.S. Pat. Nos. 5,545,806, 5,569,825, and 5,714,352, and U.S. Patent Application Publication No. 2002/0197266 A1).

Phage display can also be used to generate an antibody. In this regard, phage libraries encoding antigen-binding variable (V) domains of antibodies can be generated using standard molecular biology and recombinant DNA techniques (see, e.g., Sambrook et al., supra, and Ausubel et al., supra). Phage encoding a variable region with the desired specificity are selected for specific binding to the desired antigen, and a complete or partial antibody is reconstituted comprising the selected variable domain. Nucleic acid sequences encoding the reconstituted antibody are introduced into a suitable cell line, such as a myeloma cell used for hybridoma production, such that antibodies having the characteristics of monoclonal antibodies are secreted by the cell (see, e.g., Janeway et al., supra, Huse et al., supra, and U.S. Pat. No. 6,265,150).

The antibodies, polypeptides, and proteins of embodiments of the disclosure (including functional portions and functional variants) can be subject to post-translational modifications. They can be glycosylated, esterified, N-acylated, amidated, carboxylated, phosphorylated, esterified, cyclized via, e.g., a disulfide bridge, or converted into an acid addition salt. In some embodiments, they are dimerized or polymerized, or conjugated.

The antibodies, polypeptides, and/or proteins of embodiments of the disclosure (including functional portions and functional variants thereof) can be obtained by methods known in the art. Suitable methods of de novo synthesizing polypeptides and proteins are described in references, such as Chan et al., Fmoc Solid Phase Peptide Synthesis, Oxford University Press, Oxford, United Kingdom, 2000; Peptide and Protein Drug Analysis, ed. Reid, R., Marcel Dekker, Inc., 2000; and Epitope Mapping, ed. Westwood et al., Oxford University Press, Oxford, United Kingdom, 2001. Also, polypeptides and proteins can be recombinantly produced using the nucleic acids described herein using standard recombinant methods. See, for instance, Sambrook et al., Molecular Cloning: A Laboratory Manual, 3rd ed., Cold Spring Harbor Press, Cold Spring Harbor, N.Y. 2001; and Ausubel et al., Current Protocols in Molecular Biology, Greene Publishing Associates and John Wiley & Sons, NY, 1994. Further, some of the antibodies, polypeptides, and proteins of the disclosure (including functional portions and functional variants thereof) can be isolated and/or purified from a source, such as a plant, a bacterium, an insect, a mammal, etc. Methods of isolation and purification are known in the art. Alternatively, the antibodies, polypeptides, and/or proteins described herein (including functional portions and functional variants thereof) can be commercially synthesized. In this respect, the antibodies, polypeptides, and proteins can be synthetic, recombinant, isolated, and/or purified.

### 4.4. Chimeric Antigen Receptors

International Patent Publication No. WO 2018/028647 is incorporated by reference herein in its entirety. US Patent Publication No. 2018/0230225 is incorporated by reference herein in its entirety. International Patent Application No. PCT/CN2020/133598 is incorporated by reference herein in its entirety.

The disclosure provides for methods of treating a subject with cells expressing a chimeric antigen receptor (CAR). The CAR comprises an extracellular antigen binding domain comprising one or more single-domain antibodies. In various embodiments, there is provided a CAR targeting BCMA (also referred herein as “BCMA CAR”) comprising a polypeptide comprising: (a) an extracellular antigen binding domain comprising an anti-BCMA binding moiety; (b) a transmembrane domain; and (c) an intracellular signaling domain. In some embodiments, the anti-BCMA binding moiety is camelid, chimeric, human, or humanized. In some embodiments, the intracellular signaling domain comprises a primary intracellular signaling domain of an immune effector cell (such as T cell). In some embodiments, the primary intracellular signaling domain is derived from CD4. In some embodiments, the primary intracellular signaling domain is derived from CD3-zeta. In some embodiments, the intracellular signaling domain comprises a co-stimulatory signaling domain. In some embodiments, the co-stimulatory signaling domain is derived from a co-stimulatory molecule selected from the group consisting of CD27, CD28, CD137, OX40, CD30, CD40, CD3, LFA-1, ICOS, CD2, CD7, LIGHT, NKG2C, B7-H3, ligands of CD83 and combinations thereof. In certain embodiments, the co-stimulatory signaling domain is derived from CD137.

In some embodiments, the BCMA CAR further comprises a hinge domain (such as a CD8-alpha hinge domain) located between the C-terminus of the extracellular antigen binding domain and the N-terminus of the transmembrane domain. In some embodiments, the BCMA CAR further comprises a signal peptide (such as a CD8-alpha signal peptide) located at the N-terminus of the polypeptide. In some embodiments, the polypeptide comprises from the N-terminus to the C-terminus: a CD8-alpha signal peptide, the extracellular antigen-binding domain, a CD8-alpha hinge domain, a CD28 transmembrane domain, a first co-stimulatory signaling domain derived from CD28, a second co-stimulatory signaling domain derived from CD137, and a primary intracellular signaling domain derived from CD4. In some embodiments, the polypeptide comprises from the N-terminus to the C-terminus: a CD8-alpha signal peptide, the extracellular antigen-binding domain, a CD8-alpha hinge domain, a CD8-alpha transmembrane domain, a second co-stimulatory signaling domain derived from CD137, and a primary intracellular signaling domain derived from CD3-zeta. In some embodiments, the BCMA CAR is monospecific. In some embodiments, the BCMA CAR is monovalent.

The present application also provides CARs that have two or more (including, but not limited to, any one of 2, 3, 4, 5, 6, or more) binding moieties that specifically bind to an antigen, such as BCMA. In some embodiments, one or more of the binding moieties are antigen binding fragments. In some embodiments, one or more of the binding moieties comprise single-domain antibodies. In some embodiments, one or more of the binding moieties comprise a VHH.

In some embodiments, the CAR is a multivalent (such as bivalent, trivalent, or of higher number of valencies) CAR comprising a polypeptide comprising: (a) an extracellular antigen binding domain comprising a plurality (such as at least about any one of 2, 3, 4, 5, 6, or more) of binding moieties specifically binding to an antigen (such as a tumor antigen); (b) a transmembrane domain; and (c) an intracellular signaling domain.

In some embodiments, the binding moieties, such as VHHs (including the plurality of VHHs, or the first VHH and/or the second VHH) are camelid, chimeric, human, or humanized. In some embodiments, the binding moieties or VHHs are connected to each other via peptide bonds or peptide linkers. In some embodiments, each peptide linker is no more than about 50 (such as no more than about any one of 35, 25, 20, 15, 10, or 5) amino acids long.

In some embodiments, the first BCMA binding moiety and/or the second BCMA binding moiety is an anti-BCMA VHH. In some embodiments, the first BCMA binding moiety is a first anti-BCMA VHH and the second BCMA binding moiety is a second anti-BCMA VHH.

In some embodiments, the first anti-BCMA binding moiety comprises a first complementarity determining region (CDR1) comprising the amino acid sequence of SEQ ID NO: 18. In some embodiments, the first anti-BCMA binding moiety comprises a second complementarity determining region (CDR2) comprising the amino acid sequence of SEQ ID NO: 19. In some embodiments, the first anti-BCMA binding moiety comprises a third complementarity determining region (CDR3) comprising the amino acid sequence of SEQ ID NO: 20.

In some embodiments, the first anti-BCMA binding moiety comprises a first complementarity determining region (CDR1) comprising the amino acid sequence of SEQ ID NO: 18, a second complementarity determining region (CDR2) comprising the amino acid sequence of SEQ ID NO: 19, and a third complementarity determining region (CDR3) comprising the amino acid sequence of SEQ ID NO: 20.

In some embodiments, the first BCMA binding moiety comprises the amino acid sequence of SEQ ID NO: 2. In some embodiments, the first BCMA binding moiety comprises a polypeptide encoded by the nucleic acid sequence of SEQ ID NO: 10. In some embodiments, the first anti-BCMA binding moiety comprises one or more of, or all of, SEQ ID NO: 18, SEQ ID NO: 19, SEQ ID NO: 20 and SEQ ID NO: 2. These sequences correspond to the sequences present in cilta-cel.

In some embodiments, the second BCMA binding moiety comprises a first complementarity determining region (CDR1) comprising the amino acid sequence of SEQ ID NO: 21. In some embodiments, the second BCMA binding moiety comprises a second complementarity determining region (CDR2) comprising the amino acid sequence of SEQ ID NO: 22. In some embodiments, the second BCMA binding moiety comprises a third complementarity determining region (CDR3) comprising the amino acid sequence of SEQ ID NO: 23.

In some embodiments, the second BCMA binding moiety comprises a first complementarity determining region (CDR1) comprising the amino acid sequence of SEQ ID NO: 21, a second complementarity determining region (CDR2) comprising the amino acid sequence of SEQ ID NO: 22, and a third complementarity determining region (CDR3) comprising the amino acid sequence of SEQ ID NO: 23.

In some embodiments, the second BCMA binding moiety comprises the amino acid sequence of SEQ ID NO: 4. In some embodiments, the second BCMA binding moiety comprises a polypeptide encoded by the nucleic acid sequence of SEQ ID NO: 12. In some embodiments, the second anti-BCMA binding moiety comprises one or more of, or all of, SEQ ID NO: 21, SEQ ID NO: 22, SEQ ID NO: 23 and SEQ ID NO: 4. These sequences correspond to the sequences present in cilta-cel.

In some embodiments, the first BCMA binding moiety and the second BCMA binding moiety are connected to each other via a peptide linker. In some embodiments, the peptide linker comprises the amino acid sequence of SEQ ID NO: 3. In some embodiments, the peptide linker comprises a polypeptide encoded by the nucleic acid sequence of SEQ ID NO: 11.

In some embodiments, the CAR further comprises a hinge domain (such as a CD8-alpha hinge domain) located between the C-terminus of the extracellular antigen binding domain and the N-terminus of the transmembrane domain. In some embodiments, the CAR further comprises a signal peptide (such as a CD8-alpha signal peptide) located at the N-terminus of the polypeptide.

Without wishing to be bound by theory, the CARs that are multivalent, or those CARs comprising an extracellular antigen binding domain comprising a first BCMA binding moiety and a second BCMA binding moiety, may be specially suitable for targeting multimeric antigens via synergistic binding by the different antigen binding sites, or for enhancing binding affinity or avidity to the antigen. Improved avidity may allow for a substantial reduction in the dose of CAR-T cells needed to achieve a therapeutic effect, such as a dose ranging from 4.0×10⁴ to 1.0×10⁶ CAR-T cells per kilogram of the mass of the subject, or 3.0×10⁶ to 1.0×10⁸ total CAR-T expressing cells. Monovalent CARs, such as bb2121, may need to be dosed at 5 to 10 times these amounts to achieve a comparable effect. In various embodiments, reduced dosage ranges may provide for substantial reduction in cytokine release syndrome (CRS) and other potentially dangerous side-effects of CAR-T therapy.

The various binding moieties (e.g., an extracellular antigen binding domain comprising a first BCMA binding moiety and a second BCMA binding moiety) in the CARs described herein may be connected to each other via peptide linkers. The peptide linkers connecting different binding moieties (such as VHHs) may be the same or different. Different domains of the CARs may also be connected to each other via peptide linkers. In some embodiments, the binding moieties (such as VHHs) are directly connected to each other without any peptide linkers.

The peptide linker in the CARs described herein can be of any suitable length. In some embodiments, the peptide linker is at least about any of 1, 2, 3, 4, 5, 6, 7, 8, 9, 10, 11, 12, 13, 14, 15, 16, 17, 18, 19, 20, 25, 30, 35, 40, 50, 75, 100 or more amino acids long. In some embodiments, the peptide linker is no more than about any of 100, 75, 50, 40, 35, 30, 25, 20, 19, 18, 17, 16, 15, 14, 13, 12, 11, 10, 9, 8, 7, 6, 5 or fewer amino acids long. In some embodiments, the length of the peptide linker is any of about 1 amino acid to about 10 amino acids, about 1 amino acids to about 20 amino acids, about 1 amino acid to about 30 amino acids, about 5 amino acids to about 15 amino acids, about 10 amino acids to about 25 amino acids, about 5 amino acids to about 30 amino acids, about 10 amino acids to about 30 amino acids long, about 30 amino acids to about 50 amino acids, about 50 amino acids to about 100 amino acids, or about 1 amino acid to about 100 amino acids.

The CARs of the present application comprise a transmembrane domain that can be directly or indirectly connected to the extracellular antigen binding domain.

The CAR may comprise a T-cell activation moiety. The T-cell activation moiety can be any suitable moiety derived or obtained from any suitable molecule. In one embodiment, for example, the T-cell activation moiety comprises a transmembrane domain. The transmembrane domain can be any transmembrane domain derived or obtained from any molecule known in the art. For example, the transmembrane domain can be obtained or derived from a CD8α molecule or a CD28 molecule. Without wishing to be bound by theory, CD8 is a transmembrane glycoprotein that serves as a co-receptor for the T-cell receptor (TCR) and is expressed primarily on the surface of cytotoxic T-cells. The most common form of CD8 exists as a dimer composed of a CD8 alpha (CD8α) and CD8 beta (CD8β) chain. CD28 is expressed on T-cells and provides co-stimulatory signals required for T-cell activation. CD28 is the receptor for CD80 (B7.1) and CD86 (B7.2). In a preferred embodiment, the CD8a and CD28 are human.

In addition to the transmembrane domain, the T-cell activation moiety may further comprise an intracellular (i.e., cytoplasmic) T-cell signaling domain. The intercellular T-cell signaling domain can be obtained or derived from a CD28 molecule, a CD3 zeta (ζ) molecule or modified versions thereof, a human Fc receptor gamma (FcRy) chain, a CD27 molecule, an OX40 molecule, a 4-1BB molecule, or other intracellular signaling molecules known in the art. Without wishing to be bound by theory: (1) CD28 is a T-cell marker important in T-cell co-stimulation; (2) CD3ζ associates with TCRs to produce a signal and contains immunoreceptor tyrosine-based activation motifs (ITAMs); and (3) 4-1BB, also known as CD137, transmits a potent costimulatory signal to T-cells, promoting differentiation and enhancing long-term survival of T lymphocytes. In a preferred embodiment, the CD28, CD3 zeta, 4-IBB, OX40, and CD27 are human.

The T-cell activation domain of the CAR encoded by the nucleic acid sequence disclosed herein can comprise any one of aforementioned transmembrane domains and any one or more of the aforementioned intercellular T-cell signaling domains in any combination. For example, the nucleic acid sequence disclosed herein can encode a CAR comprising a CD28 transmembrane domain and intracellular T-cell signaling domains of CD28 and CD3 zeta. Alternatively, for example, the nucleic acid sequence disclosed herein can encode a CAR comprising a CD8a transmembrane domain and intracellular T-cell signaling domains of CD28, CD3 zeta, the Fc receptor gamma (FcRy) chain, and/or 4-1 BB.

In some embodiments, the CAR polypeptide further comprises a signal peptide located at the N-terminus of the polypeptide. In some embodiments, the signal peptide is derived from CD8-alpha (CD8a SP). In some embodiments, the signal peptide comprises the amino acid sequence of SEQ ID NO: 1. In some embodiments, the signal peptide comprises a polypeptide encoded by the nucleic acid sequence of SEQ ID NO: 9.

In some embodiments, the transmembrane domain comprises the amino acid sequence of SEQ ID NO: 6. In some embodiments, the transmembrane domain comprises a polypeptide encoded by the nucleic acid sequence of SEQ ID NO: 14.

In some embodiments, the intracellular signaling domain comprises a primary intracellular signaling domain of an immune effector cell. In some embodiments, the intracellular signaling domain is derived from CD3ζ. In some embodiments, the intracellular signaling domain comprises at least one co-stimulatory signaling domains. In some embodiments, the intracellular signaling domain comprises the amino acid sequence of SEQ ID NO: 8. In some embodiments, the intracellular signaling domain comprises a polypeptide encoded by the nucleic acid sequence of SEQ ID NO: 16. In some embodiments, the intracellular signaling domain comprises an amino acid sequence of SEQ ID NO: 7. In some embodiments, the intracellular signaling domain comprises a polypeptide encoded by the nucleic acid sequence of SEQ ID NO: 15.

In some embodiments, the CAR polypeptide further comprises a hinge domain located between the C-terminus of the extracellular antigen binding domain and the N-terminus of the transmembrane domain. In some embodiments, the hinge domain comprises the amino acid sequence of SEQ ID NO: 5. In some embodiments, the hinge domain comprises a polypeptide encoded by the nucleic acid sequence of SEQ ID NO: 13.

In some embodiments, the CAR comprises a first and a second anti-BCMA binding moiety, wherein the first anti-BCMA binding moiety comprises a first complementarity determining region (CDR1) comprising the amino acid sequence of SEQ ID NO: 18, a second complementarity determining region (CDR2) comprising the amino acid sequence of SEQ ID NO: 19, and a third complementarity determining region (CDR3) comprising the amino acid sequence of SEQ ID NO: 20; wherein the second BCMA binding moiety comprises a first complementarity determining region (CDR1) comprising the amino acid sequence of SEQ ID NO: 21, a second complementarity determining region (CDR2) comprising the amino acid sequence of SEQ ID NO: 22, and a third complementarity determining region (CDR3) comprising the amino acid sequence of SEQ ID NO: 23; wherein the CAR further comprises: a transmembrane domain derived from CD8a, wherein optionally the transmembrane domain comprises the amino acid sequence of SEQ ID NO: 6; a primary intracellular signaling domain derived from CD3ζ, wherein optionally the primary intracellular signaling domain comprises the amino acid sequence of SEQ ID NO: 8; a co-stimulatory signaling domain comprising a cytoplasmic domain of CD137, wherein optionally the co-stimulatory signaling domain comprises the amino acid sequence of SEQ ID NO: 7; and a hinge domain located between the C-terminus of the extracellular antigen binding domain and the N-terminus of the transmembrane domain, wherein the hinge domain is derived from CD8α, wherein optionally the hinge domain comprises the amino acid sequence of SEQ ID NO: 5. In certain such embodiments, the first VHH domain comprises the amino acid sequence of SEQ ID NO: 2 and the second VHH domain comprises the amino acid sequence of SEQ ID NO: 4.

In some embodiments, the CAR comprises one or more of, or all of, SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 18, SEQ ID NO: 19, SEQ ID NO: 20, SEQ ID NO: 21, SEQ ID NO: 22 and SEQ ID NO: 23. In one embodiments, the CAR comprises SEQ ID NO: 17. In some embodiments, the CAR comprises a polypeptide encoded by the nucleic acid sequence of one or more of, or all of, SEQ ID NO: 9, SEQ ID NO: 10, SEQ ID NO: 11, SEQ ID NO: 12, SEQ ID NO: 13, SEQ ID NO: 14, SEQ ID NO: 15 and SEQ ID NO: 16.

### 4.5. Immune Effector Cell Compositions

“Immune effector cells” are immune cells that can perform immune effector functions. In some embodiments, the immune effector cells express at least FcγRIII and perform ADCC effector function. Examples of immune effector cells which mediate ADCC include peripheral blood mononuclear cells (PBMC), natural killer (NK) cells, monocytes, cytotoxic T cells, neutrophils, and eosinophils. In some embodiments, the immune effector cells are T cells. In some embodiments, the T cells are autologous T cells. In some embodiments, the T cells are allogeneic T cells. In some embodiments, the T cells are CD4+/CD8−, CD4−/CD8+, CD4+/CD8+, CD4−/CD8−, or combinations thereof. In some embodiments, the T cells produce IL-2, TFN, and/or TNF upon expressing the CAR and binding to the target cells, such as CD20+ or CD19+ tumor cells. In some embodiments, the CD8+ T cells lyse antigen-specific target cells upon expressing the CAR and binding to the target cells.

Biological methods for introducing the vector into an immune effector cell include the use of DNA and RNA vectors. Viral vectors have become the most widely used method for inserting genes into mammalian, e.g., human cells. Chemical means for introducing the vector into an immune effector cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro is a liposome (e.g., an artificial membrane vesicle).

Provided herein are dosage forms comprising 3.0×10⁷ to 1.0×10⁸ CAR-T cells comprising a CAR comprising a polypeptide provided herein. Provided herein are dosage forms comprising 3.0×10⁷ to 1.0×10⁸ CAR-T cells comprising a CAR comprising a polypeptide comprising: (a) an extracellular antigen binding domain comprising a first BCMA binding moiety specifically binding to a first epitope of BCMA, and a second BCMA binding moiety specifically binding to a second epitope of BCMA; (b) a transmembrane domain; and (c) an intracellular signaling domain, wherein the first epitope and the second epitope are different. In some embodiments, there are provided dosage forms comprising 3.0×10⁷ to 1.0×10⁸ engineered immune effector cells (such as T-cells) comprising a CAR comprising a polypeptide comprising: (a) an extracellular antigen binding domain comprising a first anti-BCMA VHH specifically binding to a first epitope of BCMA, and a second anti-BCMA VHH specifically binding to a second epitope of BCMA; (b) a transmembrane domain; and (c) an intracellular signaling domain, wherein the first epitope and the second epitope are different. In certain embodiments, the dosage form comprises 3.0×10⁷ to 4.0×10⁷ of the CAR-T cells. In certain embodiments, the dosage form comprises 3.5×10⁷ to 4.5×10⁷ of the CAR-T cells. In certain embodiments, the dosage form comprises 4.0×10⁷ to 5.0×10⁷ of the CAR-T cells. In certain embodiments, the dosage form comprises 4.5×10⁷ to 5.5×10⁷ of the CAR-T cells. In certain embodiments, the dosage form comprises 5.0×10⁷ to 6.0×10⁷ of the CAR-T cells. In certain embodiments, the dosage form comprises 5.5×10⁷ to 6.5×10⁷ of the CAR-T cells. In certain embodiments, the dosage form comprises 6.0×10⁷ to 7.0×10⁷ of the CAR-T cells. In certain embodiments, the dosage form comprises 6.5×10⁷ to 7.5×10⁷ of the CAR-T cells. In certain embodiments, the dosage form comprises 7.0×10⁷ to 8.0×10⁷ of the CAR-T cells. In certain embodiments, the dosage form comprises 7.5×10⁷ to 8.5×10⁷ of the CAR-T cells. In certain embodiments, the dosage form comprises 8.0×10⁷ to 9.0×10⁷ of the CAR-T cells. In certain embodiments, the dosage form comprises 8.5×10⁷ to 9.5×10⁷ of the CAR-T cells. In certain embodiments, the dosage form comprises 9.0×10⁷ to 1.0×10⁸ of the CAR-T cells.

In some embodiments, the cell population of the CAR-T dosage forms described herein comprise a T cell or population of T cells, e.g., at various stages of differentiation. Stages of T cell differentiation include naive T cells, stem central memory T cells, central memory T cells, effector memory T cells, and terminal effector T cells, from least to most differentiated. After antigen exposure, naïve T cells proliferate and differentiate into memory T cells, e.g., stem central memory T cells and central memory T cells, which then differentiate into effector memory T cells. Upon receiving appropriate T cell receptor, costimulatory, and inflammatory signals, memory T cells further differentiate into terminal effector T cells. See, e.g., Restifo. Blood. 124.4(2014):476-77; and Joshi et al. J. Immunol. 180.3(2008):1309-15.

Naïve T cells can have the following expression pattern of cell surface markers: CCR7+, CD62L+, CD45RO−, CD95−. Stem central memory T cells (Tscm) can have the following expression pattern of cell surface markers: CCR7+, CD62L+, CD45RO−, CD95+. Central memory T cells (Tcm) can have the following expression pattern of cell surface markers: CCR7+, CD62L+, CD45RO+, CD95+. Effector memory T cells (Tem) can have the following expression pattern of cell surface markers: CCR7−, CD62L−, CD45RO+, CD95+. Terminal effector T cells (Teff) can have the following expression pattern of cell surface markers: CCR7−, CD62L−, CD45RO−, CD95+. See, e.g., Gattinoni et al. Nat. Med. 17(2011):1290-7; and Flynn et al. Clin. Translat. Immunol. 3(2014):e20.

### 4.6. Pharmaceutical Compositions and Formulations

Further provided by the present application are pharmaceutical compositions comprising any one of the anti-BCMA antibodies of the disclosure, or any one of the engineered immune effector cells comprising any one of the CARs (such as BCMA CARs) as described herein, and a pharmaceutically acceptable carrier. Pharmaceutical compositions can be prepared by mixing any of the immune effector cells described herein, having the desired degree of purity, with optional pharmaceutically acceptable carriers, excipients or stabilizers (Remington's Pharmaceutical Sciences 16th edition, Osol, A. Ed. (1980)), in the form of lyophilized formulations or aqueous solutions. In certain embodiments, a pharmaceutical composition of CAR-T cells further comprises an excipient selected from dimethylsulfoxide or dextran-40.

The compositions described herein may be administered as part of a pharmaceutical composition comprising one or more carriers. The choice of carrier will be determined in part by the particular nucleic acid sequence, vector, or host cells expressing the CAR disclosed herein, as well as by the particular method used to administer the nucleic acid sequence, vector, or host cells expressing the CAR disclosed herein. Accordingly, there are a variety of suitable formulations of the pharmaceutical composition of the disclosure.

For example, the pharmaceutical composition can contain preservatives. Suitable preservatives may include, for example, methylparaben, propylparaben, sodium benzoate, and benzalkonium chloride. A mixture of two or more preservatives optionally may be used. The preservative or mixtures thereof are typically present in an amount of about 0.0001% to about 2% by weight of the total composition.

In addition, buffering agents may be used in the composition. Suitable buffering agents include, for example, citric acid, sodium citrate, phosphoric acid, potassium phosphate, and various other acids and salts. A mixture of two or more buffering agents optionally may be used. The buffering agent or mixtures thereof are typically present in an amount of about 0.001% to about 4% by weight of the total composition.

The composition comprising the nucleic acid sequence encoding the CAR disclosed herein, or host cells expressing the CAR disclosed herein, can be formulated as an inclusion complex, such as cyclodextrin inclusion complex, or as a liposome. Liposomes can serve to target the host cells (e.g., T-cells or NK cells) or the nucleic acid sequence disclosed herein to a particular tissue. Liposomes also can be used to increase the half-life of the nucleic acid sequence disclosed herein. Many methods are available for preparing liposomes, such as those described in, for example, Szoka et al., Ann. Rev. Biophys. Bioeng., 9: 467 (1980), and U.S. Pat. Nos. 4,235,871; 4,501,728; 4,837,028; and 5,019,369. The composition can employ time-released, delayed release, and sustained release delivery systems such that the delivery of the composition disclosed herein occurs prior to, and with sufficient time to cause, sensitization of the site to be treated. Many types of release delivery systems are available and known to those of ordinary skill in the art. Such systems can avoid repeated administrations of the composition, thereby increasing convenience to the subject and the physician, and may be particularly suitable for certain composition embodiments of the disclosure.

In certain embodiments, the CAR-T cells are formulated at a dose of about 1.0×10⁵ to 2.0×10⁵ cells/kg, 1.5×10⁵ to 2.5×10⁵ cells/kg, 2.0×10⁵ to 3.0×10⁵ cells/kg, 2.5×10⁵ to 3.5×10⁵ cells/kg, 3.0×10⁵ to 4.0×10⁵ cells/kg, 3.5×10⁵ to 4.5×10⁵ cells/kg, 4.0×10⁵ to 5.0×10⁵ cells/kg, 4.5×10⁵ to 5.5×10⁵ cells/kg, 5.0×10⁵ to 6.0×10⁵ cells/kg, 5.5×10⁵ to 6.5×10⁵ cells/kg, 6.0×10⁵ to 7.0×10⁵ cells/kg, 6.5×10⁵ to 7.5×10⁵ cells/kg, 7.0×10⁵ to 8.0×10⁵ cells/kg, 7.5×10⁵ to 8.5×10⁵ cells/kg, 8.0×10⁵ to 9.0×10⁵ cells/kg, 8.5×10⁵ to 9.5×10⁵ cells/kg, 9.0×10⁵ to 1.0×10⁶ cells/kg. In a preferred embodiment, the dose is formulated at approximately 0.75×10⁶ cells/kg. In certain embodiments, the CAR-T cells are formulated at a dose of less than 1.0×10⁸ cells per subject.

### 4.7. Methods of Treating Subjects

The present application further relates to methods and compositions for use in cell immunotherapy. In some embodiments, the cell immunotherapy is for treating cancer in a subject, including but not limited to hematological malignancies and solid tumors. In some embodiments, the cell immunotherapy is for treating multiple myeloma in a subject. In some embodiments, the subject is human. In some embodiments, the methods are suitable for treatment of adults and pediatric population, including all subsets of age, and can be used as any line of treatment, including first line or subsequent lines.

Any of the anti-BCMA VHHs, CARs, and engineered immune effector cells (such as CAR-T cells) described herein may be used in the method of treating cancer. In some embodiments, the immune effector cells are autologous. In some embodiments, the immune effector cells are allogeneic.

In certain embodiments, the CAR-T cells are administered at a dose of about 1.0×10⁵ to 2.0×10⁵ cells/kg, 1.5×10⁵ to 2.5×10⁵ cells/kg, 2.0×10⁵ to 3.0×10⁵ cells/kg, 2.5×10⁵ to 3.5×10⁵ cells/kg, 3.0×10⁵ to 4.0×10⁵ cells/kg, 3.5×10⁵ to 4.5×10⁵ cells/kg, 4.0×10⁵ to 5.0×10⁵ cells/kg, 4.5×10⁵ to 5.5×10⁵ cells/kg, 5.0×10⁵ to 6.0×10⁵ cells/kg, 5.5×10⁵ to 6.5×10⁵ cells/kg, 6.0×10⁵ to 7.0×10⁵ cells/kg, 6.5×10⁵ to 7.5×10⁵ cells/kg, 7.0×10⁵ to 8.0×10⁵ cells/kg, 7.5×10⁵ to 8.5×10⁵ cells/kg, 8.0×10⁵ to 9.0×10⁵ cells/kg, 8.5×10⁵ to 9.5×10⁵ cells/kg, 9.0×10⁵ to 1.0×10⁶ cells/kg, 1.0×10⁶ to 2.0×10⁶ cells/kg, 1.5×10⁶ to 2.5×10⁶ cells/kg, 2.0×10⁶ to 3.0×10⁶ cells/kg, 2.5×10⁶ to 3.5×10⁶ cells/kg, 3.0×10⁶ to 4.0×10⁶ cells/kg, 3.5×10⁶ to 4.5×10⁶ cells/kg, 4.0×10⁶ to 5.0×10⁶ cells/kg, 4.5×10⁶ to 5.5×10⁶ cells/kg, or 5.0×10⁶ to 6.0×10⁶ cells/kg. In certain embodiments, the dose comprises approximately 0.75×10⁶ cells/kg. In certain embodiments, the dose comprises approximately 0.68×10⁶ cells/kg. In certain embodiments, the CAR-T cells are administered at a dose of about 1.0×10⁸ cells per subject.

In certain embodiments, the CAR-T cells are administered at a dose of less than 1.0×10⁸ cells per subject. In certain embodiments, the CAR-T cells are administered at a dose of about 3.0 to 4.0×10⁷ cells. In certain embodiments, the CAR-T cells are administered at a dose of about 3.5 to 4.5×10⁷ cells. In certain embodiments, the CAR-T cells are administered at a dose of about 4.0 to 5.0×10⁷ cells. In certain embodiments, the CAR-T cells are administered at a dose of about 4.5 to 5.5×10⁷ cells. In certain embodiments, the CAR-T cells are administered at a dose of about 5.0 to 6.0×10⁷ cells. In certain embodiments, the CAR-T cells are administered at a dose of about 5.5 to 6.5×10⁷ cells. In certain embodiments, the CAR-T cells are administered at a dose of about 6.0 to 7.0×10⁷ cells. In certain embodiments, the CAR-T cells are administered at a dose of about 6.5 to 7.5×10⁷ cells. In certain embodiments, the CAR-T cells are administered at a dose of about 7.0 to 8.0×10⁷ cells. In certain embodiments, the CAR-T cells are administered at a dose of about 7.5 to 8.5×10⁷ cells. In certain embodiments, the CAR-T cells are administered at a dose of about 8.0 to 9.0×10⁷ cells. In certain embodiments, the CAR-T cells are administered at a dose of about 8.5 to 9.5×10⁷ cells. In certain embodiments, the CAR-T cells are administered at a dose of about 9.0×10⁷ to 1.0×10⁸ cells.

In certain embodiments, the CAR-T cells are administered at a dose of about 0.693×10⁶ CAR-positive viable T-cells/kg. In certain embodiments, the CAR-T cells are administered at a dose of about 0.52×10⁶ CAR-positive viable T-cells/kg. In certain embodiments, the CAR-T cells are administered at a dose of about 0.94×10⁶ CAR-positive viable T-cells/kg. In certain embodiments, the CAR-T cells are administered at a dose of about 0.709×10⁶ CAR-positive viable T-cells/kg. In certain embodiments, the CAR-T cells are administered at a dose of about 0.51×10⁶ CAR-positive viable T-cells/kg. In certain embodiments, the CAR-T cells are administered at a dose of about 0.95×10⁶ CAR-positive viable T-cells/kg. In certain embodiments, the CAR-T cells are administered in an outpatient setting.

In certain embodiments, the CAR-T cells (e.g., at any of the foregoing doses) are administered in one or more intravenous infusions. In certain embodiments, said administration of said CAR-T cells is via a single intravenous infusion. In certain embodiments, said single intravenous infusion is administered using a single bag of said CAR-T cells. In certain embodiments, said administration of said single bag of said CAR-T cells is completed between the time at which said single bag of CAR-T cells is thawed and three hours after said single bag of CAR-T cells is thawed. In certain embodiments, single intravenous administration is administered using two bags of said CAR-T cells. In certain embodiments, said administration of each of said two bags of said CAR-T cells is completed between the time at which a first bag of said two bags of CAR-T cells is thawed and three hours after said first bag of CAR-T cells is thawed.

In certain embodiments, the time since the initial apheresis to the administration of CAR-T cells is less than 41, 47, 54, 61, 68, 75, 82, 89, 96, 103, 110, 117, 124, 131, 138, 145, 152, 159, 166 or 167 days. In certain embodiments, the time since the initial apheresis to the administration of CAR-T cells is greater than 41, 47, 54, 61, 68, 75, 82, 89, 96, 103, 110, 117, 124, 131, 138, 145, 152, 159, 166 or 167 days.

The composition comprising the host cells expressing the CAR-encoding nucleic acid sequence disclosed herein, or a vector comprising the CAR-encoding nucleic acid sequence disclosed herein, can be administered to a mammal using standard administration techniques, including oral, intravenous, intraperitoneal, subcutaneous, pulmonary, transdermal, intramuscular, intranasal, buccal, sublingual, or suppository administration. The composition preferably is suitable for parenteral administration. The term “parenteral”, as used herein, includes intravenous, intramuscular, subcutaneous, rectal, vaginal, and intraperitoneal administration. More preferably, the composition is administered to a mammal using peripheral systemic delivery by intravenous, intraperitoneal, or subcutaneous injection. Most preferably, the composition is administered by intravenous infusion.

The composition comprising the host cells expressing the CAR-encoding nucleic acid sequence disclosed herein, or a vector comprising the CAR-encoding nucleic acid sequence disclosed herein, can be administered with one or more additional therapeutic agents, which can be coadministered to the mammal. By “coadministering” is meant administering one or more additional therapeutic agents and the composition comprising the host cells disclosed herein or the vector disclosed herein sufficiently close in time such that the CAR disclosed herein can enhance the effect of one or more additional therapeutic agents, or vice versa. In this regard, the composition comprising the host cells disclosed herein or the vector disclosed herein can be administered first, and the one or more additional therapeutic agents can be administered second, or vice versa.

A CAR-expressing cell described herein and the at least one additional therapeutic agent can be administered simultaneously, in the same or in separate compositions, or sequentially. For sequential administration, the CAR-expressing cell described herein can be administered first, and the additional agent can be administered second, or the order of administration can be reversed.

In certain embodiments, a lymphodepleting regimen precedes said administration of CAR-T cells by approximately 5 days to approximately 7 days. In certain embodiments, lymphodepleting regimen is administered intravenously. In certain embodiments, said lymphodepleting regimen comprises administration of cyclophosphamide or administration of fludarabine. In certain embodiments, said cyclophosphamide is administered intravenously at 300 mg/m². In certain embodiments, said fludarabine is administered intravenously at 30 mg/m². In certain embodiments, a lymphodepleting regimen comprising cyclophosphamide administered intravenously at 300 mg/m² and fludarabine administered intravenously at 30 mg/m² precedes said administration of CAR-T cells by approximately 5 days to approximately 7 days.

In certain embodiments, the subject further receives bridging therapy, wherein said bridging therapy comprises short-term treatment with at least one bridging medicament between apheresis and said lymphodepleting regimen. In certain embodiments, said at least one bridging medicament had previously obtained an outcome of stable disease, minimal response, partial response, very good partial response, complete response or stringent complete response for the subject. In certain embodiments, the subject had an increase in tumor burden despite said bridging therapy. In certain embodiments, the subject had an increase in tumor burden of approximately 25% or greater despite said bridging therapy.

In certain embodiments, the subject is treated with pre-administration medication comprising an antipyretic and an antihistamine up to approximately 1 hour before said administration of said CAR-T cells. In certain embodiments, said antipyretic comprises either paracetamol or acetaminophen. In certain embodiments, said antipyretic is administered to the subject either orally or intravenously. In certain embodiments, said antipyretic is administered to the subject at a dosage of between 650 mg and 1000 mg. In certain embodiments, said antihistamine comprises diphenhydramine. In certain embodiments, said antihistamine is administered to the subject either orally or intravenously. In certain embodiments, said antihistamine is administered at a dosage of between 25 mg and 50 mg, or its equivalent. In certain embodiments, said antipyretic comprises either paracetamol or acetaminophen and said antipyretic is administered to the subject either orally or intravenously at a dosage of between 650 mg and 1000 mg, and wherein said antihistamine comprises diphenhydramine and said antihistamine is administered to the subject either orally or intravenously at a dosage of between 25 mg and 50 mg, or its equivalent.

In some embodiments, the method further comprises diagnosing said subject for cytokine release syndrome (CRS). In preferred embodiments, the diagnosis is made according to the American Society of Transplantation and Cellular Therapy (ASTCT), formerly the American Society for Blood and Marrow Transplantation (ASBMT) consensus grading. A non-limiting summary of the ASTCT consensus grading for CRS diagnosis is provided in Table 34. In some embodiments, the CRS is assessed by evaluating the levels of one or more of, or all of, IL-6, IL-10, IFN-γ, C-reactive protein (CRP) and ferritin.

In some embodiments, the method further comprises treating said subject for cytokine release syndrome (CRS). In some embodiments, the treatment of CRS is with an antipyretic. In some examples, the treatment of CRS is with anticytokine therapy. In some embodiments, the treatment of CRS occurs more than approximately 3 days following the infusion. In some embodiments, the treatment of CRS occurs without significantly reducing CAR-T cell expansion in vivo. In certain embodiments, said method further comprises treating said subject for cytokine release syndrome more than approximately 3 days following said administration of said CAR-T cells without significantly reducing expansion of said CAR-T cells in vivo. In some embodiments, the treatment of CRS comprises administering to the subject an IL-6R inhibitor. In some embodiments, the IL-6R inhibitor is an antibody. In some embodiments, the antibody inhibits IL-6R by binding its extracellular domain. In some embodiments, the IL-6R inhibitor prevents the binding of IL-6 to IL-6R. In some embodiments, the IL-6R inhibitor is tocilizumab. In some embodiments, the anticytokine therapy comprises administration of tocilizumab. In some embodiments, the anticytokine therapy comprises administration of steroids. In some embodiments, treatment for CRS comprises treatment with monoclonal antibodies other than tocilizumab. In some embodiments, the antibodies other than tocilizumab target cytokines. In some embodiments, the cytokine that the antibodies other than tocilizumab target is IL-1. In some embodiments, the IL-1 targeting antibody is Anakinra. In some embodiments, the cytokine that the antibodies other than tocilizumab target is TNFα. In some embodiments, the treatment of CRS comprises administering to the subject a corticosteroid. In some embodiments, the treatment of CRS comprises using a vasopressor. In some embodiments, the treatment of CRS comprises intubation or mechanical ventilation. In some embodiments, the treatment of CRS comprises administering to the subject cyclophosphamide. In some embodiments, the treatment of CRS comprises administering to the subject etanercept. In some embodiments, the treatment of CRS comprises administering to the subject levetiracetam. In some embodiments, the treatment of CRS comprises supportive care.

In some embodiments, the method further comprises diagnosing said subject for immune cell effector-associated neurotoxicity (ICANS). In some embodiments, the diagnosis is made according to the National Cancer Institute Common Terminology Criteria for Adverse Events (NCI CTCAE) criteria. In some embodiments, the diagnosis is made according to the NCI CTCAE criteria, Version 5.0. In some embodiments, the diagnosis is made according to the American Society of Transplantation and Cellular Therapy (ASTCT) consensus grading system. In some embodiments, the embodiments, there is neurotoxicity consistent with ICAN. A non-limiting summary of the ASTCT consensus grading system for ICANS diagnosis is provided in Table 35. In some embodiments, the treatment of ICANS comprises administering to the subject an IL-6R inhibitor. In some embodiments, the IL-6R inhibitor is an antibody. In some embodiments, the antibody inhibits IL-6R by binding its extracellular domain. In some embodiments, the IL-6R inhibitor prevents the binding of IL-6 to IL-6R. In some embodiments, the IL-6R inhibitor is tocilizumab. In some embodiments, the treatment of ICANS comprises administering to the subject an IL-1 inhibitor. In some embodiments the IL-1 inhibitor is an antibody. In a preferred embodiment, the IL-1 inhibiting antibody is Anakinra. In some embodiments, the treatment of ICANS comprises administering to the subject a corticosteroid. In some embodiments, the treatment of ICANS comprises administering to the subject levetiracetam. In some embodiments, the treatment of ICANS comprises administering to the subject dexamethasone. In some embodiments, the treatment of ICANS comprises administering to the subject methylprednisone sodium succinate. In some embodiments, the treatment of ICANS comprises administering to the subject pethidine. In some embodiments, the treatment of ICANS comprises administering to the subject one or more of, or all of, tocilizumab, Anakinra, a corticosteroid, levetiracetam, dexamethasone, methylprednisone sodium succinate or pethidine.

In some embodiments, the method further comprises diagnosing said subject for cytopenias. In some embodiments, the cytopenias comprise one or more of, or all of, lymphopenia, neutropenia, and thrombocytopenia. Without being bound by theory, a Grade 3 or Grade 4 but not a Grade 2 or lower lymphopenia is characterized by to a lymphocyte count less than 0.5×10⁹ cells per liter of a subject's blood sample, a Grade 3 or Grade 4 but not a Grade 2 or lower neutropenia is characterized by a neutrophil count less than 1000 cells per microliter of a subject's blood sample, and a Grade 3 or Grade 4 but not a Grade 2 or lower thrombocytopenia is characterized by a platelet count less than 50,000 cells per microliter of a subject's blood sample. In some embodiments, greater than 75% subjects with Grade 3 or Grade 4 lymphopenia following CAR-T cell administration recover to Grade 2 or lower lymphopenia 60 days following CAR-T cell administration. In some embodiments, greater than 80% subjects with Grade 3 or Grade 4 lymphopenia following CAR-T cell administration recover to Grade 2 or lower lymphopenia 60 days following CAR-T cell administration. In some embodiments, greater than 85% subjects with Grade 3 or Grade 4 lymphopenia following CAR-T cell administration recover to Grade 2 or lower lymphopenia 60 days following CAR-T cell administration. In some embodiments, greater than 90% subjects with Grade 3 or Grade 4 lymphopenia following CAR-T cell administration recover to Grade 2 or lower lymphopenia 60 days following CAR-T cell administration. In some embodiments, greater than 70% subjects with Grade 3 or Grade 4 neutropenia following CAR-T cell administration recover to Grade 2 or lower neutropenia 60 days following CAR-T cell administration. In some embodiments, greater than 75% subjects with Grade 3 or Grade 4 neutropenia following CAR-T cell administration recover to Grade 2 or lower neutropenia 60 days following CAR-T cell administration. In some embodiments, greater than 80% subjects with Grade 3 or Grade 4 neutropenia following CAR-T cell administration recover to Grade 2 or lower neutropenia 60 days following CAR-T cell administration. In some embodiments, greater than 85% subjects with Grade 3 or Grade 4 neutropenia following CAR-T cell administration recover to Grade 2 or lower neutropenia 60 days following CAR-T cell administration. In some embodiments, greater than 30% subjects with Grade 3 or Grade 4 thrombocytopenia following CAR-T cell administration recover to Grade 2 or lower thrombocytopenia 60 days following CAR-T cell administration. In some embodiments, greater than 34% subjects with Grade 3 or Grade 4 thrombocytopenia following CAR-T cell administration recover to Grade 2 or lower thrombocytopenia 60 days following CAR-T cell administration. In some embodiments, greater than 38% subjects with Grade 3 or Grade 4 thrombocytopenia following CAR-T cell administration recover to Grade 2 or lower thrombocytopenia 60 days following CAR-T cell administration. In some embodiments, greater than 42% subjects with Grade 3 or Grade 4 thrombocytopenia following CAR-T cell administration recover to Grade 2 or lower thrombocytopenia 60 days following CAR-T cell administration.

Once the composition comprising host cells expressing the CAR-encoding nucleic acid sequence disclosed herein, or a vector comprising the CAR-encoding nucleic acid sequence disclosed herein, is administered to a mammal (e.g., a human), the biological activity of the CAR can be measured by any suitable method known in the art. In accordance with the method disclosed herein, the CAR binds to BCMA on the multiple myeloma cells, and the multiple myeloma cells are destroyed. Binding of the CAR to BCMA on the surface of multiple myeloma cells can be assayed using any suitable method known in the art, including, for example, ELISA and flow cytometry. The ability of the CAR to destroy multiple myeloma cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). The biological activity of the CAR also can be measured by assaying expression of certain cytokines, such as CD 107a, IFNγ, IL-2, and TNF.

The methods described herein may be used for treating various cancers, including both solid cancer and liquid cancer. In certain embodiments, the methods are used to treat multiple myeloma. The methods described herein may be used as a combination therapy with other types of cancer therapies known in the art, such as chemotherapy, surgery, radiation, gene therapy, immunotherapy, bone marrow transplantation, stem cell transplantation, targeted therapy, cryotherapy, ultrasound therapy, photodynamic therapy, radio-frequency ablation or the like, in an adjuvant setting or a neoadjuvant setting.

In certain embodiments, the cancer is multiple myeloma. In certain embodiments, the cancer is stage I, stage II or stage III, and/or stage A or stage B multiple myeloma based on the Durie-Salmon staging system. In certain embodiments, the cancer is stage I, stage II or stage III multiple myeloma based on the International staging system published by the International Myeloma Working Group (IMWG). In some embodiments, the multiple myeloma is progressive.

In certain embodiments, the subject received prior treatment with at least an initial therapy. In certain embodiments, the initial therapy comprises treatment with a medicament that is a proteasomal inhibitor (PI). Non-limiting examples of a PI include bortezomib, carfilzomib and ixazomib. In certain embodiments, the initial therapy comprises treatment with a medicament that is an immunomodulatory drug (IMiD). Non-limiting examples of an IMiD include lenalidomide, pomalidomide, thalidomide, and cereblon E3 ligase modulatory drugs (CELMoD), such as iberdomine or mezigdomide. In certain embodiments, the initial therapy comprises treatment with a medicament that is a corticosteroid. Non-limiting examples of a corticosteroid include dexamethasone and prednisone. In certain embodiments, the initial therapy comprises treatment with a medicament that is an alkylating agent. In certain embodiments, the initial therapy comprises treatment with a medicament that is an anthracycline. In certain embodiments, the initial therapy comprises treatment with a medicament that is an anti-CD38 antibody. Non-limiting examples of an anti-CD38 antibody include daratumumab, isatuximab and the investigational antibody TAK-079. In certain embodiments, the initial therapy comprises treatment with a medicament that is elotuzumab. In certain embodiments, the initial therapy comprises treatment with a medicament that is panobinostat. In certain embodiments, the cancer is refractory to one or more of, or all of, bortezomib, carfilzomib, ixazomib, lenalidomide, pomalidomide, thalidomide, dexamethasone, prednisone, alkylating agents, daratumumab, isatuximab, TAK-079, elotuzumab, Panobinostat, or a cereblon E3 ligase modulatory drug (CELMoD), such as iberdomine or mezigdomide. In certain embodiments, the initial therapy includes surgery, radiotherapy, or autologous or allogeneic transplant, including tandem transplants, or any combination of such treatments.

In some embodiments, the multiple myeloma is refractory to at least two medicaments. In some embodiments, the multiple myeloma is refractory to at least three medicaments. In some embodiments, the multiple myeloma is refractory to at least four medicaments. In some embodiments, the multiple myeloma is refractory to at least five medicaments.

In some embodiments, the subject has bone marrow plasma cells of between approximately 10% and approximately 30% before said administration of said CAR-T cells.

In certain embodiments, bone marrow aspirate or biopsy may be performed for clinical assessments or bone marrow aspirate may be performed for biomarker evaluations. In certain embodiments, clinical staging (morphology, cytogenetics, and immunohistochemistry or immunofluorescence or flow cytometry) may be done. In certain embodiments, a portion of the bone marrow aspirate may be immunophenotyped and monitored for BCMA, checkpoint ligand expression in CD138-positive multiple myeloma cells, and checkpoint expression on T cells. In certain embodiments, minimal residual disease (MRD) may be monitored in subjects using next generation sequencing (NGS) of bone marrow aspirate DNA. The NGS of bone marrow aspirate DNA is known to one of ordinary skill in the art. In certain embodiments, the NGS is performed via clonoSEQ. In certain embodiments, baseline bone marrow aspirates may be used to define the myeloma clones, and post-treatment samples may be used to evaluate MRD negativity. In certain embodiments, the MRD negativity status may be based on samples that are evaluable. In certain embodiments, evaluable samples are those that passed one or more of, or all of, calibration, quality control, and sufficiency of cells evaluable at a particular sensitivity level. In some embodiments, the sensitivity level is 10⁻⁶. In certain embodiments, the sensitivity level is 10⁻⁶, the sensitivity level is 10⁻⁵. In certain embodiments, the sensitivity level is 10⁻⁴. In certain embodiments, the sensitivity level is 10⁻³.

In certain embodiments, a subject's response to the method of treatment is assessed using the International Myeloma Working Group (IMWG)-based response criteria, which are summarized in Table 33. In certain embodiments, the response may be classified as a stringent complete response (sCR). In certain embodiments, the response may be classified as a complete response (CR), which is worse than a stringent complete response (sCR). In certain embodiments, the response may be classified as a very good partial response (VGPR), which is worse than a complete response (CR). In certain embodiments, the response may be classified as a partial response (PR), which is worse than a very good partial response (VGPR). In certain embodiments, the response may be classified as a minimal response (MR), which is worse than a partial response (PR). In certain embodiments, the response may be classified as a stable disease (SD), which is worse than a minimal response (MR). In certain embodiments, the response may be classified as a progressive disease (PD), which is worse than a stable disease.

In some embodiments, the method achieves an overall response rate of about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of treated subjects. The overall response rate may be deciphered by calculating the proportion of patients who achieve a partial response, a very good partial response, a complete response, or a stringent complete response.

In certain embodiments, the tests used to assess International Myeloma Working Group (IMWG)-based response criteria are Myeloma protein (M-protein) measurements in serum and urine, serum calcium corrected for albumin, bone marrow examination, skeletal survey and documentation of extramedullary plasmacytomas.

Non-limiting examples of tests for M-protein measurement in blood and urine are known to one of ordinary skill in the art and comprise serum quantitative Ig, serum protein electrophoresis (SPEP), serum immunofixation electrophoresis, serum FLC assay, 24-hour urine M-protein quantitation by electrophoresis (UPEP), urine immunofixation electrophoresis, and serum β2-microglobulin.

Calculating serum calcium corrected for albumin in blood samples for detection of hypercalcemia is known to one of ordinary skill in the art. Without wishing to be bound by theory, calcium binds to albumin and only the unbound (free) calcium is biologically active; therefore, the serum calcium level must be adjusted for abnormal albumin levels (“corrected serum calcium”).

In certain embodiments, a skeletal survey of any one of, or all of, the skull, the entire vertebral column, the pelvis, the chest, the humeri, the femora, and any other bones, may be performed and evaluated by either roentgenography (“X-rays”) or low-dose computed tomography (CT) diagnostic quality scans without the use of IV contras, both of which are known to one of ordinary skill in the art. In certain embodiments, following T cell administration and before disease progression is confirmed, X-rays or CT scans may be performed locally, whenever clinically indicated based on symptoms, to document response or progression. In certain embodiments, magnetic resonance imaging (MRI) may be used for evaluating bone disease but does not replace a skeletal survey. MRI is known to one of ordinary skill in the art. In certain embodiments, if a radionuclide bone scan is used at screening, in addition to the complete skeletal survey, both methods may be used to document disease status. Radionuclide bone scans are known to one of ordinary skill in the art. In certain embodiments, the radionuclide bone scan and complete skeletal survey may be performed at the same time. In certain embodiments, a radionuclide bone scan may not replace a complete skeletal survey. In certain embodiments, if a subject presents with disease progression manifested by symptoms of pain due to bone changes, then disease progression may be documented by skeletal survey or other radiographs, depending on the symptoms that the subject experiences.

In certain embodiments, extramedullary plasmacytomas may be documented by clinical examination or MRI. In certain embodiments, if there was no contraindication to the use of IV contrast, extramedullary plasmacytomas may be documented by CT scan. In certain embodiments, extramedullary plasmacytomas may be documented by a fusion of positron emission tomography (PET) and CT scans if the CT component is of sufficient diagnostic quality. In certain embodiments, assessment of measurable sites of extramedullary disease may be performed, measured, or evaluated locally every 4 weeks for subjects until development of confirmed CR or confirmed disease progression. In certain embodiments, evaluation of extramedullary plasmacytomas may be done every 12 weeks.

In certain embodiments, to qualify for VGPR or PR or MR, the sum of products of the perpendicular diameters of the existing extramedullary plasmacytomas may have decreased by over 90% or at least 50%, respectively. In certain embodiments, to qualify for disease progression, either the sum of products of the perpendicular diameters of the existing extramedullary plasmacytomas must have increased by at least 50%, or the longest diameter of previous lesion >1 cm in short axis must have increased at least 50%, or a new plasmacytoma must have developed. In certain embodiments, to qualify for disease progression when not all existing extramedullary plasmacytomas are reported, the sum of products of the perpendicular diameters of the reported plasmacytomas had increased by at least 50%. In certain embodiments, if the study treatment interferes with the immunofixation assay, CR may be defined as the disappearance of the original M-protein associated with multiple myeloma on immunofixation.

In certain embodiments, a subject's response to the method of treatment is assessed in terms of change in disease burden or tumor burden. Disease burden or tumor burden represents the type of measurable disease in the subject. In some embodiments, the change in tumor burden may be assessed in terms of paraprotein level changes upon treatment. In some embodiments, the paraprotein is an M-protein in the serum. In some embodiments, the paraprotein is an M-protein in the serum. In some embodiments, the change in tumor burden is assessed in terms of the difference between involved and uninvolved free light chain (dFLC). In some embodiments, the change in tumor burden is assessed in terms of the maximum paraprotein reduction from baseline, i.e., from prior to the administration of the CAR-T cells. In some embodiments, the change in tumor burden is assessed at a median follow-up time of greater than or equal to 28 days following the administration of CAR-T cells. In some embodiments, the change in tumor burden is assessed at a median follow-up time of greater than or equal to 1 month following the administration of CAR-T cells. In some embodiments, the change in tumor burden is assessed at a median follow-up time of greater than or equal to 3 months following the administration of CAR-T cells. In some embodiments, the change in tumor burden is assessed at a median follow-up time of greater than or equal to 6 months following the administration of CAR-T cells. In some embodiments, the change in tumor burden is assessed at a median follow-up time of greater than or equal to 9 months following the administration of CAR-T cells. In some embodiments, the change in tumor burden is assessed at a median follow-up time of greater than or equal to 12 months following the administration of CAR-T cells.

In certain embodiments, the subject is re-treated by administration via a second intravenous infusion of a second dose of CAR-T cells. In certain embodiments, the re-treatment dose comprises 1.0×10⁵ to 5.0×10⁶ of CAR-T cells per kilogram of the mass of the subject. In certain embodiments, the re-treatment dose comprises approximately 0.75×10⁵ of CAR-T cells per kilogram of the mass of the subject. In certain embodiments, the subject is re-treated upon exhibiting progressive disease after a best response of minimal response or better following the first infusion of CAR-T cells. In certain embodiments, the time between the first infusion of CAR-T cells and the detection of the progressive disease comprises at least six months.

4.8. Methods of Treating Subjects with Prior Stem Cell Transplantation

In one aspect is provided a method of treating a subject with multiple myeloma wherein the subject has not achieved a complete response after receiving an initial therapy comprising a stem cell transplantation, the method comprising administering to the subject a dose of T cells comprising a chimeric antigen receptor (CAR) provided herein.

In one aspect is provided a method of treating a subject with multiple myeloma wherein the subject has not achieved a complete response after receiving an initial therapy comprising a stem cell transplantation, the method comprising administering to the subject a dose of T cells comprising a chimeric antigen receptor (CAR) comprising:
(a) an extracellular antigen binding domain capable of specifically binding to an epitope of B-cell maturation antigen (BCMA), wherein the extracellular antigen binding domain comprises a first VHH domain and a second VHH domain, and wherein the first VHH domain comprising a CDR1, a CDR2, and a CDR3 as set forth in the VHH domain comprising the amino acid sequence of SEQ ID NO: 2, and the second VHH domain comprising a CDR1, a CDR2, and a CDR3 as set forth in the VHH domain comprising the amino acid sequence of SEQ ID NO: 4,
(b) a transmembrane domain, and
(c) an intracellular signaling domain.

In some embodiments, the initial therapy further comprises (1) 4-8 cycles of an induction therapy and (2) a high-dose chemotherapy.

In some embodiments, the stem cell transplantation is autologous or allogeneic stem cell transplantation. In some embodiments, the stem cell transplantation is autologous stem cell transplantation (ASCT). In some embodiments, the stem cell transplantation is allogeneic stem cell transplantation. In some embodiments, the stem cell transplantation is a tandem stem cell transplantation. In some embodiments, the tandem stem cell transplantation is tandem ASCT.

In some embodiments, the high-dose chemotherapy comprises melphalan.

The term “induction” or “induction therapy” refers to treatment regimens in the initial therapy to achieve adequate disease control, allow adequate stem-cell harvest, induce the deepest possible response, and minimize toxicity (Bazarbachi et al., Blood Cancer Journal, 12(47): 1-8 (2022)). The updated 2021 European Haematology Association (EHA) and European Society for Medical Oncology (EMSO) guidelines developed from the 2007 EMSO proposal of a systemic triplet induction comprising PI bortezomib plus dexamethasone (Vd) backbone with the addition of a third agent such as thalidomide (VTd), cyclophosphamide (VCd), lenalidomide (VRd) or doxorubicin (PAd) to incorporate more novel agents such as VRd or VTd plus the CD38 monoclonal antibody (mAb) daratumumab (Dara-VTd) as first options, when unavailable, VTd or VCd as second options (Bazarbachi et al., Blood Cancer Journal, 12(47): 1-8 (2022)). In some embodiments, the induction therapy comprises a proteasomal inhibitor (PI) or an immunomodulatory drug (IMiD). In some embodiments, the induction therapy comprises a proteasomal inhibitor (PI) and an immunomodulatory drug (IMiD). In some embodiments, the PI is bortezomib, carfilzomib, or ixazomib. In some embodiments, the IMiD is lenalidomide, pomalidomide, thalidomide, or a cereblon E3 ligase modulatory drug (CELMoD), such as iberdomine or mezigdomide. In some embodiments, the induction therapy further comprises an alkylating agent. In some embodiments, the alkylating agent is cyclophosphamide. In some embodiments, the induction therapy further comprises an anti-CD38 antibody. In some embodiments, the anti-CD38 antibody is daratumumab.

In some embodiments, the method further comprises administering to the subject a dose of an immunomodulatory drug (IMiD) after administering to the subject the dose of the T cells.

In some embodiments, the subject is not refractory to the IMiD administered after the dose of T cells. In some embodiments, the IMiD is lenalidomide, pomalidomide, thalidomide, or a cereblon E3 ligase modulatory drug (CELMoD), such as iberdomine or mezigdomide. In some embodiments, the IMiD is lenalidomide. In some embodiments, the dose of lenalidomide is about 2.5 mg, about 5 mg, about 10 mg, about 15 mg, about 20 mg, about 25 mg, about 30 mg, about 35 mg, about 40 mg, about 45 mg, or about 50 mg daily. In some embodiments, the dose of lenalidomide is about 2.5 mg, about 5 mg, about 10 mg, or about 15 mg daily. In some embodiments, the dose of lenalidomide is about 2.5 mg daily. In some embodiments, the dose of lenalidomide is about 5 mg daily. In some embodiments, the dose of lenalidomide is about 10 mg daily. In some embodiments, the dose of lenalidomide is about 15 mg daily. In some embodiment, the dose of lenalidomide is about 20 mg daily. In some embodiments, the dose of lenalidomide is about 25 mg daily. In some embodiments, the dose of lenalidomide is about 30 mg daily. In some embodiments, the dose of lenalidomide is about 35 mg daily. In some embodiment, the dose of lenalidomide is about 40 mg daily. In some embodiments, the dose of lenalidomide is about 45 mg daily. In some embodiments, the dose of lenalidomide is about 50 mg daily.

In some embodiments, the administering of the dose of lenalidomide is once daily. In some embodiments, the administering of the dose of lenalidomide is 2 or 3 times daily.

In some embodiments, the administering of the dose of lenalidomide starts between about 21 days to about 214 days after the administering of the dose of the T cells to the subject. In some embodiments, the administering of the dose of lenalidomide starts between about 10 days to about 240 days after the administering of the dose of the T cells to the subject. In some embodiments, the administering of the dose of lenalidomide starts between about 15 days to about 230 days after the administering of the dose of the T cells to the subject. In some embodiments, the administering of the dose of lenalidomide starts between about 20 days to about 220 days after the administering of the dose of the T cells to the subject. In some embodiments, the administering of the dose of lenalidomide starts between about 25 days to about 210 days after the administering of the dose of the T cells to the subject.

In some embodiments, the administering of the dose of lenalidomide starts at a median of about 51 days or at a mean of about 85.1 days. In some embodiments, the administering of the dose of lenalidomide starts at a median or a mean of about 20 days. In some embodiments, the administering of the dose of lenalidomide starts at a median or a mean of about 25 days. In some embodiments, the administering of the dose of lenalidomide starts at a median or a mean of about 30 days. In some embodiments, the administering of the dose of lenalidomide starts at a median or a mean of about 35 days. In some embodiments, the administering of the dose of lenalidomide starts at a median or a mean of about 40 days. In some embodiments, the administering of the dose of lenalidomide starts at a median or a mean of about 45 days. In some embodiments, the administering of the dose of lenalidomide starts at a median or a mean of about 50 days. In some embodiments, the administering of the dose of lenalidomide starts at a median or a mean of about 55 days. In some embodiments, the administering of the dose of lenalidomide starts at a median or a mean of about 60 days. In some embodiments, the administering of the dose of lenalidomide starts at a median or a mean of about 65 days. In some embodiments, the administering of the dose of lenalidomide starts at a median or a mean of about 70 days. In some embodiments, the administering of the dose of lenalidomide starts at a median or a mean of about 75 days. In some embodiments, the administering of the dose of lenalidomide starts at a median or a mean of about 80 days. In some embodiments, the administering of the dose of lenalidomide starts at a median or a mean of about 85 days. In some embodiments, the administering of the dose of lenalidomide starts at a median or a mean of about 90 days. In some embodiments, the administering of the dose of lenalidomide starts at a median or a mean of about 95 days.

In some embodiments, the administering of the dose of lenalidomide continues for between about 70 days to about 716 days. In some embodiments, the administering of the dose of lenalidomide continues for between about 50 days to about 750 days. In some embodiments, the administering of the dose of lenalidomide continues for between about 55 days to about 740 days. In some embodiments, the administering of the dose of lenalidomide continues for between about 60 days to about 730 days. In some embodiments, the administering of the dose of lenalidomide continues for between about 65 days to about 720 days. In some embodiments, the administering of the dose of lenalidomide continues for between about 70 days to about 715 days.

In some embodiments, the administering of the dose of lenalidomide continues for a median of about 426.5 days or at a mean of about 426.0 days. In some embodiments, the administering of the dose of lenalidomide continues for a median or a mean of about 300 days. In some embodiments, the administering of the dose of lenalidomide continues for a median or a mean of about 350 days. In some embodiments, the administering of the dose of lenalidomide continues for a median or a mean of about 400 days. In some embodiments, the administering of the dose of lenalidomide continues for a median or a mean of about 450 days. In some embodiments, the administering of the dose of lenalidomide continues for a median or a mean of about 500 days. In some embodiments, the administering of the dose of lenalidomide continues for a median or a mean of about 550 days. In some embodiments, the administering of the dose of lenalidomide continues for a median or a mean of about 600 days.

In some embodiments, the administering of the dose of lenalidomide is daily in a cycle of about 28 days. In some embodiments, the administering of the dose of lenalidomide is daily in a cycle of about 14 days. In some embodiments, the administering of the dose of lenalidomide is daily in a cycle of about 18 days. In some embodiments, the administering of the dose of lenalidomide is daily in a cycle of about 21 days. In some embodiments, the administering of the dose of lenalidomide is daily in a cycle of about 25 days. In some embodiments, the administering of the dose of lenalidomide is daily in a cycle of about 32 days. In some embodiments, the administering of the dose of lenalidomide is daily in a cycle of about 35 days. In some embodiments, the administering of the dose of lenalidomide is daily in a cycle of about 39 days. In some embodiments, the administering of the dose of lenalidomide is daily in a cycle of about 42 days.

In some embodiments, the administering of the dose of lenalidomide continues for between about 3 cycles to about 26 cycles. In some embodiments, the administering of the dose of lenalidomide continues for between about 1 cycle to about 30 cycles. In some embodiments, the administering of the dose of lenalidomide continues for between about 2 cycles to about 35 cycles. In some embodiments, the administering of the dose of lenalidomide continues for between about 3 cycles to about 40 cycles. In some embodiments, the administering of the dose of lenalidomide continues for between about 4 cycles to about 45 cycles. In some embodiments, the administering of the dose of lenalidomide continues for between about 5 cycles to about 50 cycles.

In some embodiments, the administering of the dose of the lenalidomide continues for a median or a mean of about 15 cycles. In some embodiments, the administering of the dose of the lenalidomide continues for a median or a mean of about 5, about 10, about 15, about 20, about 25, about 30, about 35, about 45, or about 50 cycles.

In some embodiments, the administering of the dose of the lenalidomide is at a relative dose intensity of between about 67.9% to about 100.0%. In some embodiments, the administering of the dose of the lenalidomide is at a relative dose intensity of between about 40% to about 100.0%. In some embodiments, the administering of the dose of the lenalidomide is at a relative dose intensity of between about 45.0% to about 100.0%. In some embodiments, the administering of the dose of the lenalidomide is at a relative dose intensity of between about 50.0% to about 100.0%. In some embodiments, the administering of the dose of the lenalidomide is at a relative dose intensity of between about 55.0% to about 100.0%. In some embodiments, the administering of the dose of the lenalidomide is at a relative dose intensity of between about 60.0% to about 100.0%. In some embodiments, the administering of the dose of the lenalidomide is at a relative dose intensity of between about 65.0% to about 100.0%. In some embodiments, the administering of the dose of the lenalidomide is at a relative dose intensity of between about 70.0% to about 100.0%. In some embodiments, the administering of the dose of the lenalidomide is at a relative dose intensity of between about 75.0% to about 100.0%. In some embodiments, the administering of the dose of the lenalidomide is at a relative dose intensity of between about 80.0% to about 100.0%. In some embodiments, the administering of the dose of the lenalidomide is at a relative dose intensity of between about 85.0% to about 100.0%. In some embodiments, the administering of the dose of the lenalidomide is at a relative dose intensity of between about 90.0% to about 100.0%. In some embodiments, the administering of the dose of the lenalidomide is at a relative dose intensity of between about 95.0% to about 100.0%.

In some embodiments, the administering of the dose of the lenalidomide is at a median relative dose intensity of about 93.4%. In some embodiments, the administering of the dose of the lenalidomide is at a median relative dose intensity of about 70.0%, about 75.0%, about 80.0%, about 85.0%, about 90.0%, about 92.0%, about 93.0%, about 94.0%, about 95.0%, about 96.0%, about 97.0%, about 98.0%, about 99.0%, or about 100.0%.

In some embodiments, the administering of the dose of lenalidomide continues until the later of when the subject is confirmed progressive disease or unacceptable toxicity, or when it has reached 2 years after start of the administering of the dose of lenalidomide.

In some embodiments, the subject has further or has not further received a consolidation therapy. The term “consolidation” or “consolidation therapy” refers to the therapy that is given to improve the response after a stem cell transplantation and to prevent early relapse (Sonneveld et al., Journal of Clinical Oncology, 39(32), 3613-22 (2021)). The same induction regimens as disclosed herein is usually used in the consolidation therapy (Bazarbachi et al., Blood Cancer Journal, 12(47): 1-8 (2022)). In some embodiments, the subject who has received a consolidation therapy has received 1, 2, 3, or 4 cycles of the consolidation therapy. In some embodiments, the subject who has received a consolidation therapy has received less than 2, 3, or 4 cycles of the consolidation therapy. In some embodiments, the subject who has received a consolidation therapy has received less than 2 cycles of the consolidation therapy. In some embodiments, the subject who has received a consolidation therapy has received less than 3 cycles of the consolidation therapy. In some embodiments, the subject who has received a consolidation therapy has received less than 4 cycles of the consolidation therapy.

In some embodiments, the subject has received an induction or a consolidation therapy comprising an immunomodulatory drug (IMiD) or a proteosomal inhibitor (PI), or a steroid, or any combination thereof. In some embodiments, the subject has received an induction or a consolidation therapy comprising an IMiD or a PI, or both, in combination with a steroid. In some embodiments, the subject has received an induction or a consolidation therapy comprising an IMiD in combination with a steroid. In some embodiments, the subject has received an induction or a consolidation therapy comprising a PI in combination with a steroid. In some embodiments, the subject has received an induction or a consolidation therapy comprising an IMiD and a PI in combination with a steroid.

In some embodiments, the subject who has not received a consolidation therapy has received an initial therapy comprising a stem cell transplantation for a least about 100 days. In some embodiments, the subject has not received a consolidation therapy has received the initial therapy comprising a stem cell transplantation for a least about 80, about 90 days, about 100 days, about 110 days, about 120 days, about 130 days, about 140 days, or about 150 days.

In some embodiments, the subject who has received a consolidation therapy has received an initial therapy comprising a stem cell transplantation for a least about 160 days. In some embodiments, the subject has not received a consolidation therapy has received an initial therapy comprising a stem cell transplantation for a least about 100, about 110 days, about 120 days, about 130 days, about 140 days, about 150 days, about 160 days, about 170 days, about 180 days, about 190 days, or about 200 days.

In some embodiments, the subject having multiple myeloma and treated as provided herein, has not yet evolved to progressive disease as assessed by IMWG criteria.

In some embodiments, the subject has further received a lymphodepletion therapy at least about 5 to about 7 days prior to the administering of the dose of the T cells. In some embodiments, the lymphodepletion therapy comprises administering cyclophosphamide and fludarabine daily. In some embodiments, the lymphodepletion therapy comprises cyclophosphamide at a concentration of about 300 mg/m² and fludarabine at a concentration of about 30 mg/m² daily for 3 days. In some embodiments, the lymphodepletion therapy was administered on day 7, day 6, and day 5 prior to the administering of the dose of the T cells.

In some embodiments, the dose of the T cells administered is about 0.5-1.0×10⁶ of the T cells/kg of body weight of the subject. In some embodiments, the dose of the T cells administered is about 0.56-0.84×10⁶ of the T cells/kg of body weight of the subject. In some embodiments, the dose of the T cells administered is at a mean of about 0.68×10⁶ of the T cells/kg of body weight of the subject. In some embodiments, the dose of the T cells administered is at a median of about 0.68×10⁶ of the T cells/kg of body weight of the subject. In some embodiments, the dose of the T cells administered is about 2.84-8.45×10⁷ of the T cells. In some embodiments, the dose of the T cells administered is at a mean of about 5.65×10⁷ of the T cells. In some embodiments, the dose of the T cells administered is at a median of about 5.43×10⁷ of the T cells. In some embodiments, the dose of the T cells is formulated to be about 0.60-0.80×10⁶ of the T cells/kg of body weight of the subject. In some embodiments, the dose of the T cells is formulated to be at a mean of about 0.67×10⁶ of the T cells/kg of body weight of the subject. In some embodiments, the dose of the T cells is formulated to be at a median of about 0.70×10⁶ of the T cells/kg of body weight of the subject.

In some embodiments, the administering of the dose of the T cells is in a single, two, or three infusions.

In some embodiments, the subject has further received a bridging therapy prior to the administering of the dose of the T cells. In some embodiments, the bridging therapy comprises a dose of lenalidomide. In some embodiments, the dose of lenalidomide is about 10 mg daily. In some embodiments, the dose of lenalidomide is about 5 mg daily. In some embodiments, the dose of lenalidomide starts at about 5 mg daily, and increases to about 10 mg daily. In some embodiments, the bridging therapy comprises one or more cycles of lenalidomide at a dose of about 10 mg daily. In some embodiments, a cycle of lenalidomide is about 28 days.

In certain embodiments, the method of treatment is effective in obtaining in the subject a reduction in tumor burden. In certain embodiments, the method of treatment is effective in obtaining in the subject a reduction in tumor burden of between approximately between approximately 1% and approximately 100%, between approximately 60% and approximately 100%, between approximately 65% and approximately 100%, between approximately 70% and approximately 100%, between approximately 75% and approximately 100%, between approximately 80% and approximately 100%, between approximately 85% and approximately 100%, between approximately 90% and approximately 100%, between approximately 92% and approximately 100%, between approximately 95% and approximately 100%, between approximately 96% and approximately 100%, between approximately 97% and approximately 100%, between approximately 98% and approximately 100%, or between approximately 99% and approximately 100%. In certain embodiments, the method of treatment is effective in obtaining in the subject a reduction in tumor burden of approximately 100%. In certain embodiments, the method of treatment is effective in obtaining in the subject a reduction in tumor burden of between approximately 1% and approximately 100% at a rate of between approximately 1% and approximately 100%. In certain embodiments, the method of treatment is effective in obtaining in the subject a reduction in tumor burden of between approximately 60% and approximately 100% at a rate of between approximately 1% and approximately 100%. In certain embodiments, the method of treatment is effective in obtaining in the subject a reduction in tumor burden of between approximately 65% and approximately 100% at a rate of between approximately 1% and approximately 92%. In certain embodiments, the method of treatment is effective in obtaining in the subject a reduction in tumor burden of between approximately 70% and approximately 100% at a rate of between approximately 1% and approximately 88%. In certain embodiments, the method of treatment is effective in obtaining in the subject a reduction in tumor burden of between approximately 90% and approximately 100% at a rate of between approximately 1% and approximately 88%. In certain embodiments, the method of treatment is effective in obtaining in the subject a reduction in tumor burden of between approximately 95% and approximately 100% at a rate of between approximately 1% and approximately 88%. In certain embodiments, the method of treatment is effective in obtaining in the subject a reduction in tumor burden of between approximately 99% and approximately 100% at a rate of between approximately 1% and approximately 88%. In certain embodiments, the method of treatment is effective in obtaining in the subject a reduction in tumor burden of approximately 100% at a rate of between approximately 1% and approximately 83%.

In certain embodiments, the method of treatment is effective in obtaining in the subject minimal residual disease (MRD) negativity or maintaining said minimal residual disease (MRD) negative status.

In certain embodiments, the method of treatment is effective in obtaining in the subject minimal residual disease (MRD) negativity. In certain embodiments, the method of treatment is effective in obtaining in the subject a minimal residual disease (MRD) negativity at a sensitivity level of 10⁻⁶. In certain embodiments, the method of treatment is effective in obtaining in the subject minimal residual disease (MRD) negativity at a sensitivity level of 10⁻⁵. In certain embodiments, the method of treatment is effective in obtaining in the subject minimal residual disease (MRD) negativity at a sensitivity level of 10⁻⁴. In certain embodiments, the method of treatment is effective in obtaining in the subject minimal residual disease (MRD) negativity at a sensitivity level of 10⁻³. In certain embodiments, the method of treatment is effective in obtaining MRD negativity when assessed in the bone marrow. In certain embodiments, the method of treatment is effective in maintaining the MRD negativity when assessed using a bone marrow sample that is evaluable. In certain embodiments, the method of treatment is effective in obtaining MRD negativity when assessed using bone marrow DNA. In certain embodiments, the MRD negativity is assessed using next generation sequencing (NGS) or next generation flow (NGF) on bone marrow aspirate DNA of the subject.

In some embodiments, the method is effective in obtaining minimal residual disease (MRD) negativity in the subject assessed in the bone marrow at a follow-up time of about 0.9 month to about 6.01 months after the administering of the CAR-T cells. In some embodiments, the method is effective in obtaining minimal residual disease (MRD) negativity in the subject assessed in the bone marrow at a follow-up time of about 0.5 month after the administering of the CAR-T cells, about 0.6 month or later after the administering of the CAR-T cells, about 0.7 month or later after the administering of the CAR-T cells, about 0.8 month or later after the administering of the CAR-T cells, about 0.9 month or later after the administering of the CAR-T cells, about 1.0 month or later after the administering of the CAR-T cells, about 1.2 months or later after the administering of the CAR-T cells, about 1.4 months or later after the administering of the CAR-T cells, about 1.8 months or later after the administering of the CAR-T cells, about 0.6 month or later after the administering of the CAR-T cells, about 2.0 months or later after the administering of the CAR-T cells, about 2.4 months or later after the administering of the CAR-T cells, about 2.6 months or later after the administering of the CAR-T cells, about 2.8 months or later after the administering of the CAR-T cells, about 3.0 months or later after the administering of the CAR-T cells, about 3.4 months or later after the administering of the CAR-T cells, about 3.8 months or later after the administering of the CAR-T cells, about 4.2 months or later after the administering of the CAR-T cells, about 4.6 months or later after the administering of the CAR-T cells, about 5.0 months or later after the administering of the CAR-T cells, about 5.2 months or later after the administering of the CAR-T cells, about 5.4 months or later after the administering of the CAR-T cells, about 5.6 months or later after the administering of the CAR-T cells, about 6.0 months or later after the administering of the CAR-T cells, about 6.2 months or later after the administering of the CAR-T cells, about 6.4 months or later after the administering of the CAR-T cells, about 6.6 months or later after the administering of the CAR-T cells, about 6.8 months or later after the administering of the CAR-T cells, about 7.0 months or later after the administering of the CAR-T cells, about 7.5 months or later after the administering of the CAR-T cells, about 8.0 months or later after the administering of the CAR-T cells, about 8.5 months or later after the administering of the CAR-T cells.

In some embodiments, the minimal residual disease (MRD) negativity is obtained at a median time of about 0.5, about 0.6, about 0.7, about 0.8, about 0.9, about 1.0, about 1.1, about 1.2, about 1.3, about 1.4, about 1.5, about 1.6, about 1.8, about 2.0, about 2.5, about 3.0, about 3.5, about 4.0, about 4.5, about 5.0, about 5.5, or about 6.0 months after the administering to the subject the dose of the T cells. In some embodiments, the minimal residual disease (MRD) negativity is obtained at a median time of about 1.33 months after the administering to the subject the dose of the T cells.

In some embodiments, the minimal residual disease (MRD) negativity is obtained at a mean time of about 0.5, about 0.6, about 0.7, about 0.8, about 0.9, about 1.0, about 1.1, about 1.2, about 1.3, about 1.4, about 1.5, about 1.6, about 1.8, about 2.0, about 2.1, about 2.2, about 2.3, about 2.4, about 2.5, about 3.0, about 3.5, about 4.0, about 4.5, about 5.0, about 5.5, or about 6.0 months after the administering to the subject the dose of the T cells. In some embodiments, the minimal residual disease (MRD) negativity is obtained at a mean time of about 2.10 months after the administering to the subject the dose of the T cells.

In some embodiments, the method is effective in obtaining the MRD negativity at a rate of between about 44.0% to about 89.7% at a sensitivity of 10-5. In some embodiments, the method is effective in obtaining the MRD negativity at a rate of about 70.6% at a sensitivity of 10⁻⁵. In some embodiments, the method is effective in obtaining the MRD negativity at a rate of between about 51.9% to about 95.7% at a sensitivity of 10⁻⁵. In some embodiments, the method is effective in obtaining the MRD negativity at a rate of about 80.0% at a sensitivity of 10⁻⁵.

In some embodiments, the method is effective in obtaining at least one response in the subject after the administering to the subject the dose of the T cells, and wherein the at least one response comprises, in order from better to worse:
(i) a stringent complete response;
(ii) a complete response;
(iii) a very good partial response;
(iv) a partial response; or
(v) a minimal response.

In some embodiments, the method is effective in obtaining a first response of any one of a partial response, a very good partial response, a complete response, or a stringent complete response. In some embodiments, the method is effective in obtaining the first response at a time of between about 0.9 month and about 12.5 months after the administering of the dose of the T cells. In some embodiments, the method is effective in obtaining the first response at a mean time of about 3.07 months after the administering of the dose of the T cells. In some embodiments, the method is effective in obtaining a first response at a median time of about 1.30 months after the administering of the dose of the T cells.

In some embodiments, the method is effective in obtaining a best response of any one of a minimal response, a partial response, a very good partial response, a complete response, or a stringent complete response. In some embodiments, the method is effective in obtaining the best response at a time of between about 0.9 month and about 12.5 months after the administering of the dose of the T cells. In some embodiments, the method is effective in obtaining the best response at a mean time of about 4.02 months after the administering of the dose of the T cells. In some embodiments, the method is effective in obtaining the best response at a median time of about 1.89 months after the administering of the dose of the T cells.

In some embodiments, the method is effective in obtaining a complete response or a stringent complete response at a time of between about 0.9 month and about 12.5 months after the administering of the dose of the T cells. In some embodiments, the method is effective in obtaining the complete response or the stringent complete response at a mean time of about 3.30 months after the administering of the dose of the T cells. In some embodiments, the method is effective in obtaining the complete response or the stringent complete response at a median time of about 1.72 months after the administering of the dose of the T cells. In some embodiments, the method is effective in obtaining a complete response or a stringent complete response at a rate of between about 71.3% and about 99.9%. In some embodiments, the method is effective in obtaining the complete response or the stringent complete response at a rate of about 94.1%.

In some embodiments, the method is effective in obtaining a very good partial response, a complete response or a stringent complete response at a rate of between about 71.3% and about 99.9%. In some embodiments, the method is effective in obtaining the very good partial response, the complete response or the stringent complete response at a rate of about 94.1%.

In some embodiments, the method is effective in obtaining a partial response, a very good partial response, a complete response or a stringent complete response at a rate of between about 71.3% and about 99.9%. In some embodiments, the method is effective in obtaining the partial response, the very good partial response, the complete response or the stringent complete response at a rate of about 94.1%.

In some embodiments, the method is effective in obtaining a minimal response, a partial response, a very good partial response, a complete response or a stringent complete response at a rate of between about 71.3% and about 99.9%. In some embodiments, the method is effective in obtaining the minimal response, the partial response, the very good partial response, the complete response or the stringent complete response at a rate of about 94.1%.

In some embodiments, the method is effective in obtaining a best response comprising a stringent complete response at a rate of between about 63.6% and about 98.5%. In some embodiments, the method is effective in obtaining the stringent complete response at a rate of about 88.2%.

In some embodiments, the method is effective in obtaining a best response comprising a complete response or a stringent complete response and further comprising a MRD negativity at a rate of between about 50.1% and about 93.2%. In some embodiments, the method is effective in obtaining the best response at a rate of about 76.5%.

In some embodiments, the method is effective in obtaining a best response comprising a complete response at a rate of between about 0.1% and about 28.7%, wherein optionally the method is effective in obtaining the best response at a rate of about 5.9%.

In some embodiments, the method is effective in maintaining the response for at least about 6 months at a rate of about 100%. In some embodiments, the method is effective in maintaining the response for at least about 9 months at a rate of about 100%. In some embodiments, the method is effective in maintaining the response for at least about 12 months at a rate of about 100%.

In some embodiments, the method is effective in obtaining progression-free survival of the subject. In some embodiments, the method is effective in obtaining the progression-free survival at a rate of about 100.0% at a follow-up time of about 6 months. In some embodiments, the method is effective in obtaining the progression-free survival at a rate of about 100.0% at a follow-up time of about 9 months. In some embodiments, the method is effective in obtaining the progression-free survival at a rate of between about 63.2% and about 99.1% at a follow-up time of about 12 months. In some embodiments, the method is effective in obtaining the progression-free survival at a rate of about 93.8% at a follow-up time of about 12 months. In some embodiments, the method is effective in obtaining the progression-free survival at a rate of between about 63.2% and about 99.1% at a follow-up time of about 18 months. In some embodiments, the method is effective in obtaining the progression-free survival at a rate of about 93.8% at a follow-up time of about 18 months.

In some embodiments, the method is effective in obtaining overall survival of the subject. In some embodiments, the method is effective in obtaining the overall survival at a rate of between about 90.0% and about 100.0% at a follow-up time of about 6 months. In some embodiments, the method is effective in obtaining the overall survival at a rate of about 100.0% at a follow-up time of about 6 months. In some embodiments, the method is effective in obtaining the overall survival at a rate of between about 90.0% and about 100.0% at a follow-up time of about 9 months. In some embodiments, the method is effective in obtaining the overall survival at a rate of about 100.0% at a follow-up time of about 9 months. In some embodiments, the method is effective in obtaining the overall survival at a rate of between about 63.2% and about 99.1% at a follow-up time of about 12 months. In some embodiments, the method is effective in obtaining the overall survival at a rate of about 93.8% at a follow-up time of about 12 months. In some embodiments, the method is effective in obtaining the overall survival at a rate of between about 63.2% and about 99.1% at a follow-up time of about 18 months. In some embodiments, the method is effective in obtaining the overall survival at a rate of about 93.8% at a follow-up time of about 18 months.

In some embodiments, the method further comprising treating the subject for an adverse event after the administering of the dose of the T cells. In some embodiments, the method comprises administering a treatment to the subject to alleviate the adverse event.

In some embodiments, the adverse event comprises a treatment-emergent adverse event. In some embodiments, the treatment-emergent adverse event is a serious treatment-emergent adverse event.

In some embodiments, the treatment-emergent adverse event occurs within the later of about 100 days at or after the administering of the dose of the T cells or about 30 days after last dose of lenalidomide.

In some embodiments, severity of the treatment-emergent adverse event is Grade 1, Grade 2, Grade 3, or Grade 4. In some embodiments, the severity of the treatment-emergent adverse event is Grade 3 or 4, wherein optionally maximum severity of treatment-emergent adverse event is Grade 4. In some embodiments, maximum severity of the treatment-emergent adverse event is Grade 3. In some embodiments, the maximum severity of the treatment-emergent adverse event of Grade 3 occurs at a rate of about 17.6%. In some embodiments, maximum severity of the treatment-emergent adverse event is Grade 4. In some embodiments, the maximum severity of the treatment-emergent adverse event of Grade 4 occurs at a rate of about 82.4%.

In some embodiments, the treatment-emergent adverse event comprises a blood or lymphatic system disorder, an immune system disorder, a gastrointestinal disorder, an infection, an infestation, a musculoskeletal or connective tissue disorder, a respiratory, thoracic or mediastinal disorder, a general disorder, an administrate site condition, a metabolism or nutrition disorder, a nervous system disorder, a skin or subcutaneous tissue disorder, an eye disorder, a cardiac disorder, an ear or labyrinth disorder, or a vascular disorder. In some embodiments, the treatment-emergent adverse event occurs at a rate of at least about 10%.

In some embodiments, the treatment-emergent adverse event comprises neutropenia, lymphopenia, thrombocytopenia, leukopenia, anemia, febrile neutropenia, cytokine release syndrome, hypogammaglobulinaemia, diarrhoea, nausea, abdominal distension, abdominal pain, upper abdominal pain, constipation, dyspepsia, vomiting, increased aspartate aminotransferase, increased alanine aminotransferase, increased blood lactate dehydrogenase, decreased CD4 lymphocytes, increased gamma-glutamyltransferase, decreased serum ferritin, an upper respiratory tract infection, COVID-19, nasopharyngitis, pneumonia, respiratory syncytial virus infection, Rhinovirus infection, sinusitis, myalgia, back pain, muscle spasm, musculoskeletal stiffness, pain in extremity, cough, productive cough, nasal congestion, rhinorrhoea, epistaxis, oropharyngeal pain, fatigue, chill, malaise, pyrexia, hyperferritinaemia, hypoalbuminaemia, hypocalcaemia, hypomagnesaemia, hypophosphataemia, headache, dysarthria, hyperhidrosis, pruritus, or sinus tachycardia. In some embodiments, the treatment-emergent adverse event occurs at a rate of at least about 10%.

In some embodiments, the treatment-emergent adverse event is a serious treatment-emergent adverse event that occurs at a rate of about 58.8%. In some embodiments, maximum severity of the treatment-emergent adverse event is Grade 3 or Grade 4. In some other embodiments, the maximum severity of the treatment-emergent adverse event of Grade 3 or Grade 4 occurs at a rate of about 52.9%.

In some embodiments, the serious treatment-emergent adverse event comprises an infection, an infestation, a nervous system disorder, a blood or lymphatic system disorder, an immune system disorder, an eye disorder, a gastrointestinal disorder, a musculoskeletal or connective tissue disorder, or a vascular disorder. In some embodiments, the serious treatment-emergent adverse event occurs at a rate of at least about 2%.

In some embodiments, the serious treatment-emergent adverse event comprises pneumonia, Rhinovirus infection, COVID-19 pneumonia, metapneumovirus infection, influenzal pneumonia, viral pneumonia, respiratory syncytial virus infection, sinusitis, streptococcal sepsis, facial nerve disorder, facial paralysis, peripheral motor neuropathy, febrile neutropenia, neutropenia, cytokine release syndrome, diplopia, diarrhoea, musculoskeletal pain, or haematoma. In some embodiments, the serious treatment-emergent adverse event occurs at a rate of at least about 2%.

In some embodiments, the treatment-emergent adverse event comprises cytokine release syndrome (CRS), wherein optionally the CRS occurs at a rate of about 82.4%, wherein optionally:
(1) time to first onset of the CRS ranges from about 6 to about 11 days after the administering of the dose of the T cells to the subject, wherein further optionally the time to the first onset of the CRS is at a median of about 8.0 days or at a mean of about 8.1 days;
(2) time to recovery of the CRS ranges from about 1 day to about 5 days, wherein further optionally the time to recovery of the CRS is at a median time of about 2.5 days or at a mean time of about 2.6 days;
(3) duration of the CRS ranges from about 1 day to about 5 days or from about 2 days to about 3 days, wherein further optionally the duration of the CRS is at a median time of about 2.5 days or at a mean time of about 2.6 days; or
(4) duration of the CRS of less than about 7 days occurs at a rate of about 100%.

In some embodiments, the CRS occurs at maximum toxicity grade of Grade 1, Grade 2, Grade 3, or Grade 4, wherein optionally:
(1) the CRS occurs at the maximum toxicity grade of Grade 1 at a rate of about 76.5%; or
(2) the CRS occurs at the maximum toxicity grade of Grade 2 at a rate of about 5.9%.

In some embodiments, the treatment comprises an anti-IL-6 receptor, an IL-1 receptor antagonist, a corticosteroid, IV fluids, a vasopressor, oxygen, an analgesic, an anti-inflammatory drug, an antiinfective, an antiepileptic, caffeine, or prochlorperazine, or any combination thereof, wherein optionally:
(1) the anti-IL-6 receptor comprises tocilizumab;
(2) the IL-1 receptor antagonist comprises anakinra; or
(3) oxygen comprises blow-by, nasal cannula low flow at a flow rate of at most about 6 L/min, nasal cannula high flow at a flow rate of at least about 6 L/min, face mask, non-rebreather mask, venturi mask, or positive pressure, or any combination thereof.

In some embodiments, the CRS is recovered or resolved, wherein optionally the CRS is recovered or resolved at a rate of about 100%.

In some embodiments, the treatment-emergent adverse event comprises immune effector cell-associated neurotoxicity (ICANS), wherein optionally the ICANS occurs at a rate of about 5.9%, and wherein optionally:
(1) time to first onset of the ICANS is about 7 days after the administering of the dose of the T cells to the subject, wherein further optionally the time to the first onset of the ICANS is at a median of about 7 days or at a mean of about 7 days;
(2) time to recovery of the ICANS is about 1 day, wherein further optionally the time to recovery of the ICANS is at a median time of about 1 day or at a mean time of about 1 day;
(3) duration of the ICANS is about 1 day, wherein further optionally the duration of the ICANS is at a median time of about 1 day or at a mean time of about 1 day; or
(4) the ICANS occurs concurrently with cytokine release syndrome (CRS) at a rate of about 5.9%.

In some embodiments, the ICANS occurs at maximum toxicity grade of Grade 1, Grade 2, Grade 3, Grade 4, or Grade 5. In some embodiments, the ICANS occurs at maximum toxicity grade of Grade 1 at a rate of about 5.9%.

In some embodiments, the treatment comprises an anti-IL-6 receptor, an IL-1 receptor antagonist, a corticosteroid, or ceftazidime, or any combination thereof, wherein optionally:
(1) the anti-IL-6 receptor comprises tocilizumab; or
(2) the IL-1 receptor antagonist comprises anakinra.

In some embodiments, the ICANS is recovered, resolved, not recovered, not resolved, recovered with sequelae, resolved with sequelae, recovering, resolving, fatal, or unknown, wherein optionally the ICANS is recovered or resolved at a rate of about 100%.

In some embodiments, the adverse event comprises neurotoxicity, wherein optionally the neurotoxicity occurs at a rate of about 35.3%, wherein optionally:
(1) time to first onset of the neurotoxicity ranges from about 16 days to about 27 days after the administering of the dose of the T cells to the subject, wherein further optionally the time to the first onset of the neurotoxicity is at a median of about 21.0 days or at a mean of about 20.8 days;
(2) time to recovery of the neurotoxicity ranges from about 29 days to about 443 days, wherein further optionally the time to recovery of the neurotoxicity is at a median time of about 70.0 days or at a mean time of about 153.0 days;
(3) duration of the neurotoxicity ranges from about 29 days to about 791 days, wherein further optionally the duration of the neurotoxicity is at a median time of about 111.0 days or at a mean time of about 254.7 days.

In some embodiments, the neurotoxicity is recovered, resolved, not recovered, or not resolved, wherein optionally the neurotoxicity is recovered or resolved at a rate of about 23.5%, and wherein optionally the neurotoxicity is not recovered or not resolved at a rate of about 11.8%.

In some embodiments, the adverse event is an adverse event of special interest, wherein optionally:
(1) the adverse event of special interest occurs at a rate of about 82.4%, wherein further optionally the adverse event of special interest occurs at least at Grade 3 at a rate of about 23.5%; or
(2) the adverse event of special interest comprises cytokine release syndrome (CRS), CAR-T cell related neurotoxicity, second primary malignancy, or a movement and neurocognitive treatment-emergent adverse event, or any combination thereof.

In some embodiments, the adverse event of special interest is CRS, wherein optionally the CRS occurs at a rate of about 82.4%, wherein further optionally the CRS occurs at least at Grade 3 at a rate of about 0%.

In some embodiments, the adverse event of special interest is CAR-T cell related neurotoxicity, wherein optionally:
(1) the CAR-T cell related neurotoxicity occurs at a rate of about 35.3%, wherein further optionally the CAR-T cell related neurotoxicity occurs at least at Grade 3 at a rate of about 5.9%; or
(2) the CAR-T cell related neurotoxicity comprises immune effector cell-associated neurotoxicity (ICANS) or other neurotoxicity, wherein further optionally the ICANS occurs at a rate of about 5.9%, wherein further optionally the ICANS occurs at least at Grade 3 at a rate of about 0%, wherein further optionally the other neurotoxicity occurs at a rate of about 35.3%, and wherein further optionally the other neurotoxicity occurs at least at Grade 3 at a rate of about 5.9%.

In some embodiments, the adverse event of special interest is second primary malignancy, wherein optionally the second primary malignancy comprises myelodysplastic syndrome, wherein optionally the second primary malignancy occurs at a rate of about 5.9%, wherein further optionally the second primary malignancy occurs at least at Grade 3 at a rate of about 5.9%.

In some embodiments, the adverse event of special interest is a movement and neurocognitive treatment-emergent adverse event, wherein optionally the movement and neurocognitive treatment-emergent adverse event does not occur in the subject.

In some embodiments, the adverse event comprises prolonged cytopenia, wherein optionally the prolonged cytopenia comprises thrombocytopenia, neutropenia, lymphopenia, or anemia, or any combination thereof, wherein further optionally:
(1) thrombocytopenia occurs at Grade 3 or 4 at a rate of about 29.4% after the administering of the dose of the T cells to the subject, wherein further optionally thrombocytopenia recovers to Grade 2 or less by about 30 days or about 60 days, and wherein further optionally thrombocytopenia reoccurs at Grade 3 or 4 at a rate of about 0%;
(2) neutropenia occurs at Grade 3 or 4 at a rate of about 88.2% after the administering of the dose of the T cells to the subject, wherein further optionally neutropenia recovers to Grade 2 or less by about 30 days or about 60 days, and wherein further optionally neutropenia reoccurs at Grade 3 or 4 at a rate of about 35.3%;
(3) lymphopenia occurs at Grade 3 or 4 at a rate of about 100% after the administering of the dose of the T cells to the subject, wherein further optionally lymphopenia recovers to Grade 2 or less by about 30 days or about 60 days, and wherein further optionally lymphopenia reoccurs at Grade 3 or 4 at a rate of about 11.8%; or
(4) anemia occurs at Grade 3 or 4 at a rate of about 5.9% after the administering of the dose of the T cells to the subject, wherein further optionally anemia recovers to Grade 2 or less by about 30 days or about 60 days, and wherein further optionally anemia reoccurs at Grade 3 or 4 at a rate of about 0%.

In some embodiments, the treatment-emergent adverse event comprises a treatment-emergent infection, wherein optionally:
(1) the treatment-emergent infection occurs at a rate of about 70.6%, wherein further optionally the treatment-emergent infection occurs at Grade 3 or 4 at a rate of about 29.4%;
(2) the treatment-emergent infection comprises an infection, an infestation, a viral infectious disorder, a bacterial infectious disorder, or a fungal infectious disorder, or any combination thereof,
(3) the treatment-emergent infection comprises an upper respiratory tract infection, nasopharyngitis, pneumonia, sinusitis, acute sinusitis, bronchitis, infectious enterocolitis, gastroenteritis, pharyngitis, COVID-19, respiratory syncytial virus infection, Rhinovirus infection, COVID-19 pneumonia, herpes zoster, influenza, metapneumovirus infection, oral herpes, influenzal pneumonia, viral pneumonia, Campylobacter infection, Enterococcal infection, bacterial respiratory tract infection, Streptococcal sepsis, Aspergillus infection, Candida infection, fungal foot infection, or tongue fungal infection, or any combination thereof.

In certain embodiments, the efficacy of the method of treatment is assessed by evaluating the proportion of subjects with MRD negative status. In certain embodiments, the efficacy of the method of treatment is assessed by evaluating the proportion of subjects with MRD negative status at a sensitivity level of 10⁻⁶. In certain embodiments, the efficacy of the method of treatment is assessed by evaluating the proportion of subjects with MRD negative status at a sensitivity level of 10⁻⁵. In certain embodiments, the efficacy of the method of treatment is assessed by evaluating the proportion of subjects with MRD negative status at a sensitivity level of 10⁻⁴. In certain embodiments, the efficacy of the method of treatment is assessed by evaluating the proportion of subjects with MRD negative status at a sensitivity level of 10⁻³.

In certain embodiments, the efficacy of the method of treatment is assessed by evaluating the proportion of subjects with evaluable bone marrow and MRD negative status. In certain embodiments, the efficacy of the method of treatment is assessed by evaluating the proportion of subjects with evaluable bone marrow and MRD negative status at a sensitivity level of 10⁻⁶. In certain embodiments, the efficacy of the method of treatment is assessed by evaluating the proportion of subjects with evaluable bone marrow and MRD negative status at a sensitivity level of 10⁻⁵. In certain embodiments, the efficacy of the method of treatment is assessed by evaluating the proportion of subjects with evaluable bone marrow and MRD negative status at a sensitivity level of 10⁻⁴. In certain embodiments, the efficacy of the method of treatment is assessed by evaluating the proportion of subjects with evaluable bone marrow and MRD negative status at a sensitivity level of 10⁻³.

Also provided in further aspects is the medical use of the therapies (including CARs, CAR T cells) provided herein for use in the methods of treatment disclosed herein. Also provided in further aspects is the use of the therapies (including CARs, and CAR T cells) provided herein for use in the manufacture of a medicament for the use in the methods of treatment disclosed herein.

### 4.9. Kits and Articles of Manufacture

Any of the compositions described herein may be comprised in a kit. In some embodiments, engineered immortalized CAR-T cells are provided in the kit, which also may include reagents suitable for expanding the cells, such as media.

In a non-limiting example, a chimeric receptor expression construct, one or more reagents to generate a chimeric receptor expression construct, cells for transfection of the expression construct, and/or one or more instruments to obtain immortalized T cells for transfection of the expression construct (such an instrument may be a syringe, pipette, forceps, and/or any such medically approved apparatus).

In some embodiments, the kit comprises reagents or apparatuses for electroporation of cells.

In some embodiments, the kit comprises artificial antigen presenting cells.

The kits may comprise one or more suitably aliquoted compositions of the present disclosure or reagents to generate compositions of the disclosure. The components of the kits may be packaged either in aqueous media or in lyophilized form. The container means of the kits may include at least one vial, test tube, flask, bottle, syringe, or other container means, into which a component may he placed, and preferably, suitably aliquoted. Where there is more than one component in the kit, the kit also will generally contain a second, third, or other additional container into which the additional components may be separately placed. However, various combinations of components may be comprised in a vial. The kits of the present disclosure also will typically include a means for containing the chimeric receptor construct and any other reagent containers in close confinement for commercial sale. Such containers may include injection or blow molded plastic containers into which the desired vials are retained, for example.

### 4.10. Particular Embodiments

Particular embodiments of the disclosure are set forth in the following numbered paragraphs:

1. A method of treating a subject with multiple myeloma, wherein the subject has not achieved a complete response after receiving an initial therapy comprising a stem cell transplantation, the method comprising administering to the subject a dose of T cells comprising a chimeric antigen receptor (CAR) comprising:
(a) an extracellular antigen binding domain capable of specifically binding to an epitope of B-cell maturation antigen (BCMA), wherein the extracellular antigen binding domain comprises a first VHH domain and a second VHH domain, and wherein the first VHH domain comprising a CDR1, a CDR2, and a CDR3 as set forth in the VHH domain comprising the amino acid sequence of SEQ ID NO: 2, and the second VHH domain comprising a CDR1, a CDR2, and a CDR3 as set forth in the VHH domain comprising the amino acid sequence of SEQ ID NO: 4,
(b) a transmembrane domain, and
(c) an intracellular signaling domain.

2. The method of embodiment 1, wherein the initial therapy further comprises (1) 4 to 8 cycles of an induction therapy and (2) a high-dose chemotherapy.

3. The method of embodiment 1 or 2, wherein the stem cell transplantation is autologous or allogenic stem cell transplantation.

4. The method of embodiment 3, wherein the stem cell transplantation is autologous stem cell transplantation (ASCT) or tandem ASCT.

5. The method of any one of embodiments 2-4, wherein the high-dose chemotherapy comprises melphalan.

6. The method of any one of embodiments 2-5, wherein the induction therapy comprises a proteasome inhibitor (PI) and an immunomodulatory drug (IMiD).

7. The method of embodiment 6, wherein the PI is bortezomib, carfilzomib, or ixazomib.

8. The method of embodiment 6, wherein the IMiD is lenalidomide, pomalidomide or thalidomide.

9. The method of any one of embodiments 6-8, wherein the induction therapy further comprises an alkylating agent.

10. The method of embodiment 9, wherein the alkylating agent is cyclophosphamide.

11. The method of any one of embodiments 6-10, wherein the induction therapy further comprises an anti-CD38 antibody.

12. The method of any one of embodiments 1-11, further comprising administering to the subject a dose of an immunomodulatory drug (IMiD) after administering to the subject the dose of the T cells.

13. The method of embodiment 12, wherein the subject is not refractory to the IMiD administered after the dose of the T cells.

14. The method of embodiments 12 or 13, wherein the IMiD is lenalidomide.

15. The method of embodiment 14, wherein the dose of lenalidomide is about 2.5 mg, about 5 mg, about 10 mg, or about 15 mg daily.

16. The method of embodiment 15, wherein the dose of lenalidomide is about 2.5 mg daily.

17. The method of embodiment 15, wherein the dose of lenalidomide is about 5 mg daily.

18. The method of embodiment 15, wherein the dose of lenalidomide is about 10 mg daily.

19. The method of embodiment 15, wherein the dose of lenalidomide is about 15 mg daily.

20. The method of any one of embodiments 14-19, wherein the administering of the dose of lenalidomide is once daily.

21. The method of any one of embodiments 14-20, wherein the administering of the dose of lenalidomide starts between about 21 days to about 214 days after the administering of the dose of the T cells to the subject.

22. The method of embodiment 21, wherein the administering of the dose of lenalidomide starts at a median of about 51 days or at a mean of about 85.1 days.

23. The method of embodiment 21 or 22, wherein the administering of the dose of lenalidomide continues for between about 70 days to about 716 days.

24. The method of embodiment 23, wherein the administering of the dose of lenalidomide continues for a median of about 426.5 days or at a mean of about 426.0 days.

25. The method of any one of embodiments 14-24, wherein the administering of the dose of lenalidomide is daily in a cycle of about 28 days.

26. The method of embodiment 25, wherein the administering of the dose of lenalidomide continues for between about 3 cycles to about 26 cycles.

27. The method of embodiment 26, wherein the administering of the dose of the lenalidomide continues for a median or a mean of about 15 cycles.

28. The method of any one of embodiments 25-27, wherein the administering of the dose of the lenalidomide is at a relative dose intensity of between about 67.9% to about 100.0%.

29. The method of embodiment 28, wherein the administering of the dose of the lenalidomide is at a median relative dose intensity of about 93.4%.

30. The method of any one of embodiments 14-29, wherein the administering of the dose of lenalidomide continues until the later of when the subject is confirmed progressive disease or unacceptable toxicity, or when it has reached 2 years after start of the administering of the dose of lenalidomide.

31. The method of any one of embodiments 1-30, wherein the subject has further received a lymphodepletion therapy at least about 5 to about 7 days prior to the administering of the dose of the T cells.

32. The method of embodiment 31, wherein the lymphodepletion therapy comprises administering cyclophosphamide and fludarabine daily, and optionally wherein the lymphodepletion therapy comprises cyclophosphamide at a concentration of about 300 mg/m² and fludarabine at a concentration of about 30 mg/m² daily for 3 days.

33. The method of embodiment 31 or 32, wherein the subject has further received a bridging therapy prior to the lymphodepletion therapy, wherein optionally the bridging therapy comprises at least one cycle of lenalidomide at a dose of 10 mg per day.

34. The method of any one of embodiments 1-33, wherein the dose of the T cells is about 0.5-1.0×10⁶ of the T cells/kg of body weight of the subject.

35. The method of embodiment 34, wherein the dose of the T cells is about 0.56-0.84×10⁶ of the T cells/kg of body weight of the subject.

36. The method of embodiment 35, wherein the dose of the T cells is about 0.68×10⁶ of the T cells/kg of body weight of the subject.

37. The method of embodiment 34, wherein the dose of the T cells is formulated to be about 0.60-0.80×10⁶ of the T cells/kg of body weight of the subject.

38. The method of embodiment 37, wherein the dose of the T cells is formulated to be about 0.70×10⁶ of the T cells/kg of body weight of the subject.

39. The method of any one of embodiments 1-38, wherein the administering of the dose of the T cells is in a single, two, or three infusions.

40. The method of any one of embodiments 1-39, wherein the method is effective in obtaining minimal residual disease (MRD) negativity assessed in the bone marrow of the subject after the administering to the subject the dose of the T cells.

41. The method of embodiment 40, wherein the MRD negativity is assessed using next generation sequencing (NGS) or next generation flow (NGF) on bone marrow aspirate DNA of the subject, wherein optionally the MRD negativity is assessed at a sensitivity of 10⁻⁵.

42. The method of embodiment 40 or 41, wherein the method is effective in obtaining MRD negativity at about 0.9 month to about 6.1 months after the administering to the subject the dose of the T cells.

43. The method of embodiment 42, wherein the method is effective in obtaining MRD negativity at a median time of about 1.33 months after the administering to the subject the dose of the T cells.

44. The method of embodiment 42, wherein the method is effective in obtaining MRD negativity at a mean time of about 2.10 months after the administering to the subject the dose of the T cells.

45. The method of any one of embodiments 40-44, wherein the method is effective in obtaining the MRD negativity at a rate of between about 44.0% to about 89.7% at a sensitivity of 10-5.

46. The method of embodiment 45, wherein the method is effective in obtaining the MRD negativity at a rate of about 70.6% at a sensitivity of 10⁻⁵.

47. The method of any one of embodiments 40-44, wherein the method is effective in obtaining the MRD negativity at a rate of between about 51.9% to about 95.7% at a sensitivity of 10⁻⁵.

48. The method of embodiment 47, wherein the method is effective in obtaining the MRD negativity at a rate of about 80.0% at a sensitivity of 10⁻⁵.

49. The method of any one of embodiments 1-48, wherein the method is effective in obtaining at least one response in the subject after the administering to the subject the dose of the T cells, and wherein the at least one response comprises, in order from better to worse:
(i) a stringent complete response;
(ii) a complete response;
(iii) a very good partial response;
(iv) a partial response; or
(v) a minimal response.

50. The method of embodiment 49, wherein the method is effective in obtaining a first response of any one of a partial response, a very good partial response, a complete response, or a stringent complete response.

51. The method of embodiment 50, wherein the method is effective in obtaining the first response at a time of between about 0.9 months and about 12.5 months after the administering of the dose of the T cells.

52. The method of embodiment 51, wherein the method is effective in obtaining the first response at a mean time of about 3.07 months after the administering of the dose of the T cells.

53. The method of embodiment 51, wherein the method is effective in obtaining the first response at a median time of about 1.30 months after the administering of the dose of the T cells.

54. The method of embodiment 49, wherein the method is effective in obtaining a best response of any one of a partial response, a very good partial response, a complete response, or a stringent complete response.

55. The method of embodiment 54, wherein the method is effective in obtaining the best response at a time of between about 0.9 months and about 12.5 months after the administering of the dose of the T cells.

56. The method of embodiment 55, wherein the method is effective in obtaining the best response at a mean time of about 4.02 months after the administering of the dose of the T cells.

57. The method of embodiment 55, wherein the method is effective in obtaining the best response at a median time of about 1.89 months after the administering of the dose of the T cells.

58. The method of embodiment 49, wherein the method is effective in obtaining a complete response or a stringent complete response at a time of between about 0.9 months and about 12.5 months after the administering of the dose of the T cells.

59. The method of embodiment 58, wherein the method is effective in obtaining the complete response or the stringent complete response at a mean time of about 3.30 months after the administering of the dose of the T cells.

60. The method of embodiment 58, wherein the method is effective in obtaining the complete response or the stringent complete response at a median time of about 1.72 months after the administering of the dose of the T cells.

61. The method of embodiment 49, wherein the method is effective in obtaining a complete response or a stringent complete response at a rate of between about 71.3% and about 99.9%.

62. The method of embodiment 61, wherein the method is effective in obtaining the complete response or the stringent complete response at a rate of about 94.1%.

63. The method of embodiment 49, wherein the method is effective in obtaining a very good partial response, a complete response or a stringent complete response at a rate of between about 71.3% and about 99.9%.

64. The method of embodiment 63, wherein the method is effective in obtaining the very good partial response, the complete response or the stringent complete response at a rate of about 94.1%.

65. The method of embodiment 49, wherein the method is effective in obtaining a partial response, a very good partial response, a complete response or a stringent complete response at a rate of between about 71.3% and about 99.9%.

66. The method of embodiment 65, wherein the method is effective in obtaining the partial response, the very good partial response, the complete response or the stringent complete response at a rate of about 94.1%.

67. The method of embodiment 49, wherein the method is effective in obtaining a minimal response, a partial response, a very good partial response, a complete response or a stringent complete response at a rate of between about 71.3% and about 99.9%.

68. The method of embodiment 67, wherein the method is effective in obtaining the minimal response, the partial response, the very good partial response, the complete response or the stringent complete response at a rate of about 94.1%.

69. The method of embodiment 49, wherein the method is effective in obtaining a best response comprising a stringent complete response at a rate of between about 63.6% and about 98.5%.

70. The method of embodiment 69, wherein the method is effective in obtaining the stringent complete response at a rate of about 88.2%.

71. The method of embodiment 49, wherein the method is effective in obtaining a best response comprising a complete response or a stringent complete response and further comprising a MRD negativity at a rate of between about 50.1% and about 93.2%, wherein optionally the method is effective in obtaining the best response at a rate of about 76.5%.

72. The method of embodiment 49, wherein the method is effective in obtaining a best response comprising a complete response at a rate of between about 0.1% and about 28.7%, wherein optionally the method is effective in obtaining the best response at a rate of about 5.9%.

73. The method of embodiment 49, wherein the method is effective in maintaining the response for at least about 6 months at a rate of about 100%.

74. The method of embodiment 73, wherein the method is effective in maintaining the response for at least about 9 months at a rate of about 100%.

75. The method of embodiment 73, wherein the method is effective in maintaining the response for at least about 12 months at a rate of about 100%.

76. The method of any one of embodiments 1-48, wherein the method is effective in obtaining progression-free survival of the subject.

77. The method of embodiment 76, wherein the method is effective in obtaining the progression-free survival at a rate of about 100.0% at a follow-up time of about 6 months.

78. The method of embodiment 76, wherein the method is effective in obtaining the progression-free survival at a rate of about 100.0% at a follow-up time of about 9 months.

79. The method of embodiment 76, wherein the method is effective in obtaining the progression-free survival at a rate of between about 63.2% and about 99.1% at a follow-up time of about 12 months, wherein optionally the method is effective in obtaining the progression-free survival at a rate of about 93.8% at a follow-up time of about 12 months.

80. The method of embodiment 76, wherein the method is effective in obtaining the progression-free survival at a rate of between about 63.2% and about 99.1% at a follow-up time of about 18 months, wherein optionally the method is effective in obtaining the progression-free survival at a rate of about 93.8% at a follow-up time of about 18 months.

81. The method of any one of embodiments 1-48, wherein the method is effective in obtaining an overall survival rate.

82. The method of embodiment 81, wherein the overall survival rate is about 100.0% at a follow-up time of about 6 months.

83. The method of embodiment 81, wherein the overall survival rate is about 100.0% at a follow-up time of about 9 months.

84. The method of embodiment 81, wherein the overall survival rate is between about 63.2% and about 99.1% at a follow-up time of about 12 months, wherein optionally of the overall survival rate is about 93.8% at a follow-up time of about 12 months.

85. The method of embodiment 81, wherein the overall survival rate is between about 63.2% and about 99.1% at a follow-up time of about 18 months, wherein optionally the overall survival rate is about 93.8% at a follow-up time of about 18 months.

86. The method of any one of embodiments 1-85, wherein the method further comprises treating the subject for an adverse event after the administering of the dose of the T cells.

87. The method of embodiment 86, wherein the method comprises administering a treatment to the subject to alleviate the adverse event.

88. The method of embodiment 86 or 87, wherein the adverse event comprises a treatment-emergent adverse event, wherein optionally the treatment-emergent adverse event is a serious treatment-emergent adverse event.

89. The method of embodiment 88, wherein the treatment-emergent adverse event occurs within the later of about 100 days at or after the administering of the dose of the T cells or about 30 days after last dose of lenalidomide.

90. The method of embodiment 88 or 89, wherein severity of the treatment-emergent adverse event is Grade 1, Grade 2, Grade 3, or Grade 4.

91. The method of embodiment 90, wherein the severity of the treatment-emergent adverse event is Grade 3 or 4, wherein optionally maximum severity of treatment-emergent adverse is Grade 4.

92. The method of embodiment 90, wherein maximum severity of the treatment-emergent adverse event is Grade 3, wherein optionally the maximum severity of the treatment-emergent adverse event of Grade 3 occurs at a rate of about 17.6%.

93. The method of embodiment 90, wherein maximum severity of the treatment-emergent adverse event is Grade 4, wherein optionally the maximum severity of the treatment-emergent adverse event of Grade 4 occurs at a rate of about 82.4%.

94. The method of any one of embodiments 88-93, wherein the treatment-emergent adverse event comprises a blood or lymphatic system disorder, an immune system disorder, a gastrointestinal disorder, an infection, an infestation, a musculoskeletal or connective tissue disorder, a respiratory, thoracic or mediastinal disorder, a general disorder, an administrate site condition, a metabolism or nutrition disorder, a nervous system disorder, a skin or subcutaneous tissue disorder, an eye disorder, a cardiac disorder, an ear or labyrinth disorder, or a vascular disorder, wherein optionally the treatment-emergent adverse event occurs at a rate of at least about 10%.

95. The method of any one of embodiments 88-93, wherein the treatment-emergent adverse event comprises neutropenia, lymphopenia, thrombocytopenia, leukopenia, anemia, febrile neutropenia, cytokine release syndrome, hypogammaglobulinaemia, diarrhoea, nausea, abdominal distension, abdominal pain, upper abdominal pain, constipation, dyspepsia, vomiting, increased aspartate aminotransferase, increased alanine aminotransferase, increased blood lactate dehydrogenase, decreased CD4 lymphocytes, increased gamma-glutamyltransferase, decreased serum ferritin, an upper respiratory tract infection, COVID-19, nasopharyngitis, pneumonia, respiratory syncytial virus infection, Rhinovirus infection, sinusitis, myalgia, back pain, muscle spasm, musculoskeletal stiffness, pain in extremity, cough, productive cough, nasal congestion, rhinorrhoea, epistaxis, oropharyngeal pain, fatigue, chill, malaise, pyrexia, hyperferritinaemia, hypoalbuminaemia, hypocalcaemia, hypomagnesaemia, hypophosphataemia, headache, dysarthria, hyperhidrosis, pruritus, or sinus tachycardia, wherein optionally the treatment-emergent adverse event occurs at a rate of at least about 10%.

96. The method of any one of embodiments 88-93, wherein the treatment-emergent adverse event is a serious treatment-emergent adverse event that occurs at a rate of about 58.8%, wherein optionally maximum severity of the treatment-emergent adverse event is Grade 3 or Grade 4, wherein further optionally the maximum severity of the treatment-emergent adverse event of Grade 3 or Grade 4 occurs at a rate of about 52.9%.

97. The method of embodiment 96, wherein the serious treatment-emergent adverse event comprises an infection, an infestation, a nervous system disorder, a blood or lymphatic system disorder, an immune system disorder, an eye disorder, a gastrointestinal disorder, a musculoskeletal or connective tissue disorder, or a vascular disorder, wherein optionally the serious treatment-emergent adverse event occurs at a rate of at least about 2%.

98. The method of embodiment 96, wherein the serious treatment-emergent adverse event comprises pneumonia, Rhinovirus infection, COVID-19 pneumonia, metapneumovirus infection, influenzal pneumonia, viral pneumonia, respiratory syncytial virus infection, sinusitis, streptococcal sepsis, facial nerve disorder, facial paralysis, peripheral motor neuropathy, febrile neutropenia, neutropenia, cytokine release syndrome, diplopia, diarrhoea, musculoskeletal pain, or haematoma, wherein optionally the serious treatment-emergent adverse event occurs at a rate of at least about 2%.

99. The method of any one of embodiments 88-93, wherein the treatment-emergent adverse event comprises cytokine release syndrome (CRS), wherein optionally the CRS occurs at a rate of about 82.4%, wherein optionally:
(1) time to first onset of the CRS ranges from about 6 days to about 11 days after the administering of the dose of the T cells to the subject, wherein further optionally the time to the first onset of the CRS is at a median of about 8.0 days or at a mean of about 8.1 days;
(2) time to recovery of the CRS ranges from about 1 day to about 5 days, wherein further optionally the time to recovery of the CRS is at a median time of about 2.5 days or at a mean time of about 2.6 days;
(3) duration of the CRS ranges from about 1 day to about 5 days or from about 2 days to about 3 days, wherein further optionally the duration of the CRS is at a median time of about 2.5 days or at a mean time of about 2.6 days; or
(4) duration of the CRS of less than about 7 days occurs at a rate of about 100%.

100. The method of embodiment 99, wherein the CRS occurs at maximum toxicity grade of Grade 1, Grade 2, Grade 3, or Grade 4, wherein optionally:
(1) the CRS occurs at the maximum toxicity grade of Grade 1 at a rate of about 76.5%; or
(2) the CRS occurs at the maximum toxicity grade of Grade 2 at a rate of about 5.9%.

101. The method of embodiment 99 or 100, wherein the treatment comprises an anti-IL-6 receptor, an IL-1 receptor antagonist, a corticosteroid, IV fluids, a vasopressor, oxygen, an analgesic, an anti-inflammatory drug, an antiinfective, an antiepileptic, caffeine, or prochlorperazine, or any combination thereof, wherein optionally:
(1) the anti-IL-6 receptor comprises tocilizumab;
(2) the IL-1 receptor antagonist comprises anakinra; or
(3) oxygen comprises blow-by, nasal cannula low flow at a flow rate of at most about 6 L/min, nasal cannula high flow at a flow rate of at least about 6 L/min, face mask, non-rebreather mask, venturi mask, or positive pressure, or any combination thereof.

102. The method of any one of embodiments 99-101, wherein the treatment is effective at achieving a recovery or resolution of the CRS, wherein optionally the treatment is effective at achieving a recovery or resolution of the CRS at a rate of about 100%.

103. The method of any one of embodiments 88-93, wherein the treatment-emergent adverse event comprises immune effector cell-associated neurotoxicity (ICANS), wherein optionally the ICANS occurs at a rate of about 5.9%, and wherein optionally:
(1) time to first onset of the ICANS is about 7 days after the administering of the dose of the T cells to the subject, wherein further optionally the time to the first onset of the ICANS is at a median of about 7 days or at a mean of about 7 days;
(2) time to recovery of the ICANS is about 1 day, wherein further optionally the time to recovery of the ICANS is at a median time of about 1 day or at a mean time of about 1 day;
(3) duration of the ICANS is about 1 day, wherein further optionally the duration of the ICANS is at a median time of about 1 day or at a mean time of about 1 day; or
(4) the ICANS occurs concurrently with cytokine release syndrome (CRS) at a rate of about 5.9%.

104. The method of embodiment 103, wherein the ICANS occurs at maximum toxicity grade of Grade 1, Grade 2, Grade 3, or Grade 4, wherein optionally the ICANS occurs at maximum toxicity grade of Grade 1 at a rate of about 5.9%.

105. The method of embodiment 103 or 104, wherein the treatment comprises an anti-IL-6 receptor, an IL-1 receptor antagonist, a corticosteroid, or ceftazidime, or any combination thereof, wherein optionally:
(1) the anti-IL-6 receptor comprises tocilizumab; or
(2) the IL-1 receptor antagonist comprises anakinra.

106. The method of any one of embodiments 103-105, wherein the ICANS is recovered, resolved, not recovered, not resolved, recovered with sequelae, resolved with sequelae, recovering, resolving, or unknown, wherein optionally the treatment is effective at achieving a recovery or resolution of the ICANS at a rate of about 100%.

107. The method of any one of embodiments 86-93, wherein the adverse event comprises neurotoxicity, wherein optionally the neurotoxicity occurs at a rate of about 35.3%, wherein optionally:
(1) time to first onset of the neurotoxicity ranges from about 16 days to about 27 days after the administering of the dose of the T cells to the subject, wherein further optionally the time to the first onset of the neurotoxicity is at a median of about 21.0 days or at a mean of about 20.8 days;
(2) time to recovery of the neurotoxicity ranges from about 29 days to about 443 days, wherein further optionally the time to recovery of the neurotoxicity is at a median time of about 70.0 days or at a mean time of about 153.0 days;
(3) duration of the neurotoxicity ranges from about 29 days to about 791 days, wherein further optionally the duration of the neurotoxicity is at a median time of about 111.0 days or at a mean time of about 254.7 days.

108. The method of embodiment 107, wherein the neurotoxicity is recovered, resolved, not recovered, or not resolved, wherein optionally the neurotoxicity is recovered or resolved at a rate of about 23.5%, and wherein optionally the neurotoxicity is not recovered or not resolved at a rate of about 11.8%.

109. The method of any one of embodiments 86-95, wherein the adverse event is an adverse event of special interest, wherein optionally:
(1) the adverse event of special interest occurs at a rate of about 82.4%, wherein further optionally the adverse event of special interest occurs at least at Grade 3 at a rate of about 23.5%; or
(2) the adverse event of special interest comprises cytokine release syndrome (CRS), CAR-T cell related neurotoxicity, second primary malignancy, or a movement and neurocognitive treatment-emergent adverse event, or any combination thereof.

110. The method of embodiment 109, wherein the adverse event of special interest is CRS, wherein optionally the CRS occurs at a rate of about 82.4%, wherein further optionally the CRS occurs at least at Grade 3 at a rate of about 0%.

111. The method of embodiment 109, wherein the adverse event of special interest is CAR-T cell related neurotoxicity, wherein optionally:
(1) the CAR-T cell related neurotoxicity occurs at a rate of about 35.3%, wherein further optionally the CAR-T cell related neurotoxicity occurs at least at Grade 3 at a rate of about 5.9%; or
(2) the CAR-T cell related neurotoxicity comprises immune effector cell-associated neurotoxicity (ICANS) or other neurotoxicity, wherein further optionally the ICANS occurs at a rate of about 5.9%, wherein further optionally the ICANS occurs at least at Grade 3 at a rate of about 0%, wherein further optionally the other neurotoxicity occurs at a rate of about 35.3%, and wherein further optionally the other neurotoxicity occurs at least at Grade 3 at a rate of about 5.9%.

112. The method of embodiment 109, wherein the adverse event of special interest is second primary malignancy, wherein optionally the second primary malignancy comprises myelodysplastic syndrome, wherein optionally the second primary malignancy occurs at a rate of about 5.9%, wherein further optionally the second primary malignancy occurs at least at Grade 3 at a rate of about 5.9%.

113. The method of embodiment 109, wherein the adverse event of special interest is a movement and neurocognitive treatment-emergent adverse event, wherein optionally the movement and neurocognitive treatment-emergent adverse event does not occur in the subject.

114. The method of any one of embodiments 86-95, wherein the adverse event comprises prolonged cytopenia, wherein optionally the prolonged cytopenia comprises thrombocytopenia, neutropenia, lymphopenia, or anemia, or any combination thereof, wherein further optionally:
(1) thrombocytopenia occurs at Grade 3 or 4 at a rate of about 29.4% after the administering of the dose of the T cells to the subject, wherein further optionally thrombocytopenia recovers to Grade 2 or less by about 30 days or about 60 days, and wherein further optionally thrombocytopenia reoccurs at Grade 3 or 4 at a rate of about 0%;
(2) neutropenia occurs at Grade 3 or 4 at a rate of about 88.2% after the administering of the dose of the T cells to the subject, wherein further optionally neutropenia recovers to Grade 2 or less by about 30 days or about 60 days, and wherein further optionally neutropenia reoccurs at Grade 3 or 4 at a rate of about 35.3%;
(3) lymphopenia occurs at Grade 3 or 4 at a rate of about 100% after the administering of the dose of the T cells to the subject, wherein further optionally lymphopenia recovers to Grade 2 or less by about 30 days or about 60 days, and wherein further optionally lymphopenia reoccurs at Grade 3 or 4 at a rate of about 11.8%; or
(4) anemia occurs at Grade 3 or 4 at a rate of about 5.9% after the administering of the dose of the T cells to the subject, wherein further optionally anemia recovers to Grade 2 or less by about 30 days or about 60 days, and wherein further optionally anemia reoccurs at Grade 3 or 4 at a rate of about 0%.

115. The method of any one of embodiments 88-95, wherein the treatment-emergent adverse event comprises a treatment-emergent infection, wherein optionally:
(1) the treatment-emergent infection occurs at a rate of about 70.6%, wherein further optionally the treatment-emergent infection occurs at Grade 3 or 4 at a rate of about 29.4%;
(2) the treatment-emergent infection comprises an infection, an infestation, a viral infectious disorder, a bacterial infectious disorder, or a fungal infectious disorder, or any combination thereof;
(3) the treatment-emergent infection comprises an upper respiratory tract infection, nasopharyngitis, pneumonia, sinusitis, acute sinusitis, bronchitis, infectious enterocolitis, gastroenteritis, pharyngitis, COVID-19, respiratory syncytial virus infection, Rhinovirus infection, COVID-19 pneumonia, herpes zoster, influenza, metapneumovirus infection, oral herpes, influenzal pneumonia, viral pneumonia, Campylobacter infection, Enterococcal infection, bacterial respiratory tract infection, Streptococcal sepsis, Aspergillus infection, Candida infection, fungal foot infection, or tongue fungal infection, or any combination thereof.

116. The method of any one of embodiments 1-115, wherein the first VHH domain comprises a CDR1 comprising the amino acid sequence of SEQ ID NO: 18, a CDR2 comprising the amino acid sequence of SEQ ID NO: 19, a CDR3 comprising the amino acid sequence of SEQ ID NO: 20, and the second VHH domain comprises a CDR1 comprising the amino acid sequence of SEQ ID NO: 21, a CDR2 comprising the amino acid sequence of SEQ ID NO: 22, and a CDR3 comprising the amino acid sequence of SEQ ID NO: 23, wherein optionally the first VHH domain comprises the amino acid sequence of SEQ ID NO: 2 and the second VHH domain comprises the amino acid sequence of SEQ ID NO: 4, wherein optionally the first VHH domain is at the N-terminus of the second VHH domain, or the first VHH domain is at the C-terminus of the second VHH domain, further wherein optionally:
(a) the first VHH domain is linked to the second VHH domain via a linker, wherein optionally the linker comprises the amino acid sequence of SEQ ID NO: 3;
(b) the transmembrane domain is derived from a molecule selected from the group consisting of CD8α, CD4, CD28, CD137, CD80, CD86, CD152 and PD1, wherein optionally the transmembrane domain is derived from CD8a, wherein optionally the transmembrane domain comprises the amino acid sequence of SEQ ID NO: 6;
(c) the intracellular signaling domain comprises a primary intracellular signaling domain of an immune effector cell, wherein optionally the primary intracellular signaling domain is derived from CD3ζ, wherein optionally the primary intracellular signaling domain comprises the amino acid sequence of SEQ ID NO: 8;
(d) the intracellular signaling domain comprises a co-stimulatory signaling domain, wherein optionally the co-stimulatory signaling domain is derived from a co-stimulatory molecule selected from the group consisting of CD27, CD28, CD137, OX40, CD30, CD40, CD3, LFA-1, ICOS, CD2, CD7, LIGHT, NKG2C, B7-H3, ligands of CD83 and any combination thereof, wherein optionally the co-stimulatory signaling domain comprises a cytoplasmic domain of CD137, wherein optionally the co-stimulatory signaling domain comprises the amino acid sequence of SEQ ID NO: 7;
(e) the CAR further comprises a hinge domain located between the C-terminus of the extracellular antigen binding domain and the N-terminus of the transmembrane domain, wherein optionally the hinge domain is derived from CD8α, wherein optionally the hinge domain comprises the amino acid sequence of SEQ ID NO: 5;
(f) the CAR further comprises a signal peptide located at the N-terminus of the polypeptide, wherein optionally the signal peptide is derived from CD8α comprising the amino acid sequence of SEQ ID NO: 1; or
(g) the CAR comprises the amino acid sequence of SEQ ID NO: 17.

117. The method of anyone of embodiments 1-116, wherein the dose of the T cells is formulated in a composition comprising dimethyl sulfoxide (DMSO), wherein optionally the DMSO is at a concentration of about 5%.

118. A method of treating a subject with multiple myeloma, wherein the subject has not achieved a complete response after receiving an initial therapy comprising (1) 4 to 8 cycles of an induction therapy, (2) a high-dose chemotherapy, and (3) an autologous stem cell transplantation (ASCT), the method comprising:
(1) administering to the subject a dose of T cells comprising a chimeric antigen receptor (CAR) comprising:
   (a) an extracellular antigen binding domain capable of specifically binding to an epitope of B-cell maturation antigen (BCMA), wherein the extracellular antigen binding domain comprises a first VHH domain comprising the amino acid sequence of SEQ ID NO: 2 and a second VHH domain comprising the amino acid sequence of SEQ ID NO: 4,
   (b) a transmembrane domain comprising the amino acid sequence of SEQ ID NO: 6 and
   (c) an intracellular signaling domain comprising the amino acid sequence of SEQ ID NO: 8, and
   (2) optionally administering to the subject a dose of an immunomodulatory drug (IMiD) after administering to the subject the dose of the T cells.

119. A method of treating a subject with multiple myeloma, wherein the subject has not achieved a complete response after receiving an initial therapy comprising (1) 4 to 8 cycles of an induction therapy, (2) a high-dose chemotherapy, and (3) an autologous stem cell transplantation (ASCT), the method comprising:
(1) administering to the subject a dose of T cells comprising a chimeric antigen receptor (CAR) comprising the amino acid sequence of SEQ ID NO: 17, and
(2) optionally administering to the subject a dose of an immunomodulatory drug (IMiD) after administering to the subject the dose of the T cells.

120. A dose of T cells comprising a chimeric antigen receptor (CAR) for use in treating a subject with multiple myeloma,
wherein the subject has not achieved a complete response after receiving an initial therapy comprising a stem cell transplantation, and
wherein the CAR comprises:
   (a) an extracellular antigen binding domain capable of specifically binding to an epitope of B-cell maturation antigen (BCMA), wherein the extracellular antigen binding domain comprises a first VHH domain and a second VHH domain, and wherein the first VHH domain comprising a CDR1, a CDR2, and a CDR3 as set forth in the VHH domain comprising the amino acid sequence of SEQ ID NO: 2, and the second VHH domain comprising a CDR1, a CDR2, and a CDR3 as set forth in the VHH domain comprising the amino acid sequence of SEQ ID NO: 4,
   (b) a transmembrane domain, and
   (c) an intracellular signaling domain.

121. A dose of T cells comprising a chimeric antigen receptor (CAR) for use in treating a subject with multiple myeloma,
wherein the subject has not achieved a complete response after receiving an initial therapy comprising (1) 4 to 8 cycles of an induction therapy, (2) a high-dose chemotherapy, and (3) an autologous stem cell transplantation (ASCT), and
wherein the CAR comprises:
   (a) an extracellular antigen binding domain capable of specifically binding to an epitope of B-cell maturation antigen (BCMA), wherein the extracellular antigen binding domain comprises a first VHH domain comprising the amino acid sequence of SEQ ID NO: 2 and a second VHH domain comprising the amino acid sequence of SEQ ID NO: 4,
   (b) a transmembrane domain comprising the amino acid sequence of SEQ ID NO: 6 and
   (c) an intracellular signaling domain comprising the amino acid sequence of SEQ ID NO: 8.

122. A dose of T cells comprising a chimeric antigen receptor (CAR) for use in treating a subject with multiple myeloma,
wherein the subject has not achieved a complete response after receiving an initial therapy comprising (1) 4 to 8 cycles of an induction therapy, (2) a high-dose chemotherapy, and (3) an autologous stem cell transplantation (ASCT), and
wherein the CAR comprises the amino acid sequence of SEQ ID NO: 17.

123. Use of a dose of T cells comprising a chimeric antigen receptor (CAR) for the manufacture of a medicament for treating a subject with multiple myeloma,
wherein the subject has not achieved a complete response after receiving an initial therapy comprising a stem cell transplantation, and
wherein the CAR comprises:
   (a) an extracellular antigen binding domain capable of specifically binding to an epitope of B-cell maturation antigen (BCMA), wherein the extracellular antigen binding domain comprises a first VHH domain and a second VHH domain, and wherein the first VHH domain comprising a CDR1, a CDR2, and a CDR3 as set forth in the VHH domain comprising the amino acid sequence of SEQ ID NO: 2, and the second VHH domain comprising a CDR1, a CDR2, and a CDR3 as set forth in the VHH domain comprising the amino acid sequence of SEQ ID NO: 4,
   (b) a transmembrane domain, and
   (c) an intracellular signaling domain.

124. Use of a dose of T cells comprising a chimeric antigen receptor (CAR) for the manufacture of a medicament for treating a subject with multiple myeloma,
wherein the subject has not achieved a complete response after receiving an initial therapy comprising (1) 4 to 8 cycles of an induction therapy, (2) a high-dose chemotherapy, and (3) an autologous stem cell transplantation (ASCT), and
wherein the CAR comprises:
   (a) an extracellular antigen binding domain capable of specifically binding to an epitope of B-cell maturation antigen (BCMA), wherein the extracellular antigen binding domain comprises a first VHH domain comprising the amino acid sequence of SEQ ID NO: 2 and a second VHH domain comprising the amino acid sequence of SEQ ID NO: 4,
   (b) a transmembrane domain comprising the amino acid sequence of SEQ ID NO: 6 and
   (c) an intracellular signaling domain comprising the amino acid sequence of SEQ ID NO: 8.

125. Use of a dose of T cells comprising a chimeric antigen receptor (CAR) for the manufacture of a medicament for treating a subject with multiple myeloma,
wherein the subject has not achieved a complete response after receiving an initial therapy comprising (1) 4 to 8 cycles of an induction therapy, (2) a high-dose chemotherapy, and (3) an autologous stem cell transplantation (ASCT), and
wherein the CAR comprises the amino acid sequence of SEQ ID NO: 17.

### 5. EXAMPLES

The following examples are provided to further describe some of the embodiments disclosed herein. The examples are intended to illustrate, not to limit, the disclosed embodiments.

### 5.1. Example 1: Ciltacabtagene Autoleucel

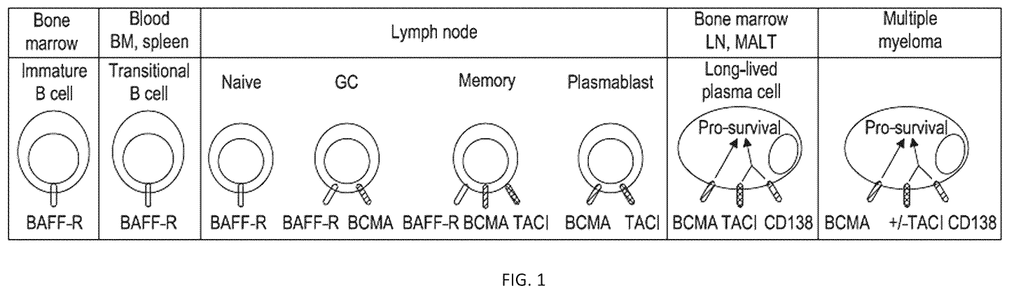

B cell maturation antigen (BCMA, also known as CD269 and TNFRSF17) is a 20 kilodalton, type III membrane protein that is part of the tumor necrosis receptor superfamily. BCMA is a cell surface antigen that is predominantly expressed in B-lineage cells at high levels. FIG. 1 shows the expression of BCMA on various immune-derived cells. Comparative studies have shown a lack of BCMA in most normal tissues and absence of expression on CD34-positive hematopoietic stem cells. BCMA binds 2 ligands that induce B cell proliferation, and plays a critical role in B cell maturation and subsequent differentiation into plasma cells. The selective expression and the biological importance for the proliferation and survival of myeloma cells makes BCMA a promising target for CAR-T based immunotherapy, ciltacabtagene autoleucel.

Ciltacabtagene autoleucel is an autologous chimeric antigen receptor T cell (CAR-T) therapy that targets BCMA. The ciltacabtagene autoleucel chimeric antigen receptor (CAR) comprises two B-cell maturation antigen (BCMA)-targeting VHH domains designed to confer avidity. A map of the construct is depicted in FIG. 2.

### 5.2. Example 2: Method of Treating Cohort D with Ciltacabtagene Autoleucel

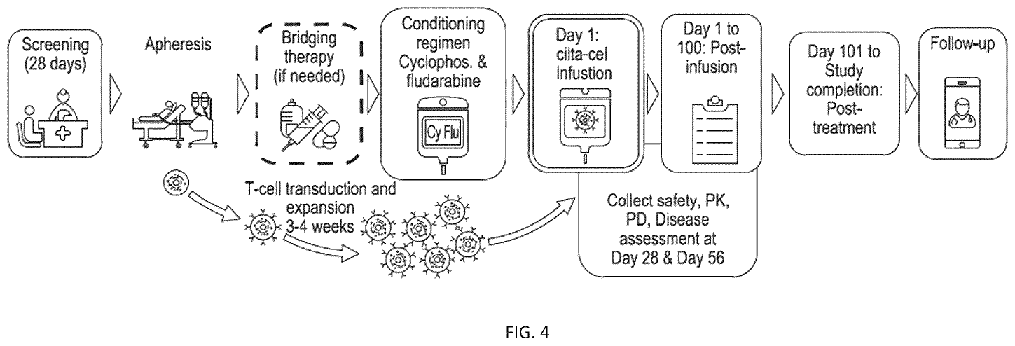

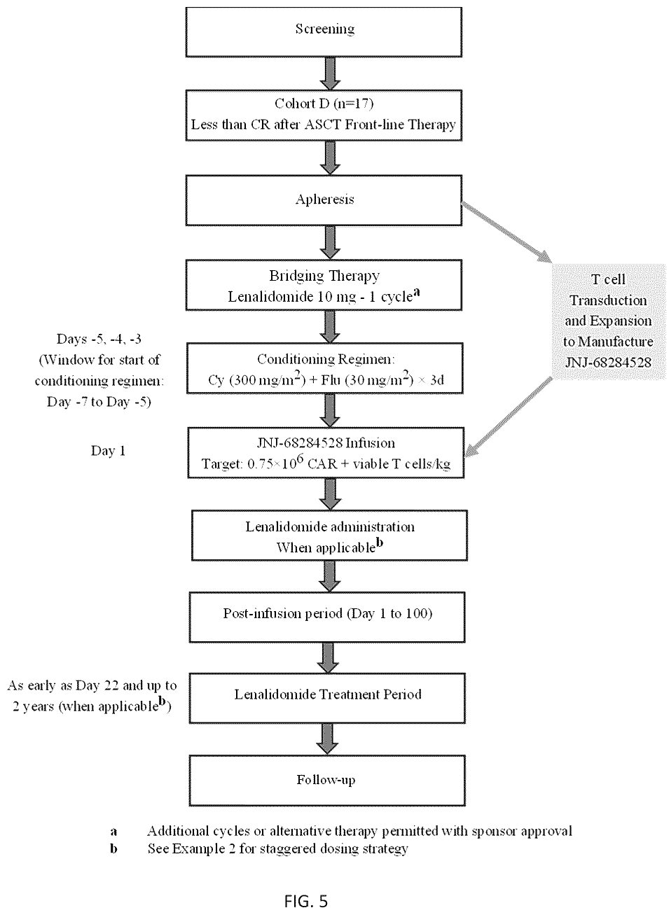

This is a Phase 2, multicohort, open-label, multicenter study to determine whether treatment with ciltacabtagene autoleucel (cilta-cel; JNJ-68284528), alone or with other treatment regimens, results in MRD negativity in adult subjects with multiple myeloma. Multiple patient populations of unmet medical need will be studied. The primary endpoint for all Cohorts will be overall MRD negative rate at a 10⁻⁵ threshold. A schematic overview of the study flow chart, which consists of a lymphodepleting regimen prior to cilta-cel infusion, is depicted in FIG. 4. Approximately 17 subjects were enrolled in Cohort D. Subjects were eligible for Cohort D if they have newly diagnosed multiple myeloma without complete response (best response <CR and ≥stable disease) after receiving an initial therapy comprising (1) 4 to 8 total cycles of an induction therapy, (2) a high-dose chemotherapy and (3) an ASCT with or without consolidation. A staggered dosing strategy applied to the first 10 subjects treated to allow for 28-day safety review of each subject individually as well as DMC reviews after each of the first two subgroups of 5 subjects received cilta-cel. The initial group of 5 subjects received cilta-cel only, with an observation period of at least 4 weeks between infusions for safety review of each subject followed by DMC review of these first 5 subjects. The subsequent group of 5 subjects received lenalidomide post-cilta-cel and continued with the same staggered dosing and review strategy. Based on DMC review, a decision was made to continue administering lenalidomide post-cilta-cel to the remaining subjects, without further dosing stagger. The protocol specified primary analysis of Cohort D is depicted in FIG. 5.

Treatment duration/Trial duration: Enrolled subjects underwent apheresis for cilta-cel manufacture. For all subjects, cilta-cel (JNJ-68284528) was generated from the subject's T cells selected from the apheresis product. Subjects for whom apheresis or manufacturing fails were allowed a second attempt at apheresis. Bridging therapy was allowed when clinically indicated, with the permission of the sponsor. After apheresis and prior to administration of cyclophosphamide and fludarabine (conditioning regimen prior to cilta-cel (JNJ-68284528) infusion) subjects received 1 or more cycles of lenalidomide at a dose of 10 mg per day upon adequate hematologic recovery from ASCT (absolute neutrophil count [ANC]≥1×10⁹/L and platelet count ≤75×10⁹/L)when minimum laboratory requirements are met. Alternative bridging therapy instead of, or in addition to, lenalidomide was permissible with sponsor approval. The purpose of bridging therapy is to reduce the myeloma burden prior to lymphodepletion chemotherapy and cilta-cel (JNJ-68284528) administration. After cilta-cel production and product release, subjects received a 3-day conditioning regimen of cyclophosphamide and fludarabine. After cilta-cel (JNJ-68284528) production and product release, all subjects received a conditioning regimen of IV cyclophosphamide 300 mg/m² and fludarabine 30 mg/m² daily for 3 days. Cilta-cel (JNJ-68284528) (e.g., 0.75×10⁶ CAR-positive viable T cells/kg) was administered 5 to 7 days after the start of the conditioning regimen. The conditioning regimen will lead to lymphodepletion and help promote CAR-T cell expansion in the subject.

Cilta-cel was administered 5 days to 7 days after the start of the conditioning regimen. The post-infusion period started after the completion of cilta-cel infusion on Day 1 and lasts until Day 100.

A strategy of staggered dosing with cilta-cel (JNJ-68284528) was applied to Cohort D. There was an observation period of at least 4 weeks between administration of cilta-cel (JNJ-68284528) to the first 5 subjects to allow for subject 28-day safety review prior to next subject dosing. In addition, the first 5 subjects did not receive lenalidomide after cilta-gel (JNJ-68284528) therapy to allow time for the Data Monitoring Committee to review safety data. After the first 5 subjects were dosed with cilta-cel (JNJ-68284528), the DMC convened to review safety and any other relevant data. Based on recommendation of the DMC, subsequent subjects in this cohort were eligible to receive lenalidomide after cilta-cel (JNJ-68284528). The same strategy of staggered dosing was also applied to the first 5 subjects receiving lenalidomide after cilta-cel (JNJ-68284528) (ie, the 6th through 10th subject enrolled in Cohort D). There was an observation period of at least 4 weeks between administration of cilta-cel (JNJ-68284528) (followed by lenalidomide) to these 5 subjects. The DMC reviewed safety and any other relevant data from the first 5 subjects who received cilta-cel (JNJ-68284528) followed by lenalidomide before a decision was made regarding the treatment plan for further subjects enrolled in Cohort D.

Subjects initiated lenalidomide maintenance therapy at a minimum of 21 days post cilta-cel (JNJ-68284528) infusion and after resolution of any cytokine release syndrome (CRS) or neurologic toxicities. Subjects continued to receive lenalidomide until confirmed PD, unacceptable toxicity, or for 2 years post cilta-cel (JNJ-68284528) infusion, whichever occurred first. The initial dose of lenalidomide post cilta-cel (JNJ-68284528) infusion depended on the level of hematologic recovery. Additionally, initiation of lenalidomide treatment was dependent on no additional safety concerns by investigator or sponsor.

The post-treatment period started on Day 101 and lasted until study completion, defined for Cohort D as 2.5 years after the last subject received his or her initial dose of cilta-cel.

Primary analysis set for efficacy and safety: The Modified Intent-To-Treat (mITT) analysis set consisted of subjects who received a cilta-cel infusion at the target dose and within range: 0.5-1.0×10⁶ CAR-positive viable T-cells/kg and was considered as the primary analysis set for all efficacy summaries. The All Treated analysis set consisted of subjects who received any dose of cilta-cel infusion and used as the primary analysis set for all safety summaries. All subjects within this cohort received cilta-cel infusion within the target dose range, hence the mITT and All Treated analysis sets were identical. Therefore, the All Treated analysis set was used for all efficacy and safety summaries of this report. The MRD based endpoint was also summarized using the MRD-evaluable analysis set, which consisted of subjects in the mITT analysis set who had at least one post-baseline MRD sample with positive or negative result from the central lab, or a local lab if central lab data was not available. The prespecified threshold for MRD was at 10⁻⁵ or greater sensitivity.

Primary endpoint: MRD negative rate at a 10⁻⁵ threshold as defined by the International Myeloma Working Group (IMWG) criteria using next generation sequencing (NGS) or next generation flow (NGF).

Major secondary endpoints: Overall Response Rate (ORR) (partial response or better); very good partial response (VGPR) or better rate (VGPR+complete response (CR)+stringent complete response (sCR)); clinical benefit rate (ORR+minimal response [MR]); duration of response (DOR); time to response (TTR); time to MRD negativity.

Expected effect size and planned sample size: No formal statistical hypothesis testing was performed. The sample size for Cohort D was selected to collect necessary data on preliminary efficacy and safety. The primary hypothesis is that cilta-cel will induce a deep response, measured by MRD negative rate.

Primary analysis for subjects in the Cohort D cohort occurred approximately 1 year after the last subject was dosed with cilta-cel.

Subjects were followed for efficacy, safety and survival after the clinical cutoff for the primary CSR. The cohort completion is defined as no later than 2.5 years after the last subject in Cohort D received cilta-cel, after which subjects continued to be followed or in a long-term follow-up study. (Per protocol, retreatment with cilta-cel is not permitted for Cohort D).

This report is generated from the protocol-specified primary analysis for the subjects who received cilta-cel in Cohort D.

The primary objective of this study was to evaluate the overall minimal residual disease (MRD) negative rate for subjects who receive cilta-cel.

### 5.3. Example 3: Evaluation of Efficacy of Method of Treating Cohort D with Ciltacabtagene Autoleucel

Using the IMWG-based response criteria summarized in Table 33, this study classified a response, in order from better to worse, as either a stringent complete response (sCR), a complete response (CR), a very good partial response (VGPR), a partial response (PR), a minimal response (MR), a stable disease or a progressive disease. Disease progression was consistently documented across clinical study sites. The tests performed to assess IMWG-based response criteria are as follows:
Myeloma Protein Measurements in Serum and Urine: Myeloma protein (M-protein) measurements were made using the following tests from blood and 24-hour urine samples: serum quantitative Ig, serum protein electrophoresis (SPEP), serum immunofixation electrophoresis, serum FLC assay (for subject in suspected CR/sCR and every disease assessment for subjects with serum FLC only disease), 24-hour urine M-protein quantitation by electrophoresis (UPEP), urine immunofixation electrophoresis, serum β2-microglobulin. Disease progression based on one of the laboratory tests alone were confirmed by at least 1 repeat investigation. Disease evaluations continued beyond relapse from CR until disease progression was confirmed. Serum and urine immunofixation and serum free light chain (FLC) assays were performed at screening and thereafter when a CR was suspected (when serum or 24-hour urine M-protein electrophoresis [by SPEP or UPEP] were 0 or non-quantifiable). For subjects with light chain multiple myeloma, serum and urine immunofixation tests were performed routinely.
Serum Calcium Corrected for Albumin: Blood samples for calculating serum calcium corrected for albumin were collected and analyzed until the development of confirmed disease progression; development of hypercalcemia (corrected serum calcium >11.5 mg/dL [>2.9 mmol/L]) may indicate disease progression or relapse if it is not attributable to any other cause. Calcium binds to albumin and only the unbound (free) calcium is biologically active; therefore, the serum calcium level must be adjusted for abnormal albumin levels (“corrected serum calcium”).
Bone Marrow Examination: Bone marrow aspirate or biopsy was performed for clinical assessments. Bone marrow aspirate was performed for biomarker evaluations. Clinical staging (morphology, cytogenetics, and immunohistochemistry or immunofluorescence or flow cytometry) was done. A portion of the bone marrow aspirate was immunophenotyped and monitor for BCMA, checkpoint ligand expression in CD138-positive multiple myeloma cells, and checkpoint expression on T cells. If feasible, bone marrow aspirate also was performed to confirm CR and sCR and at disease progression. Additionally, since minimal residual disease (MRD) negativity was being evaluated as a potential surrogate for PFS and OS in multiple myeloma treatment, MRD was monitored in subjects using next generation sequencing (NGS) on bone marrow aspirate DNA. Baseline bone marrow aspirates were used to define the myeloma clones, and post-treatment samples were used to evaluate MRD negativity. A fresh bone marrow aspirate was collected prior to the first dose of conditioning regimen (≤7 days).
Skeletal Survey: A skeletal survey (including skull, entire vertebral column, pelvis, chest, humeri, femora, and any other bones for which the investigator suspects involvement by disease) was performed during the screening phase and evaluated by either roentgenography (“X-rays”) or low-dose computed tomography (CT) scans without the use of IV contrast. If a CT scan was used, it was of diagnostic quality. Following cilta-cel infusion, and before disease progression was confirmed, X-rays or CT scans were performed locally, whenever clinically indicated based on symptoms, to document response or progression. Magnetic resonance imaging (MRI) was an acceptable method for evaluation of bone disease, and was included at discretion; however, it did not replace the skeletal survey. If a radionuclide bone scan was used at screening, in addition to the complete skeletal survey, then both methods were used to document disease status. These tests were performed at the same time. A radionuclide bone scan did not replace a complete skeletal survey. If a subject presented with disease progression manifested by symptoms of pain due to bone changes, then disease progression was documented by skeletal survey or other radiographs, depending on the symptoms that the subject experiences. If the diagnosis of disease progression was obvious by radiographic investigations, then no repeat confirmatory X-rays were thought necessary to perform. If changes were equivocal, then a repeat X-ray was performed in 1 to 3 weeks.
Documentation of Extramedullary Plasmacytomas: Sites of known extramedullary plasmacytomas were documented ≤14 days prior to the first dose of the conditioning regimen. Clinical examination or MRI were used to document extramedullary sites of disease. CT scan evaluations were considered an acceptable alternative if there was no contraindication to the use of IV contrast. Positron emission tomography scan or ultrasound tests were not acceptable to document the size of extramedullary plasmacytomas. However, PET/CT fusion scans were optionally used to document extramedullary plasmacytomas if the CT component of the PET/CT fusion scan was of sufficient diagnostic quality. Extramedullary plasmacytomas were assessed for all subjects with a history of plasmacytomas or if clinically indicated at ≤14 days prior to the first dose of the conditioning regimen, by clinical examination or radiologic imaging. Assessment of measurable sites of extramedullary disease were performed, measured, and evaluated locally every 4 weeks (for physical examination) for subjects with a history of plasmacytomas or as clinically indicated during treatment for other subjects until development of confirmed CR or confirmed disease progression. If assessment could only be performed radiologically, then evaluation of extramedullary plasmacytomas was done every 12 weeks. Irradiated or excised lesions were considered not measurable and were monitored only for disease progression. To qualify for VGPR or PR/minimal response (MR), the sum of products of the perpendicular diameters of the existing extramedullary plasmacytomas must have decreased by over 90% or at least 50%, respectively, and new plasmacytomas must not have developed. To qualify for disease progression, either the sum of products of the perpendicular diameters of the existing extramedullary plasmacytomas must have increased by at least 50%, or the longest diameter of previous lesion >1 cm in short axis must have increased at least 50%, or a new plasmacytoma must have developed. When not all existing extramedullary plasmacytomas were reported, but the sum of products of the perpendicular diameters of the reported plasmacytomas had increased by at least 50%, then the criterion for disease progression was met.

If it was determined that the study treatment interfered with the immunofixation assay, CR was defined as the disappearance of the original M-protein associated with multiple myeloma on immunofixation, and the determination of CR was not affected by unrelated M-proteins secondary to the study treatment.

Study endpoints, as assessed by an independent review committee (IRC), were as follows:
Overall response rate (ORR) was defined as the proportion of subjects who achieved a PR or better according to the IMWG criteria.
VGPR or better response rate (sCR+CR+VGPR) was defined as the proportion of subjects who achieve a VGPR or better response according to the IMWG criteria.
Duration of response (DOR) was calculated among responders (with a PR or better response) from the date of initial documentation of a response (PR or better) to the date of first documented evidence of progressive disease, as defined in the IMWG criteria. Relapse from CR by positive immunofixation or trace amount of M-protein was not considered as disease progression. Disease evaluations continued beyond relapse from CR until disease progression was confirmed.
Time to response (TTR) was defined as the time between date of the initial infusion of cilta-cel and the first efficacy evaluation at which the subject had met all criteria for PR or better.
Progression-free survival (PFS) was defined as the time from the date of the initial infusion of cilta-cel to the date of first documented disease progression, as defined in the IMWG criteria, or death due to any cause, whichever occurred first.
Overall survival (OS) was measured from the date of the initial infusion of cilta-cel to the date of the subject's death.

For ORR, the response rate and its 95% exact confidence interval (CI) was calculated based on binomial distribution, and the null hypothesis was rejected if the lower bound of the confidence interval exceeded 30%. Analysis of VGPR or better response rate, DOR, PFS, and OS was conducted at the same cutoff as the ORR. The distribution (median and Kaplan-Meier curves) of DOR was provided using Kaplan-Meier estimates. Similar analysis was performed for OS, PFS, and TTR.

### 5.4. Example 4: Evaluation of Safety of Method of Treating Cohort D with Ciltacabtagene Autoleucel

Adverse events were followed, reported and graded according to the National Cancer Institute Common Terminology Criteria for Adverse Events (NCI-CTCAE Version 5.0), with the exception of CRS and CAR-T cell-related neurotoxicity (e.g., ICANS). CRS was evaluated according to the ASTCT consensus grading, summarized in Table 34. At the first sign of CRS (such as fever), subjects were immediately hospitalized for evaluation. Tocilizumab intervention was discretionally used to treat subjects presenting symptoms of fever when other sources of fever had been eliminated. Tocilizumab was discretionally used for early treatment in subjects at high risk of severe CRS (for example, high baseline tumor burden, early fever onset, or persistent fever after 24 hours of symptomatic treatment). Other monoclonal antibodies targeting cytokines (for example, anti-IL1 and/or anti-TNFα) were optionally used, especially for cases of CRS which did not respond to tocilizumab.

CAR-T cell-related neurotoxicity (e.g., ICANS) was graded using the ASTCT consensus grading, summarized in Table 35. Additionally, all individual symptoms of CRS (e.g., fever, hypotension) and ICANS (e.g., depressed level of consciousness, seizures) were captured as individual adverse events and graded by CTCAE criteria. Neurotoxicity that was not temporarily associated with CRS, or any other neurologic adverse events that did not qualify as ICANS, were graded by CTCAE criteria. Any adverse event or serious adverse event not listed in the NCI CTCAE Version 5.0 was graded according to investigator clinical judgment by using the standard grades as follows:
Grade 1: Mild; asymptomatic or mild symptoms; clinical or diagnostic observations only; intervention not indicated.
Grade 2: Moderate; minimal, local or noninvasive intervention indicated; limiting age-appropriate instrumental activities of daily living.
Grade 3: Severe or medically significant but not immediately life-threatening; hospitalization or prolongation of hospitalization indicated; disabling; limiting self-care activities of daily living.
Grade 4: Life-threatening consequences; urgent intervention indicated.
Grade 5: Death related to adverse event.

### 5.5. Example 5: Efficacy and Safety of Ciltacabtagene Autoleucel in Patients with Newly Diagnosed Multiple Myeloma After An Initial Therapy (Cohort D)

### Subject and Treatment Information:

A total of 17 subjects were enrolled (underwent apheresis) into Cohort D between 4 Mar. 2020 and 27 Apr. 2022. Enrollment occurred across 12 sites in Belgium, France, Israel, Netherlands, Spain, USA. Of the 17 subjects enrolled, all 17 subjects received conditioning regimen and cilta-cel infusion within the target dose range. These 17 subjects constituted the All Treated analysis set, which was the basis for all demographics, baseline disease characteristics, safety and efficacy analyses presented below. At the clinical cutoff (5 Sep. 2023), the median duration of follow-up for the All Treated analysis set was 22.44 months. As per protocol design, the first 5 subjects received cilta-cel infusion only, the remaining 12 received cilta-cel infusion and lenalidomide maintenance therapy.

Median time since initial diagnosis of multiple myeloma to enrollment was 0.89 years. Median age was 54 years. All subjects had received an initial therapy comprising an ASCT, one subject received tandem ASCT.

### Primary Efficacy Endpoint:

The MRD negative rate (at 10-5) in the All Treated analysis set (N=17) was 70.6% (95% CI: 44%-89.7%). The median time to MRD negativity was 1.33 months (range: 0.9-6.1).

In the All Treated Analysis Set, all 17 subjects had a baseline MRD assessment, of which, 8 subjects achieved successful calibration. The remaining 9 subjects did not have successful calibration in that 8 subjects had clone identification failure and 1 subject had unsuccessful assay run via central lab-performed NGS. Among the 9 subjects without clone identified by NGS central, 4, 2 and 1 subjects had evaluable MRD by central NGF, local NGS and local NGF, respectively. Thus, 15 subjects constituted the MRD-evaluable analysis set. In the MRD-evaluable analysis set (N=15), the MRD negative rate (at 10-5) was 80% (95% CI: 51.9%-95.7%).

### Major Secondary Efficacy Endpoints:

Based on the patient population and largely expected per inclusion criteria, 13 subjects were categorized as Not Evaluable for IMWG baseline measurable disease type, therefore could only be assessed for CR, sCR or PD.

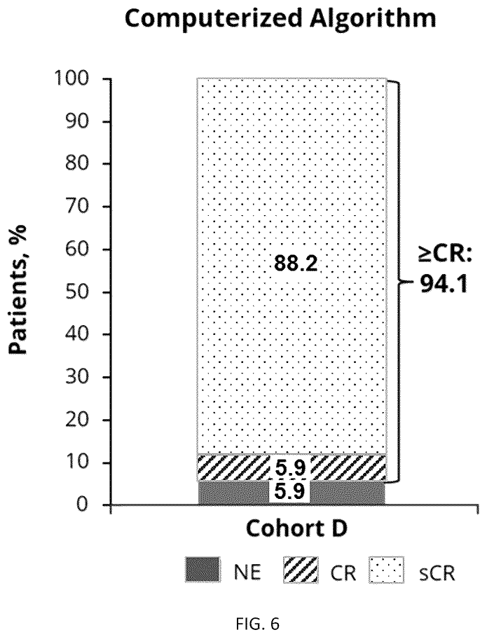

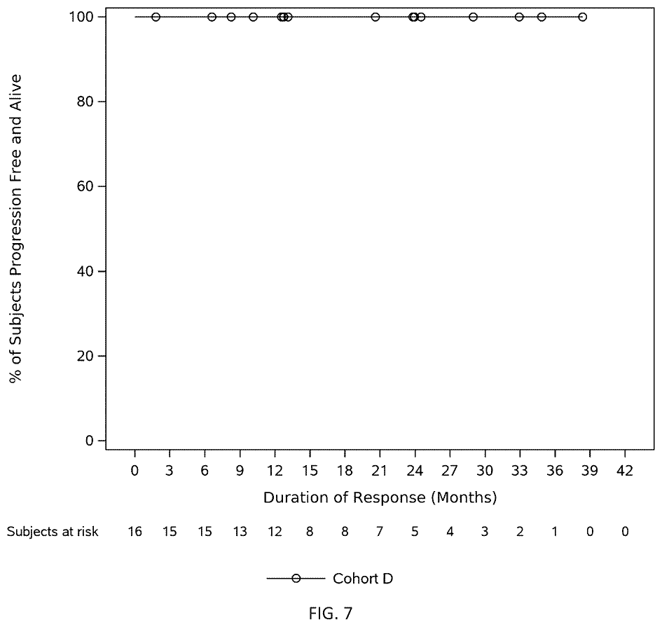

Overall response and DOR: In the All Treated Analysis Set, 16 subjects (94.1%, 95% CI: 71.3%-99.9%) achieved CR or better based on computerized algorithm as defined by the IMWG response criteria (FIG. 6). Median DOR was not reached; the probability of the responders remaining in response at 12 months was 100% (95% CI: 100.0%-100.0%), as depicted in FIG. 7. TTR: median time to first response (PR or better) and to best response were 1.30 and 1.89 months, respectively. PFS: median was not reached; 12-month PFS rate (95% CI) was 93.8% (63.2%-99.1%). OS: median was not reached; 12-month survival rate (95% CI) was 93.8% (63.2%-99.1%).

### Exposure:

In the All Treated analysis set, the median (range) of cilta-cel dose formulated and dose administered were 0.70 (0.60-0.80) and 0.68 (0.56-0.84)×10⁶ CAR-positive viable T cells/kg, respectively. Median (range) time since initial apheresis to cilta-cel infusion was 85.0 (49-131) days. Median (range) time since receipt of apheresis material at Manufacturing Facility up to, and inclusive of the day on which CAR-T product is released was 38.0 (27-68) days. One subject received product via exceptional release, this subject was administered two low dose infusion bags of cilta-cel at 0.4×10⁶ CAR-positive viable T cells/kg each (total dose 0.8×10⁶ CAR-positive viable T cells/kg) to meet the target dose range.

Twelve subjects started lenalidomide maintenance therapy at a median (range) of 51 days (21-214) after cilta-cel infusion, and continued the lenalidomide maintenance therapy for a median (range) duration of 426 days (70-716) days. Median (range) number of cycles received was 15 (3-26) with an overall median (range) relative dose intensity of 93.4% (67.9%-100.0%) across all cycles received.

### Safety:

In the All Treated analysis set, all 17 (100%) subjects experienced at least one treatment-emergent adverse event (TEAE) defined as any AE that occurs within 100 days at or after cilta-cel infusion or 30 days after last dose of lenalidomide maintenance (whichever is later), or any AE that is considered related to the cilta-cel or lenalidomide maintenance regardless of the start date of the event. The most common (at least 20%) TEAEs were: anemia, cough, COVID-19, cytokine release syndrome, diarrhea, fatigue, hypogammaglobulinaemia, leukopenia, lymphopenia, myalgia, nasopharyngitis, nausea, neutropenia, productive cough, thrombocytopenia, upper respiratory tract infection.

Seventeen (100.0%) subjects experienced at least one Grade 3 or higher TEAE. No subjects experienced Grade 5 TEAE.

Ten (58.8%) subjects experienced treatment-emergent SAEs, of which 9 subjects received lenalidomide maintenance therapy. The treatment-emergent SAEs were: COVID-19 pneumonia, cytokine release syndrome, diarrhea, diplopia, facial nerve disorder, facial paralysis, febrile neutropenia, hematoma, metapneumovirus infection, musculoskeletal pain, neutropenia, peripheral motor neuropathy, pneumonia, pneumonia influenza, pneumonia viral, respiratory syncytial virus infection, rhinovirus infection, sinusitis, streptococcal sepsis.

One death occurred during the study; this subject died 332 days after cilta-cel infusion due to progressive disease.

### Adverse Events of Special Interest:

CRS: All-grade CRS was reported for 14 (82.4%) subjects, with all subjects experiencing a Grade 1 or 2 event, evaluated by the ASTCT consensus grading system. Median time from cilta-cel infusion to CRS onset was 8 days (range: 6-11). All events of CRS had recovered with median time to recovery of 2.5 days (range: 1-5).

### Neurotoxicity was Reported for 6 (35.3%) Subjects:

(1) Immune Effector Cell-Associated Neurotoxicity (ICANS): All-grade ICANS was reported for 1 (5.9%) subject. The subject had a grade 1 event, evaluated by the ASTCT consensus grading system. with onset on Day 7 and recovered the same day.
(2) Other CAR-T neurotoxicities (not reported as ICANS; determined by investigator to be related to CAR-T cell therapy and onset after recovery from CRS and ICANS): All-grade other CAR-T neurotoxicities were reported for 6 (35.3%) subjects, with 1 (5.9%), 4 (23.5%) and 1 (5.9%) subjects experiencing Grade 1, 2 and 3 events, respectively. Median time from cilta-cel infusion to first onset of other neurotoxicity was 21 days (range: 16-27). Four events of other CAR-T neurotoxicities had recovered and the median time to recovery was 36 days (range: 12-443). Two subjects, one with grade 1 facial nerve palsy and the other with grade 1 paresthesia, have not recovered after 288 days and 473 days from their respective events at the time of CCO.
(3) No Movement and Neurocognitive TEAEs (MNTs) were reported.

One second primary malignancy was reported. The subject experienced grade 3 myelodysplastic syndrome with onset on Day 353, which was considered not related to cilta-cel as per investigator assessment.

### Other Safety Observations:

Cytopenias: 17 (100.0%), 15 (88.2%), 5 (29.4%) and 1 (5.9%) subjects had Grade 3 or 4 lymphopenia, neutropenia thrombocytopenia and anemia, respectively, in the first 100 days after cilta-cel infusion. The initial Grade 3 or 4 event for all but 7 (41.2%), 1 (5.9%), 1 (5.9%) and 0 (0.0%) subjects recovered to Grade 2 or less by Day 30 for lymphopenia, neutropenia, thrombocytopenia and anemia, respectively. Five (29.4%), 1 (5.9%), 1 (5.9%) and 0 (0.0%) subjects had their initial Grade 3 or 4 event not recovered to Grade 2 or lower by Day 60 for lymphopenia, neutropenia, thrombocytopenia and anemia, respectively. After Day 60 and following initial recovery, Grade 3/4 cytopenia reoccurred in 2 (11.8%), 6 (35.3%), 0 (0.0%) and 0 (0.0%) subjects for lymphopenia, neutropenia, thrombocytopenia and anemia, respectively.

Infections: 12 (70.6%) subjects had an infection TEAE, of whom 5 (29.4%) subjects had Grade 3 or 4 events.

### Conclusion:

Data from this cohort establishes a positive benefit/risk profile for cilta-cel in the treatment of patients who did not achieve a CR after receiving an initial therapy comprising (1) 4 to 8 total cycles of an induction therapy, (2) a high-dose chemotherapy and (3) an ASCT with or without consolidation. In summary:

In this patient population, single-agent and one-time infusion of cilta-cel, with or without lenalidomide maintenance demonstrated deep response with MRD negative rate (at 10−5) of 70.6% in the All Treated analysis set (N=17) and 80% in the MRD-evaluable analysis set (N=15), as well as substantial clinical activity, with an ORR of 94.1% and rapid onset of response in approximately 1 month.

The deep and durable clinical response induced by cilta-cel with or without lenalidomide maintenance were demonstrated by a CR or better rate of 94.1%, and median DOR was not reached. The probability of the responders remaining in response at 12 months was 100% (95% CI: 100.0%-100.0%).

With a median duration of follow-up of 22.44 months, median PFS was not reached. The 12-month PFS rate (95% CI) was 93.8% (63.2%-99.1%). Median OS was not reached. The 12-month survival rate (95% CI) was 93.8% (63.2%-99.1%).

In this population, cilta-cel has a safety profile consistent with the mechanism of action of CAR-T therapy similarly demonstrated in the CARTITUDE-1 study. CAR-T cell-related adverse events of CRS were common (82.4%) but all were low grade. Neurological toxicities reported included ICANS (5.9%) and other neurotoxicities (35.3%). An Urgent Safety Measure (USM) was issued on 11 Mar. 2020 to mitigate the risk of MNTs as identified in the CARTITUDE-1 study. All 17 subjects treated in this Cohort were dosed after implementation of this USM. No MNTs were reported. Grade 3 or 4 cytopenias were common in the post-infusion period, including lymphopenia (100.0%), neutropenia (88.2%), thrombocytopenia (29.4%), anemia (5.9%); Around 70% or more of these cases recovered by Day 60, with a majority recovered by Day 30. Grade 3 or higher infections were reported for 29.4% of subjects.

The teachings of all patents, published applications, and references cited herein are incorporated by reference in their entirety.

While example embodiments have been particularly shown and described, it will be understood by those skilled in the art that various changes in form and details may be made therein without departing from the scope of the embodiments encompassed by the appended claims.

TABLE 1
Summary of Study Duration of Follow-up; Cohort D
All Treated Analysis Set (Study 68284528MMY2003)

| | Total |
|---|---|
| Analysis set: all treated | 17 |
| Duration of follow-up | |
| (months) | |
| N | 17 |
| Mean (SD) | 22.04 (9.956) |
| Median ^{a} | 22.44 |
| Range | (4.7; 39.3) |
| Subjects with follow-up ≥3 | 17 |
| months | |

^{a} Based on Kaplan-Meier product limit estimate.
Note:
Median study duration of follow-up was 15.18 (range: 4.7, 29.9) months for the 12 subjects in Cohort D who received lenalidomide after cilta-cel infusion.
Note:
Duration of follow-up is in reference to the date of the cilta-cel (JNJ-68284528) infusion (Day 1).

TABLE 2
Summary of Subject Study Disposition; Cohort D All
Enrolled Analysis Set (Study 68284528MMY2003)

| | Total | |
|---|---|---|
| Analysis set: all enrolled | 17 | |
| Discontinued the study | 3 | (17.6%) |
| After apheresis and prior to the start | 0 | |
| of conditioning regimen | | |
| After the start of conditioning regimen | 0 | |
| and prior to JNJ-68284528 infusion | | |
| After JNJ-68284528 infusion | 3 | (17.6%) |
| Reason for discontinuation | | |
| Withdrawal by subject | 1 | (5.9%) |
| Lost to follow-up | 1 | (5.9%) |
| Death | 1 | (5.9%) |
| Ongoing in follow-up ^{a} | 14 | (82.4%) |

^{a} Subjects on study refers to after apheresis.
Note:
Percentages are calculated with the number of subjects in the all enrolled analysis set as denominator.

TABLE 3
Summary of Subject Treatment Overview; Cohort D
All Enrolled Analysis Set (Study 68284528MMY2003)

| | Total | |
|---|---|---|
| Analysis set: all enrolled | 17 | |
| Subject who underwent apheresis | 17 | (100.0%) |
| Subjects who received conditioning | 17 | (100.0%) |
| regimen | | |
| Subjects who received JNJ-68284528 | 17 | (100.0%) |
| infusion | | |
| Subjects received conditioning regimen | 0 | |
| but did not receive JNJ-68284528 | | |
| infusion | | |
| Subjects who received JNJ-68284528 | | |
| infusion | | |
| without Lenalidomide maintenance | 5 | (29.4%) |
| with Lenalidomide maintenance | 12 | (70.6%) |

Note:
Percentages are calculated with the number of subjects in the all enrolled analysis set as denominator.

TABLE 4
Summary of Baseline Disease Characteristics; Cohort
D All Treated Analysis Set (Study 68284528MMY2003)

| | Total | |
|---|---|---|
| Analysis set: all treated | 17 | |
| Type of myeloma by immunofixation, | | |
| n (%) | | |
| N | 17 | |
| IgG | 11 | (64.7%) |
| IgA | 2 | (11.8%) |
| IgM | 0 | |
| IgD | 0 | |
| IgE | 0 | |
| Light chain | 2 | (11.8%) |
| Kappa | 2 | (11.8%) |
| Lambda | 0 | |
| Biclonal | 0 | |
| Negative immunofixation | 2 | (11.8%) |
| Type of measurable disease, n (%) | | |
| N | 17 | |
| Serum only | 2 | (11.8%) |
| Serum and urine | 0 | |
| Urine only | 0 | |
| Serum FLC only | 2 | (11.8%) |
| Not evaluable | 13 | (76.5%) |
| ISS staging at study baseline ^{a}, n (%) | | |
| N | 17 | |
| I | 17 | (100.0%) |
| II | 0 | |
| III | 0 | |
| Time since initial MM diagnosis to | | |
| enrollment, years | | |
| N | 17 | |
| Mean (SD) | 0.92 | (0.203) |
| Median | 0.89 | |
| Range | (0.6; 1.4) | |
| Number of lytic bone lesions | | |
| N | 17 | |
| None | 6 | (35.3%) |
| 1-3 | 2 | (11.8%) |
| 4-10 | 4 | (23.5%) |
| More than 10 | 5 | (29.4%) |
| Presence of extramedullary plasmacytomas | | |
| N | 17 | |
| Yes | 0 | |
| No | 17 | (100.0%) |
| Presence of evaluable bone marrow | | |
| assessment | | |
| N | 17 | |
| Yes | 17 | (100.0%) |
| No | 0 | |
| % Plasma cells, bone marrow | | |
| biopsy/aspirate ^{b} | | |
| N | 17 | |
| ≤30 | 17 | (100.0%) |
| >30-<60 | 0 | |
| ≥60 | 0 | |
| % Plasma cells, bone marrow biopsy | | |
| N | 14 | |
| ≤30 | 14 | (100.0%) |
| >30-<60 | 0 | |
| ≥60 | 0 | |
| % Plasma cells, bone marrow aspirate | | |
| N | 17 | |
| ≤30 | 17 | (100.0%) |
| >30-<60 | 0 | |
| ≥60 | 0 | |
| Bone marrow cellularity by biopsy | | |
| N | 11 | |
| Hypercellular | 0 | |
| Normocellular | 5 | (45.5%) |
| Hypocellular | 5 | (45.5%) |
| Indeterminate | 1 | (9.1%) |
| Cytogenetic risk at study baseline ^{c} | | |
| N | 17 | |
| Standard Risk | 15 | (88.2%) |
| High Risk | 1 | (5.9%) |
| del 17p | 1 | (5.9%) |
| t(4; 14) | 0 | |
| t(14; 16) | 0 | |
| Unknown | 1 | (5.9%) |
| Tumor BCMA expression at study | | |
| baseline (%) | | |
| N | 2 | |
| Mean (SD) | 86.8 | (1.63) |
| Median | 86.8 | |
| Range | (86; 88) | |
| ≥50% | 2 | (100.0%) |

Keys: FLC = free light chain; ISS = International Staging System; MM = multiple myeloma.
^{a} ISS staging is derived based on serum β2-microglobulin (via central assessment prior to conditioning therapy) and albumin.
^{b} Maximum value from bone marrow biopsy and bone marrow aspirate is selected if both the results are available.
^{c} Cytogenetic risk abnormalities are based on central fluorescence in situ hybridization (FISH) testing, or local FISH testing and karyotype testing if central FISH not available.
Note:
Negative immunofixation: 2 (N05BE10004005: screening BMa pc 10%, N05US10015010 screening BMb pc 15%).

TABLE 5
Summary of Demographics and Baseline Characteristics; Cohort
D All Treated Analysis Set (Study 68284528MMY2003)

| | Total | |
|---|---|---|
| Analysis set: all treated | 17 | |
| Age, years | | |
| N | 17 | |
| Category, n (%) | | |
| <65 | 14 | (82.4%) |
| 65-75 | 3 | (17.6%) |
| Mean (SD) | 53.4 | (10.82) |
| Median | 54.0 | |
| Range | (37; 69) | |
| Sex | | |
| N | 17 | |
| Male | 14 | (82.4%) |
| Female | 3 | (17.6%) |
| Race | | |
| N | 17 | |
| Black or African American | 1 | (5.9%) |
| White | 14 | (82.4%) |
| Not reported | 2 | (11.8%) |
| Ethnicity | | |
| N | 17 | |
| Hispanic or Latino | 1 | (5.9%) |
| Not Hispanic or Latino | 14 | (82.4%) |
| Not reported | 2 | (11.8%) |
| Weight, kg | | |
| N | 17 | |
| Mean (SD) | 84.2 | (23.00) |
| Median | 79.0 | |
| Range | (47; 139) | |
| Height, cm | | |
| N | 17 | |
| Mean (SD) | 171.8 | (11.92) |
| Median | 170.0 | |
| Range | (145; 190) | |
| Body surface area (BSA), m² | | |
| N | 17 | |
| Mean (SD) | 1.99 | (0.307) |
| Median | 2.01 | |
| Range | (1.4; 2.6) | |
| ECOG score at screening | | |
| N | 17 | |
| 0 | 13 | (76.5%) |
| 1 | 4 | (23.5%) |
| ECOG score prior to conditioning regimen | | |
| N | 17 | |
| 0 | 13 | (76.5%) |
| 1 | 4 | (23.5%) |
| ECOG score prior to JNJ-68284528 | | |
| infusion^{a} | | |
| N | 17 | |
| 0 | 14 | (82.4%) |
| 1 | 2 | (11.8%) |
| 2 | 1 | (5.9%) |

Key: ECOG = Eastern Cooperative Oncology Group
^{a}The last non-missing ECOG score on or prior to date of JNJ-68284528 infusion is used. All patients met the inclusion criteria of ECOG score of 0 or 1 during screening.

TABLE 6
Summary of Prior Therapies for Multiple Myeloma; Cohort
D All Treated Analysis Set (Study 68284528MMY2003)

| | Total | |
|---|---|---|
| Analysis set: all treated | 17 | |
| Number of lines of prior therapies | | |
| for multiple myeloma | | |
| N | 17 | |
| Category, n (%) | | |
| 1 | 17 | (100.0%) |
| 2 | 0 | |
| 3 | 0 | |
| Mean (SD) | 1.0 | (0.00) |
| Median | 1.0 | |
| Range | (1; 1) | |
| Prior transplantation | 17 | (100.0%) |
| Autologous | 17 | (100.0%) |
| 1 time | 16 | (94.1%) |
| 2 times | 1 | (5.9%) |
| 3 times | 0 | |
| Allogeneic | 0 | |
| Prior radiotherapy | 4 | (23.5%) |
| Prior cancer-related surgery/procedure | 3 | (17.6%) |
| Prior PI | 17 | (100.0%) |
| Bortezomib | 16 | (94.1%) |
| Carfilzomib | 2 | (11.8%) |
| Ixazomib | 0 | |
| Prior IMiD | 17 | (100.0%) |
| Lenalidomide | 11 | (64.7%) |
| Pomalidomide | 0 | |
| Thalidomide | 6 | (35.3%) |
| Prior PI and Prior IMiD | 17 | (100.0%) |
| Prior corticosteroids | 17 | (100.0%) |
| Dexamethasone | 17 | (100.0%) |
| Prednisone | 0 | |
| Prior alkylating agents | 17 | (100.0%) |
| Prior anthracyclines | 0 | |
| Prior anti-CD38 antibodies | 3 | (17.6%) |
| Daratumumab | 3 | (17.6%) |
| Isatuximab | 0 | |
| Prior Elotuzumab | 0 | |
| Prior Panobinostat | 0 | |
| Prior PI + IMiD + ALKY | 17 | (100.0%) |
| Prior PI + IMiD + anti-CD38 antibodies | 3 | (17.6%) |
| Prior PI + IMiD + anti-CD38 antibodies + ALKY | 3 | (17.6%) |

| | |
|---|---|
| Prior penta-exposed (at least 2 PIs + at | 0 |
| least 2 IMiDs + 1 anti-CD38 antibodies) | |

Keys: ALKY = alkylating agents; IMiD = Immunomodulatory agent; PI = proteasome inhibitor.
Note:
One patient received tandem autologous transplantation.
Note:
Percentages are calculated with the number of subjects in the all treated analysis set as denominator.

TABLE 7
Summary of Refractory Status to Prior Multiple Myeloma Therapy;
Cohort D All Treated Analysis Set (Study 68284528MMY2003)

| | Total |
|---|---|
| Analysis set: all treated | 17 |
| Refractory at any point to prior therapy | 0 |
| Refractory Status | |
| PI + IMiD + anti-CD38 antibody | 0 |
| Any PI | 0 |
| Any IMiD | 0 |
| Any anti-CD38 antibody | 0 |
| At least 2 PIs + at least 2 IMiDs + 1 anti- | 0 |
| CD38 antibody | |
| Refractory to last line of prior therapy | 0 |
| Refractory to | |
| Bortezomib | 0 |
| Carfilzomib | 0 |
| Ixazomib | 0 |
| Lenalidomide | 0 |
| Pomalidomide | 0 |
| Thalidomide | 0 |
| Daratumumab | 0 |
| Isatuximab | 0 |
| Elotuzumab | 0 |

Keys: IMiD = Immunomodulatory agent; PI = proteasome inhibitor.
Note:
Refractory to a medication refers to any line that contained that medication.
Note:
Percentages are calculated with the number of subjects in the all treated analysis set as denominator.

TABLE 8
Listing of Disease Response Assessment by Computerized Algorithm;
Cohort D All Analysis Set (Study 68284528MMY2003)

| | First Response | Best Confirmed | |
|---|---|---|---|
| | and Date of | Response and Date | Date of PD |
| | First Response | of Best Confirmed | (Study Day^{a})and |
| Subject ID | (Study Day^{a}) | Response (Study Day^{a}) | Reason |
| N05BE10002004 | CR/2020 Nov. 24(30) | sCR/2020 Dec. 22(58) | |
| N05BE10004001 | CR/2020 Sep. 14(78) | sCR/2020 Oct. 08(102) | |
| N05BE10004005 | sCR/2021 Apr. 06(30) | sCR/2021 Apr. 06(30) | |
| N05BE10004006 | sCR/2021 Nov. 22(57) | sCR/2021 Nov. 22(57) | |
| N05ES10002008 | sCR/2021 Aug. 19(246) | sCR/2021 Aug. 19(246) | |
| N05ES10005011 | CR/2022 Jul. 04(29) | sCR/2022 Jul. 27(52) | |
| N05FR10001002 | CR/2023 Jul. 13(379) | CR/2023 Jul. 13(379) | |
| N05IL10001001 | sCR/2020 Jun. 10(30) | sCR/2020 Jun. 10(30) | |
| N05IL10001012 | VGPR/2021 Aug. 29(28) | sCR/2021 Sep. 29(59) | |
| N05IL10001014 | PR/2022 Jul. 03(28) | sCR/2023 Jun. 21(381) | |
| N05IL10001017 | CR/2022 Sep. 18(49) | sCR/2022 Sep. 20(51) | |
| N05NL10002001 | sCR/2021 Aug. 10(56) | sCR/2021 Aug. 10(56) | |
| N05US10002006 | CR/2022 Jul. 19(30) | sCR/2022 Jul. 20(31) | |
| N05US10004008 | sCR/2022 Jul. 13(240) | sCR/2022 Jul. 13(240) | |
| N05US10010001 | sCR/2021 Jan. 28(157) | sCR/2021 Jan. 28(157) | |
| N05US10015010 | sCR/2022 Jun. 28(28) | sCR/2022 Jun. 28(28) | |
| N05US10027002 * | | NE | |

Keys: CR = complete response; MR = minimal response; NE = not evaluable; PD = progressive disease; PR = partial response; SD = stable disease; sCR = stringent response; VGPR = very good partial response.
* Patient had NE at baseline and later withdrew consent prior to reaching CR/sCR.
^{a}Based on initial JNJ-68284528 infusion date (Day 1) as reference.

TABLE 9
Summary of Overall MRD Negativity Rate at 10⁻⁵ in Bone Marrow;
Cohort D All Treated Analysis Set (Study 68284528MMY2003)

| | Total |
|---|---|
| Analysis set: all treated | 17 |
| MRD negativity rate (10⁻⁵) | 12 (70.6%) |
| 95% CI^{a} of MRD negative rate | (44.0%, 89.7%) |

Key: CI = confidence interval.
^{a}Exact 95% confidence interval.
Note:
MRD status result based on next-generation sequencing (NGS) or next-generation flow (NGF).
Note:
MRD results were based on either central or local lab assessments.

TABLE 10
Summary of Overall MRD Negativity Rate at 10⁻⁵ in Bone
Marrow; Cohort D Subjects with Evaluable Sample at 10 −
5 in All Treated Analysis Set (Study 68284528MMY2003)

| | Total |
|---|---|
| Analysis set: subjects with evaluable | 15 |
| sample at 10⁻⁵ in all treated | |
| MRD negativity rate (10⁻⁵) | 12 (80.0%) |
| 95% CI^{a} of MRD negative rate | (51.9%, 95.7%) |

Keys: CI = confidence interval; MRD = minimal residual disease.
^{a}Exact 95% confidence interval.
Note:
Evaluable samples are those that pass calibration and QC, and include sufficient cells for evaluation at the respective testing threshold.
Note:
MRD status result based on next-generation sequencing (NGS) or next-generation flow (NGF).
Note:
MRD results were based on either central or local lab assessments.

TABLE 11
Summary of Time to MRD Negativity at 10⁻⁵ in Bone Marrow;
Cohort D All Treated Analysis Set (Study 68284528MMY2003)

| | Total |
|---|---|
| Analysis set: all treated | 17 |
| Time to MRD negativity (months) | |
| N | 12 |
| Mean (SD) | 2.10 (1.891) |
| Median | 1.33 |
| Range | (0.9; 6.1) |

Note:
MRD status result based on next-generation sequencing (NGS) or next-generation flow (NGF).
Note:
MRD results were based on either central or local lab assessments.

TABLE 12
Summary of Diagnostic or Baseline Bone Marrow Aspirate
Sample Calibration Rate Based on ClonoSEQ V2.0 Assay;
Cohort D All Treated Analysis Set (Study 68284528MMY2003)

| | Total | |
|---|---|---|
| Analysis set: all treated | 17 | |
| Subjects with sample for testing | 17 | (100.0%) |
| Subjects with calibration success ^{a, b} | 8 | (47.1%) |
| Subjects with calibration failure ^{a, c} | 8 | (47.1%) |
| Subjects with unsuccessful assay run ^{a, d} | 1 | (5.9%) |

| | |
|---|---|
| Subjects without sample for testing | 0 |

^{a} Percentages calculated with the number of subjects with sample for testing as denominator.
^{b} Subjects with multiple baseline samples, of which at least one successfully calibrated, are counted as calibration success only.
^{c} Sample with no clone identified.
^{d} Sample failed QC or not enough DNA.

TABLE 13
Overall Best Response Based on Computerized Algorithm Assessment;
Cohort D All Treated Analysis Set (Study 68284528MMY2003)

| | Total | | |
|---|---|---|---|
| | n (%) | | 95% CI for % |
| Analysis set: all treated | 17 | | |
| Best response | | | |
| Stringent complete response (sCR) | 15 | (88.2%) | (63.6%, 98.5%) |
| MRD-negative CR/sCR ^{a} | 13 | (76.5%) | (50.1%, 93.2%) |
| Complete response (CR) | 1 | (5.9%) | (0.1%, 28.7%) |
| Very good partial response (VGPR) | 0 | | (NE, NE) |
| Partial response (PR) | 0 | | (NE, NE) |
| Minimal response (MR) | 0 | | (NE, NE) |
| Stable disease (SD) | 0 | | (NE, NE) |
| Progressive disease (PD) | 0 | | (NE, NE) |
| Not evaluable | 1 | (5.9%) | (0.1%, 28.7%) |
| Overall response (sCR + CR + | 16 | (94.1%) | (71.3%, 99.9%) |
| VGPR + PR) | | | |
| Clinical benefit (Overall | 16 | (94.1%) | (71.3%, 99.9%) |
| response + MR) | | | |
| VGPR or better (sCR + CR + | 16 | (94.1%) | (71.3%, 99.9%) |
| VGPR) | | | |
| CR or better (sCR + CR) | 16 | (94.1%) | (71.3%, 99.9%) |

Key: CI = confidence interval, NE = Not estimable.
^{a} MRD-negative CR/sCR. Only MRD assessments (10⁻⁵ testing threshold) within 3 months of achieving CR/sCR until death/progression/subsequent therapy (exclusive) are considered.
Note:
Response was assessed by Computerized Algorithm based on International Myeloma Working Group (IMWG) consensus criteria (2016).
Note:
Percentages are calculated with the number of subjects in the all treated analysis set as denominator.
Note:
Exact 95% confidence intervals are provided.
Note:
MRD status result based on next-generation sequencing (NGS) or next-generation flow (NGF).
Note:
MRD results were based on either central or local lab assessments.

TABLE 14
Duration of Response Based on Computerized Algorithm
Assessment; Responders Cohort D in All Treated
Analysis Set (Study 68284528MMY2003)

| | Total | |
|---|---|---|
| Analysis set: responders in | 16 | |
| all treated | | |
| Duration of response | | |
| Number of events (%) | 0 | |
| Number of censored (%) | 16 | (100.0%) |
| Kaplan-Meier estimate (months) | | |
| 25% quantile (95% CI) | NE | (NE, NE) |
| Median (95% CI) | NE | (NE, NE) |
| 75% quantile (95% CI) | NE | (NE, NE) |
| 6-month event-free rate % (95% CI) | 100.0 | (100.0, 100.0) |
| 9-month event-free rate % (95% CI) | 100.0 | (100.0, 100.0) |
| 12-month event-free rate % (95% CI) | 100.0 | (100.0, 100.0) |

Keys: CI = confidence interval, NE = Not estimable.

TABLE 15
Descriptive Summaries for Time to Response Based on Computerized
Algorithm Assessment; Cohort D Responders in All Treated
Analysis Set (Study 68284528MMY2003)

| | Total | |
|---|---|---|
| Analysis set: responders in all treated | 16 | |
| Time to first response^{a} (months) | | |
| N | 16 | |
| Mean (SD) | 3.07 | (3.481) |
| Median | 1.30 | |
| Range | (0.9; 12.5) | |
| Time to best response (months) | | |
| N | 16 | |
| Mean (SD) | 4.02 | (4.021) |
| Median | 1.89 | |
| Range | (0.9; 12.5) | |
| Time to CR or better (months) | | |
| N | 16 | |
| Mean (SD) | 3.30 | (3.448) |

| | |
|---|---|
| Median | 1.72 |
| Range | (0.9; 12.5) |

Key: CR = complete response
^{a}Response PR or better

TABLE 16
Progression-Free Survival Based on Computerized
Algorithm Assessment; Cohort D All Treated
Analysis Set (Study 68284528MMY2003)

| | Total | |
|---|---|---|
| Analysis set: all treated | 17 | |
| Progression-free survival | | |
| Number of events (%) | 1 | (5.9%) |
| Number of censored (%) | 16 | (94.1%) |
| Kaplan-Meier estimate (months) | | |
| 25% quantile (95% CI) | NE | (10.91, NE) |
| Median (95% CI) | NE | (NE, NE) |
| 75% quantile (95% CI) | NE | (NE, NE) |
| 6-month progression-free | 100.0 | (100.0, 100.0) |
| survival rate % (95% CI) | | |
| 9-month progression-free | 100.0 | (100.0, 100.0) |
| survival rate % (95% CI) | | |
| 12-month progression-free | 93.8 | (63.2, 99.1) |
| survival rate % (95% CI) | | |
| 18-month progression-free | 93.8 | (63.2, 99.1) |
| survival rate % (95% CI) | | |

Key: CI = confidence interval, NE = Not estimable.

TABLE 17
Overall Survival; Cohort D All Treated
Analysis Set (Study 68284528MMY2003)

| | Total | |
|---|---|---|
| Analysis set: all treated | 17 | |
| Overall survival | | |
| Number of events (%) | 1 | (5.9%) |
| Number of censored (%) | 16 | (94.1%) |
| Kaplan-Meier estimate (months) | | |
| 25% quantile (95% CI) | NE | (10.91, NE) |
| Median (95% CI) | NE | (NE, NE) |
| 75% quantile (95% CI) | NE | (NE, NE) |
| 6-month overall survival | 100.0 | (100.0, 100.0) |
| rate % (95% CI) | | |
| 9-month overall survival | 100.0 | (100.0, 100.0) |
| rate % (95% CI) | | |
| 12-month overall survival | 93.8 | (63.2, 99.1) |
| rate % (95% CI) | | |
| 18-month overall survival | 93.8 | (63.2, 99.1) |
| rate % (95% CI) | | |

Key: CI = confidence interval, NE = Not estimable.

TABLE 18
Summary of JNJ-68284528 Infusion; Cohort D All
Treated Analysis Set (Study 68284528MMY2003)

| | Total | |
|---|---|---|
| Analysis set: all treated | 17 | |
| Time since initial apheresis to JNJ-68284528 | | |
| Infusion (days) | | |
| N | 17 | |
| Mean (SD) | 86.8 | (25.25) |
| Median | 85.0 | |
| Range | (49; 131) | |
| Time from apheresis to JNJ-68284528 | | |
| Infusion (days)^{a} | | |
| N | 17 | |
| Mean (SD) | 82.2 | (22.70) |
| Median | 78.0 | |
| Range | (49; 119) | |
| Receipt-to-release (R2R)^{b} days | | |
| N | 17 | |
| Mean (SD) | 42.8 | (13.38) |
| Median | 38.0 | |
| Range | (27; 68) | |
| Duration of JNJ-68284528 infusion (minutes) | | |
| N | 17 | |
| Mean (SD) | 18.8 | (7.33) |
| Median | 18.0 | |
| Range | (8; 35) | |
| Total volume infused (mL) | | |
| N | 17 | |
| Mean (SD) | 69.4 | (22.49) |
| Median | 70.0 | |
| Range | (30; 140) | |
| Total CAR-positive viable T cells | | |
| infused (×10⁶ cells) | | |
| N | 17 | |
| Mean (SD) | 56.53 | (14.344) |
| Median | 54.32 | |
| Range | (28.4; 84.5) | |
| JNJ-68284528 dose formulated | | |
| (×10⁶ cells/kg)^{c} | | |
| N | 17 | |
| Mean (SD) | 0.671 | (0.0772) |
| Median | 0.700 | |
| Range | (0.600; 0.800) | |
| JNJ-68284528 dose administered | | |
| (×10⁶ cells/kg)^{d} | | |
| N | 17 | |
| Mean (SD) | 0.677 | (0.0836) |

| | |
|---|---|
| Median | 0.683 |
| Range | (0.564; 0.838) |

^{a}The apheresis that resulted in complete manufacturing of JNJ-68284528 is used if there are multiple apheresis attempts including retreatment.
^{b}R2R is calculated from the day after the receipt of leukapheresis material at the Manufacturing Facility (MFG) up to, and inclusive of the day on which the CAR-T product is released for shipment to the Clinical Trial Site.
^{c}CAR-positive viable T cells based on weight at apheresis.
^{d}CAR-positive viable T cells based on the latest weight on or within 5 days prior to JNJ-68284528 infusion.
Note:
Duration of infusion includes both actual infusion time and interruption time, if any.

TABLE 19
Summary of Lenalidomide Maintenance Therapy; Cohort
D All Treated Analysis Set (Study 68284528MMY2003).

| | Total | |
|---|---|---|
| Analysis set: all treated | 17 | |
| Study day of Lenalidomide start | | |
| relative to JNJ-68284528 | | |
| N | 12 | |
| Mean (SD) | 85.1 | (71.75) |
| Median | 51.0 | |
| Range | (21; 214) | |
| Duration of Lenalidomide (days) | | |
| N | 12 | |
| Mean (SD) | 426.0 | (207.00) |
| Median | 426.5 | |
| Range | (70; 716) | |
| Number of cycles received | | |
| N | 12 | |
| Mean (SD) | 15.0 | (7.15) |
| Median | 15.0 | |
| Range | (3; 26) | |
| Overall relative dose intensity (%)^{a} | | |
| N | 12 | |
| Mean (SD) | 90.10 | (11.001) |
| Median | 93.39 | |
| Range | (67.9; 100.0) | |
| Cycles 1-3 relative dose intensity (%)^{a} | | |
| N | 12 | |
| Mean (SD) | 92.18 | (13.454) |
| Median | 100.00 | |
| Range | (62.5; 100.0) | |
| Cycles 4-6 relative dose intensity (%)^{a} | | |
| N | 11 | |
| Mean (SD) | 90.55 | (13.001) |
| Median | 100.00 | |
| Range | (65.4; 100.0) | |
| Cycles 7-9 relative dose intensity (%)^{a} | | |
| N | 10 | |
| Mean (SD) | 93.61 | (8.959) |
| Median | 98.28 | |
| Range | (75.0; 100.0) | |
| Cycles 10-12 relative dose intensity (%)^{a} | | |
| N | 10 | |
| Mean (SD) | 81.41 | (30.681) |
| Median | 94.64 | |
| Range | (0.0; 100.0) | |
| Cycles 13-15 relative dose intensity (%)^{a} | | |
| N | 9 | |
| Mean (SD) | 93.24 | (12.278) |
| Median | 98.00 | |
| Range | (61.9; 100.0) | |
| Cycles 16-18 relative dose intensity (%)^{a} | | |
| N | 5 | |
| Mean (SD) | 72.98 | (43.537) |
| Median | 100.00 | |
| Range | (0.0; 100.0) | |
| Cycles 19-21 relative dose intensity (%)^{a} | | |
| N | 4 | |
| Mean (SD) | 91.07 | (11.845) |
| Median | 94.64 | |
| Range | (75.0; 100.0) | |
| Cycles 22-24 relative dose intensity (%)^{a} | | |
| N | 3 | |
| Mean (SD) | 96.05 | (6.850) |
| Median | 100.00 | |
| Range | (88.1; 100.0) | |
| Cycles ≥25 relative dose intensity (%)^{a} | | |
| N | 1 | |
| Mean (SD) | 100.00 | (—) |

| | |
|---|---|
| Median | 100.00 |
| Range | (100.0; 100.0) |
| Subjects ongoing Lenalidomide maintenance | 7 |
| therapy | |

^{a}Relative dose intensity is calculated as the percentage of total dose (mg) received in all relevant cycles divided by the sum of prescribed doses (mg) in those cycles.
Note:
When present, relative dose intensities equal to 0 refer to subjects who did not take the drug in the corresponding cycle(s) by the data cutoff date.

TABLE 20
Overall Summary of Treatment-emergent Adverse Events; Cohort
D All Treated Analysis Set (Study 68284528MMY2003)

| | Total | |
|---|---|---|
| Analysis set: all treated | 17 | |
| Any TEAE | 17 | (100.0%) |
| Study drug-related | 17 | (100.0%) |
| Any serious TEAE | 10 | (58.8%) |
| Study drug-related | 9 | (52.9%) |
| Maximum severity of any TEAE | | |
| Grade 1 | 0 | |
| Grade 2 | 0 | |
| Grade 3 | 3 | (17.6%) |
| Grade 4 | 14 | (82.4%) |

| | |
|---|---|
| Grade 5 | 0 |
| TEAE with outcome death ^{a} | 0 |
| Study drug-related | 0 |

Keys: TEAE = treatment-emergent adverse event.
^{a}TEAE with outcome death on the AE eCRF page.
Note:
Percentages are calculated with the number subjects in the all treated analysis set as denominator.
Note:
Adverse events are graded according to the NCI-CTCAE Version 5.0, with the exception of immune effector cell-associated neurotoxicity (ICANS) and cytokine release syndrome (CRS), which were evaluated according to the ASTCT consensus grading system
Note:
The output includes the diagnosis and symptoms of CRS and ICANS along with other AEs.

TABLE 21
Overall Summary of Treatment-emergent Adverse
Events by Lenalidomide Maintenance; Cohort D
All Treated Analysis Set (Study 68284528MMY2003)

| | Cohort D | | | | | |
|---|---|---|---|---|---|---|
| | Received | | Did not Receive | | | |
| | Lenalidomide | | Lenalidomide | | | |
| | Maintenance | | Maintenance | | Total | |
| Analysis set: all | 12 | | 5 | | 17 | |
| treated | | | | | | |
| Any TEAE | 12 | (100.0%) | 5 | (100.0%) | 17 | (100.0%) |
| Study drug-related | 12 | (100.0%) | 5 | (100.0%) | 17 | (100.0%) |
| Any serious TEAE | 9 | (75.0%) | 1 | (20.0%) | 10 | (58.8%) |
| Study drug-related | 9 | (75.0%) | 0 | | 9 | (52.9%) |
| Maximum severity | | | | | | |
| of any TEAE | | | | | | |
| Grade 1 | 0 | | 0 | | 0 | |
| Grade 2 | 0 | | 0 | | 0 | |
| Grade 3 | 1 | (8.3%) | 2 | (40.0%) | 3 | (17.6%) |
| Grade 4 | 11 | (91.7%) | 3 | (60.0%) | 14 | (82.4%) |
| Grade 5 | 0 | | 0 | | 0 | |
| TEAE with outcome | 0 | | 0 | | 0 | |
| death^{a} | | | | | | |

| | | | |
|---|---|---|---|
| Study drug-related | 0 | 0 | 0 |

Keys: TEAE = treatment-emergent adverse event.
^{a}TEAE with outcome death on the AE eCRF page.
Note:
Percentages are calculated with the number subjects in the all treated analysis set as denominator.
Note:
Adverse events are graded according to the NCI-CTCAE Version 5.0, with the exception of immune effector cell-associated neurotoxicity (ICANS) and cytokine release syndrome (CRS), which were evaluated according to the ASTCT consensus grading system
Note:
The output includes the diagnosis and symptoms of CRS and ICANS along with other AEs.

TABLE 22
Number of Subjects With Treatment-emergent Adverse Events With
Frequency of at Least 10% in Total by System Organ Class,
Preferred Term, and Worst Toxicity Grade of 3 or Higher; Cohort
D All Treated Analysis Set (Study 68284528MMY2003)

| | Cohort D | | | | |
|---|---|---|---|---|---|
| | Total | | Grade 3 or 4 | | Grade 5 |
| Analysis set: all treated | 17 | | | | |
| Total number of subjects | 17 | (100.0%) | 17 | (100.0%) | 0 |
| with TEAE | | | | | |
| MedDRA system organ | | | | | |
| class/preferred term | | | | | |
| Blood and lymphatic system | 17 | (100.0%) | 16 | (94.1%) | 0 |
| disorders | | | | | |
| Neutropenia | 16 | (94.1%) | 14 | (82.4%) | 0 |
| Lymphopenia | 11 | (64.7%) | 10 | (58.8%) | 0 |
| Thrombocytopenia | 8 | (47.1%) | 4 | (23.5%) | 0 |
| Leukopenia | 7 | (41.2%) | 6 | (35.3%) | 0 |
| Anaemia | 5 | (29.4%) | 1 | (5.9%) | 0 |
| Febrile neutropenia | 2 | (11.8%) | 2 | (11.8%) | 0 |
| Immune system disorders | 15 | (88.2%) | 0 | | 0 |
| Cytokine release syndrome | 14 | (82.4%) | 0 | | 0 |
| Hypogammaglobulinaemia | 5 | (29.4%) | 0 | | 0 |
| Gastrointestinal disorders | 14 | (82.4%) | 2 | (11.8%) | 0 |
| Diarrhoea | 9 | (52.9%) | 2 | (11.8%) | 0 |
| Nausea | 4 | (23.5%) | 0 | | 0 |
| Abdominal distension | 2 | (11.8%) | 0 | | 0 |
| Abdominal pain | 2 | (11.8%) | 0 | | 0 |
| Abdominal pain upper | 2 | (11.8%) | 0 | | 0 |
| Constipation | 2 | (11.8%) | 0 | | 0 |
| Dyspepsia | 2 | (11.8%) | 0 | | 0 |
| Vomiting | 2 | (11.8%) | 0 | | 0 |
| Investigations | 13 | (76.5%) | 5 | (29.4%) | 0 |
| Aspartate aminotransferase | 3 | (17.6%) | 1 | (5.9%) | 0 |
| increased | | | | | |
| Alanine aminotransferase | 2 | (11.8%) | 2 | (11.8%) | 0 |
| increased | | | | | |
| Blood lactate dehydrogenase | 2 | (11.8%) | 0 | | 0 |
| increased | | | | | |
| CD4 lymphocytes decreased | 2 | (11.8%) | 2 | (11.8%) | 0 |
| Gamma-glutamyltransferase | 2 | (11.8%) | 0 | | 0 |
| increased | | | | | |
| Serum ferritin decreased | 2 | (11.8%) | 0 | | 0 |
| Infections and infestations | 12 | (70.6%) | 5 | (29.4%) | 0 |
| Upper respiratory tract | 7 | (41.2%) | 0 | | 0 |
| infection | | | | | |
| COVID-19 | 5 | (29.4%) | 0 | | 0 |
| Nasopharyngitis | 4 | (23.5%) | 0 | | 0 |
| Pneumonia | 3 | (17.6%) | 2 | (11.8%) | 0 |
| Respiratory syncytial virus | 2 | (11.8%) | 1 | (5.9%) | 0 |
| infection | | | | | |
| Rhinovirus infection | 2 | (11.8%) | 0 | | 0 |
| Sinusitis | 2 | (11.8%) | 1 | (5.9%) | 0 |
| Musculoskeletal and | 10 | (58.8%) | 1 | (5.9%) | 0 |
| connective tissue disorders | | | | | |
| Myalgia | 4 | (23.5%) | 0 | | |
| Back pain | 2 | (11.8%) | 0 | | 0 |
| Muscle spasms | 2 | (11.8%) | 0 | | 0 |
| Musculoskeletal stiffness | 2 | (11.8%) | 0 | | 0 |
| Pain in extremity | 2 | (11.8%) | 0 | | 0 |
| Respiratory, thoracic and | 10 | (58.8%) | 0 | | 0 |
| mediastinal disorders | | | | | |
| Cough | 5 | (29.4%) | 0 | | 0 |
| Productive cough | 5 | (29.4%) | 0 | | 0 |
| Nasal congestion | 3 | (17.6%) | 0 | | 0 |
| Rhinorrhoea | 3 | (17.6%) | 0 | | 0 |
| Epistaxis | 2 | (11.8%) | 0 | | 0 |
| Oropharyngeal pain | 2 | (11.8%) | 0 | | 0 |
| General disorders and | 9 | (52.9%) | 0 | | 0 |
| administration site conditions | | | | | |
| Fatigue | 4 | (23.5%) | 0 | | 0 |
| Chills | 2 | (11.8%) | 0 | | 0 |
| Malaise | 2 | (11.8%) | 0 | | 0 |
| Pyrexia | 2 | (11.8%) | 0 | | 0 |
| Metabolism and nutrition | 9 | (52.9%) | 2 | (11.8%) | 0 |
| disorders | | | | | |
| Hyperferritinaemia | 3 | (17.6%) | 0 | | 0 |
| Hypoalbuminaemia | 2 | (11.8%) | 0 | | 0 |
| Hypocalcaemia | 2 | (11.8%) | 0 | | 0 |
| Hypomagnesaemia | 2 | (11.8%) | 0 | | 0 |
| Hypophosphataemia | 2 | (11.8%) | 0 | | 0 |
| Nervous system disorders | 8 | (47.1%) | 0 | | 0 |
| Headache | 3 | (17.6%) | 0 | | 0 |
| Dysarthria | 2 | (11.8%) | 0 | | 0 |
| Skin and subcutaneous tissue | 8 | (47.1%) | 0 | | 0 |
| disorders | | | | | |
| Hyperhidrosis | 2 | (11.8%) | 0 | | 0 |
| Pruritus | 2 | (11.8%) | 0 | | 0 |
| Eye disorders | 6 | (35.3%) | 1 | (5.9%) | 0 |
| Cardiac disorders | 3 | (17.6%) | 0 | | 0 |
| Sinus tachycardia | 2 | (11.8%) | 0 | | 0 |
| Ear and labyrinth disorders | 3 | (17.6%) | 0 | | 0 |
| Vascular disorders | 3 | (17.6%) | 2 | (11.8%) | 0 |

Key: TEAE = treatment-emergent adverse event.
Note:
Adverse events are reported using MedDRA 26.0.
Note:
Percentages are calculated with the number subjects in the all treated analysis set as denominator.
Note:
Adverse events are graded according to the NCI-CTCAE Version 5.0, with the exception of immune effector cell-associated neurotoxicity (ICANS) and cytokine release syndrome (CRS), which were evaluated according to the ASTCT consensus grading system.
Note:
The output includes the diagnosis of CRS and ICANS along with other AEs and the symptoms of CRS or ICANS are excluded.

TABLE 23
Number of Subjects With Treatment-emergent Serious Adverse Events
With Frequency of at Least 2% in Total by System Organ Class,
Preferred Term, and Worst Toxicity Grade of 3 or Higher; Cohort
D All Treated Analysis Set (Study 68284528MMY2003)

| | Cohort D | | | | |
|---|---|---|---|---|---|
| | Total | | Grade 3 or 4 | | Grade 5 |
| Analysis set: all treated | 17 | | | | |
| Total number of subjects with | 10 | (58.8%) | 9 | (52.9%) | 0 |
| Serious TEAE | | | | | |
| MedDRA system organ | | | | | |
| class/preferred term | | | | | |
| Infections and infestations | 5 | (29.4%) | 5 | (29.4%) | 0 |
| Pneumonia | 2 | (11.8%) | 2 | (11.8%) | 0 |
| Rhinovirus infection | 2 | (11.8%) | 0 | | 0 |
| COVID-19 pneumonia | 1 | (5.9%) | 1 | (5.9%) | 0 |
| Metapneumovirus infection | 1 | (5.9%) | 0 | | 0 |
| Pneumonia influenzal | 1 | (5.9%) | 1 | (5.9%) | 0 |
| Pneumonia viral | 1 | (5.9%) | 1 | (5.9%) | 0 |
| Respiratory syncytial virus | 1 | (5.9%) | 1 | (5.9%) | 0 |
| infection | | | | | |
| Sinusitis | 1 | (5.9%) | 1 | (5.9%) | 0 |
| Streptococcal sepsis | 1 | (5.9%) | 1 | (5.9%) | 0 |
| Nervous system disorders | 3 | (17.6%) | 0 | | 0 |
| Facial nerve disorder | 1 | (5.9%) | 0 | | 0 |
| Facial paralysis | 1 | (5.9%) | 0 | | 0 |
| Peripheral motor neuropathy | 1 | (5.9%) | 0 | | 0 |
| Blood and lymphatic system | 2 | (11.8%) | 2 | (11.8%) | 0 |
| disorders | | | | | |
| Febrile neutropenia | 1 | (5.9%) | 1 | (5.9%) | 0 |
| Neutropenia | 1 | (5.9%) | 1 | (5.9%) | 0 |
| Immune system disorders | 2 | (11.8%) | 0 | | 0 |
| Cytokine release syndrome | 2 | (11.8%) | 0 | | 0 |
| Eye disorders | 1 | (5.9%) | 1 | (5.9%) | 0 |
| Diplopia | 1 | (5.9%) | 1 | (5.9%) | 0 |
| Gastrointestinal disorders | 1 | (5.9%) | 1 | (5.9%) | 0 |
| Diarrhoea | 1 | (5.9%) | 1 | (5.9%) | 0 |
| Musculoskeletal and connective | 1 | (5.9%) | 1 | (5.9%) | 0 |
| tissue disorders | | | | | |
| Musculoskeletal pain | 1 | (5.9%) | 1 | (5.9%) | 0 |
| Vascular disorders | 1 | (5.9%) | 1 | (5.9%) | 0 |
| Haematoma | 1 | (5.9%) | 1 | (5.9%) | 0 |

Key: TEAE = treatment-emergent adverse event.
Note:
Adverse events are reported using MedDRA 26.0.
Note:
Percentages are calculated with the number subjects in the all treated analysis set as denominator.
Note:
Adverse events are graded according to the NCI-CTCAE Version 5.0, with the exception of immune effector cell-associated neurotoxicity (ICANS) and cytokine release syndrome (CRS), which were evaluated according to the ASTCT consensus grading system.
Note:
The output includes the diagnosis of CRS and ICANS along with other AEs and the symptoms of CRS or ICANS are excluded.

TABLE 24
Listing of Second Primary Malignancies During Study; Cohort D All Treated Analysis Set (Study 68284528MMY2003)

| | | | | | | | | | |
|---|---|---|---|---|---|---|---|---|---|
| | | Study Day | | | | Study | | | |
| | | of AE | | | | Day of | Confirmed | | |
| | | Onset/ | | | | Diagnosis^{a} | by | | Regimen |
| | Preferred | AE | Toxicity | Relationship | | (Stage | Pathology | | for |
| Cohort/ | Term | Duration | Grade/ | to JNJ- | | of | Diagnosis/ | | Systemic |
| Subject ID | [Verbatim] | (days)^{a} | SAE | 68284528^{b} | Outcome | Disease) | Method | Treatment | Therapy |

| | | | | | | | |
|---|---|---|---|---|---|---|---|
| D/ | Myelodysplastic | 353/ | 3/Y | NR | Not | | |
| N05BE10004001 | syndrome | | | | Recovered/ | | |
| | [MYELODYSPLASTIC | | | | Not | | |
| | SYNDROME] | | | | Resolved | | |
| | | | 3/Y | NR | Not | 353 | Y/ |
| | | | | | Recovered/ | (Not | ASPIRATE |
| | | | | | Not | Applicable) | |
| | | | | | Resolved | | |

^{a}Study day of diagnosis is in reference to the initial administration of JNJ-68284528 (Day 1).
^{b}Relationship to JNJ-68284528 is assessed by the investigator.

TABLE 25
Listing of Deaths During Study; Cohort D All Treated Analysis Set (Study 68284528MMY2003)

| | | | | | Death | | | |
|---|---|---|---|---|---|---|---|---|
| | | | Date of | Date of | Date | | | JNJ- |
| | | | Initial | Retreatment | (Study | | Relationship | 68284528 |
| Site | Cohort/ | Age(years)/ | JNJ- | JNJ- | Day of | Cause of | to JNJ- | Dose |
| ID | Subject ID | Sex/Race | 68284528 | 68284528 | Death^{a}) | Death | 68284528 | Administered |

| | | | | | | |
|---|---|---|---|---|---|---|
| US10002 | D/ | 39/F/Black | 20 Jun. | 17 May | PROGRESSIVE | . |
| | N05US10002006 | or African | 2022 | 2023 | DISEASE | |
| | | American | | (332) | | |

^{a}Study day of death is in reference to the initial administration of JNJ-68284528 (Day 1)
^{b}Treatment-emergent adverse event.
Note:
Adverse events are coded using MedDRA 26.0.

TABLE 26
Summary of Treatment-emergent Cytokine Release
Syndrome (CRS) Events; Cohort D All Treated
Analysis Set (Study 68284528MMY2003)

| | Total | |
|---|---|---|
| Analysis set: all treated | 17 | |
| Number of subjects with CRS | 14 | (82.4%) |
| Maximum toxicity grade | | |
| Grade 1 | 13 | (76.5%) |
| Grade 2 | 1 | (5.9%) |
| Grade 3 | 0 | |
| Grade 4 | 0 | |
| Grade 5 | 0 | |
| Time from initial infusion of JNJ-68284528 to | | |
| first onset of CRS (days) | | |
| N | 14 | |
| Mean (SD) | 8.1 | (1.49) |
| Median | 8.0 | |
| Range | (6; 11) | |
| Time to recovery of CRS^{a} (days) | | |
| N | 14 | |
| Mean (SD) | 2.6 | (1.01) |
| Median | 2.5 | |
| Range | (1; 5) | |
| Duration of CRS^{b} (days) | | |
| N | 14 | |
| Mean (SD) | 2.6 | (1.01) |
| Median | 2.5 | |
| Range | (1; 5) | |
| Interquartile range | (2.0; 3.0) | |
| <=7 days | 14 | (100.0%) |
| Number of subjects with supportive measures | 11 | (64.7%) |
| to treat CRS^{c} | | |
| Anti-IL-6 receptor Tocilizumab | 5 | (29.4%) |
| IL-1 receptor antagonist Anakinra | 1 | (5.9%) |
| Corticosteroids | 0 | |
| IV fluids | 1 | (5.9%) |
| Vasopressor used | 0 | |
| Oxygen used | 0 | |
| Blow-by | 0 | |
| Nasal cannula low flow (≤6 L/min) | 0 | |
| Nasal cannula high flow (>6 L/min) | 0 | |
| Face mask | 0 | |
| Non-Rebreather mask | 0 | |
| Venturi mask | 0 | |
| Other | 0 | |
| Positive pressure | 0 | |
| Analgesics/Antiinflammatory | 8 | (47.1%) |
| Antiinfectives | 8 | (47.1%) |
| Antiepileptics | 0 | |
| Other* | 1 | (5.9%) |
| Outcome of CRS | | |
| N | 14 | |
| Recovered or resolved | 14 | (100.0%) |

Key: CRS = Cytokine Release Syndrome.
^{a}Calculated for CRS with outcome recovered/resolved or recovered/resolved with sequelae. Time to recovery of the event is calculated as the recovery date of event − onset date of event + 1, including gap between the AE records, if any.
^{b}Calculated using death date as the end date of CRS for subjects who died from CRS or had ongoing CRS at the time of death from another cause. For subjects who were alive with ongoing CRS, last known alive date was used as the end date of CRS; if last known alive date is not available, data cutoff date was used.
^{c}Supportive measures to treat CRS and CRS symptoms are included.
Note:
Time from initial infusion of JNJ-68284528 to first onset of CRS is calculated as first onset date of CRS − initial infusion date of JNJ-68284528 + 1.
Note:
Percentages are calculated with the number of subjects in the all treated analysis set as denominator, except for the outcome of CRS and duration of CRS for which percentages are calculated with the number of subjects with CRS in the all treated analysis set as denominator.
Note:
CRS evaluated according to the ASTCT consensus grading system (Lee et al 2019).
*Other treatments included Caffeine and Prochlorperazine.

TABLE 27
Summary of Treatment-emergent Immune Effector Cell-Associated
Neurotoxicity (ICANS) With Onset After JNJ-68284528 Infusion;
Cohort D All Treated Analysis Set (Study 68284528MMY2003)

| | Total | |
|---|---|---|
| Analysis set: all treated | 17 | |
| Number of subjects with ICANS | 1 | (5.9%) |
| Maximum toxicity grade | | |
| Grade 1 | 1 | (5.9%) |
| Grade 2 | 0 | |
| Grade 3 | 0 | |
| Grade 4 | 0 | |
| Grade 5 | 0 | |
| Time from initial infusion of JNJ-68284528 | | |
| to first onset of ICANS (days) | | |
| N | 1 | |
| Mean (SD) | 7.0 | (—) |
| Median | 7.0 | |
| Range | (7; 7) | |
| Time to recovery of ICANS^{a} (days) | | |
| N | 1 | |
| Mean (SD) | 1.0 | (—) |
| Median | 1.0 | |
| Range | (1; 1) | |
| Duration of ICANS^{b} (days) | | |
| N | 1 | |
| Mean (SD) | 1.0 | (—) |
| Median | 1.0 | |
| Range | (1; 1) | |
| Number of subjects with treatment for ICANS | 1 | (5.9%) |
| Anti-IL-6 receptor Tocilizumab | 0 | |
| IL-1 receptor antagonist anakinra | 0 | |
| Corticosteroids | 0 | |
| Ceftazidime | 1 | (5.9%) |
| Outcome of ICANS | | |
| N | 1 | |
| Recovered or resolved | 1 | (5.9%) |
| Not recovered or not resolved | 0 | |
| Recovered or resolved with sequelae | 0 | |
| Recovering or resolving | 0 | |
| Fatal | 0 | |
| Unknown | 0 | |
| Concurrent CRS | | |
| Yes | 1* | (5.9%) |

| | |
|---|---|
| No | 0 |
| ICANS prior to CRS | 0 |
| ICANS following CRS | 0 |

Keys: CRS = Cytokine Release Syndrome, ICANS = Immune Effector Cell-Associated Neurotoxicity, TE = treatment-emergent.
*Patient was administered Ciprofloxacin, Paracetamol, and Tocilizumab for the treatment of CRS.
^{a}Calculated for ICANS with outcome resolved/recovered. Time to recovery of the event is calculated as the recovery date of event − onset date of event + 1, including gap between the AE records, if any.
^{b}Calculated using death date as the end date of ICANS for subjects who died from ICANS or had ongoing ICANS at the time of death from another cause. For subjects who were alive with ongoing ICANS, last known alive date was used as the end date of ICANS; if last known alive date is not available, data cutoff date was used.
Note:
ICANS evaluated according to the ASTCT consensus grading system (Lee et al 2019)
Note:
Percentages are calculated with the number of subjects in the all treated analysis set as denominator.
Note:
ICANS and CRS are considered to be concurrent if there is an overlap in the duration of these respective events.

TABLE 28
Summary of Other Neurotoxicities; Cohort D All
Treated Analysis Set (Study 68284528MMY2003)

| | Total | |
|---|---|---|
| Analysis set: all treated | 17 | |
| Number of subjects with other | 6 | (35.3%) |
| neurotoxicities | | |
| Maximum toxicity grade | | |
| Grade 1 | 1 | (5.9%) |
| Grade 2 | 4 | (23.5%) |
| Grade 3 | 1 | (5.9%) |
| Grade 4 | 0 | |
| Grade 5 | 0 | |
| Time from initial infusion of JNJ- | | |
| 68284528 to first onset of other | | |
| neurotoxicities (days) | | |
| N | 6 | |
| Mean (SD) | 20.8 | (3.82) |
| Median | 21.0 | |
| Range | (16; 27) | |
| Time to recovery of other | | |
| neurotoxicities^{a} (days) | | |
| N | 4 | |
| Mean (SD) | 153.0 | (195.54) |
| Median | 70.0 | |
| Range | (29; 443) | |
| Duration of other neurotoxicities^{b} (days) | | |
| N | 6 | |
| Mean (SD) | 254.7 | (303.49) |
| Median | 111.0 | |
| Range | (29; 791) | |
| Outcome | | |
| N | 6 | |
| Recovered/Resolved | 4 | (23.5%) |
| Not Recovered/Not Resolved | 2 | (11.8%) |

^{a}Calculated for other neurotoxicities with outcome recovered/resolved. Time to recovery of the event is calculated as the recovery date of event − onset date of event + 1, including gap between the AE records, if any.
^{b}Calculated using death date as the end date of other neurotoxicities for subjects who died from other neurotoxicities or had ongoing other neurotoxicities at the time of death from another cause. For subjects who were alive with ongoing other neurotoxicities, last known alive date was used as the end date of other neurotoxicities; if last known alive date is not available, data cutoff date was used.
Note:
Percentages are calculated with the number of subjects in the all treated analysis set as denominator.
Note:
Study day is in reference to the initial infusion of JNJ-68284528 (Day 1).

TABLE 29
Listing of Potential Movement and Neurocognitive Treatment-emergent Adverse
Events; Cohort D All Treated Analysis Set (Study 68284528MMY2003)

| | | | | | | | | | |
|---|---|---|---|---|---|---|---|---|---|
| | | Line of Prior | | | | | | | |
| | | Therapy/Year | | | Study | | | | |
| | | Since Diagnosis/ | Worst | | Day | | | | Medication/ |
| | | ISS Staging at | Toxicity | | of AE | Toxicity | | | Procedure |
| | Age | Study Baseline/ | Grade | Preferred | Onset ^{a}/ | Grade of | | | Given [Start - |
| Cohort/ | (years)/ | SPD of EMP at | of | Term/ | AE | Other | | Relationship | End Day of |
| Subject | Sex/ | baseline if present | CRS/ | Reported | Duration | Neurotoxicities/ | | to JNJ- | Medication/ |
| ID | Race | (mm²) | ICANS | Term | (days) | SAE | Outcome | 68284528^{b} | Procedure] |

No Data to Report
Keys: AE = adverse event, EMP = extramedullary plasmacytoma, NR = not related, R = related, SAE = serious adverse event, SPD = sum of product of diameters.
^{a} Study day of AE onset is in reference to the initial administration of JNJ-68284528 (Day 1)
^{b}Relationship to JNJ-68284528 is assessed by the investigator.
Note:
Adverse events are coded using MedDRA 26.0.

TABLE 30
Number of Subjects With Adverse Events of Special Interest;
Cohort D All Treated Analysis Set (Study 68284528MMY2003)

| Cohort D | | |
|---|---|---|
| | Total | ≥Grade 3 |

| | |
|---|---|
| Analysis set: all treated | 17 |

| Number of subjects with adverse | 14 | (82.4%) | 4 | (23.5%) |
|---|---|---|---|---|
| event of special interest* | | | | |
| CRS | 14 | (82.4%) | 0 | |
| CAR-T cell related neurotoxicity | 6 | (35.3%) | 1 | (5.9%) |
| ICANS | 1 | (5.9%) | 0 | |
| Other neurotoxicities | 6 | (35.3%) | 1 | (5.9%) |
| Second primary malignancy | 1 | (5.9%) | 1 | (5.9%) |

Note:
Percentages are calculated with the number subjects in the all treated analysis set as denominator.
Note:
Adverse events are graded according to the NCI-CTCAE Version 5.0, with the exception of immune effector cell-associated neurotoxicity (ICANS) and cytokine release syndrome (CRS), which were evaluated according to the ASTCT consensus grading system.
*Includes the overarching AESI term (e.g. CRS, ICANS) as well as any reported associated symptoms of the AESI.

TABLE 31
Incidences of Prolonged Cytopenias Following Treatment With JNJ-
68284528; Cohort D All Treated Analysis Set (Study 68284528MMY2003)

| | Cohort D (N = 17) | | | | | | |
|---|---|---|---|---|---|---|---|
| | | | Initial Grade 3/4 | | Initial Grade 3/4 | | Occurrence of |
| | | | (%) Not | | (%) Not | | Grade 3/4 |
| | Grade 3/4 (%) | | Recovered^{a} | | Recovered^{a} | | (%) >Day 60 (After |
| | After Day 1 | | to <=Grade 2 | | to <=Grade 2 | | Initial Recovery^{a} |
| | Dosing | | by Day 30 | | by Day 60 | | of Grade 3/4) |
| N | 17 | | 17 | | 17 | | 17 |
| Thrombocytopenia | 5 | (29.4%) | 1 | (5.9%) | 1 | (5.9%) | 0 |
| Neutropenia | 15 | (88.2%) | 1 | (5.9%) | 1 | (5.9%) | 6 (35.3%) |
| Lymphopenia | 17 | (100.0%) | 7 | (41.2%) | 5 | (29.4%) | 2 (11.8%) |

| | | | | | |
|---|---|---|---|---|---|
| Anemia | 1 | (5.9%) | 0 | 0 | 0 |

^{a}The lab result with the worst toxicity grade will be used for a calendar day. Recovery definition: must have 2 consecutive Grade <=2 results from separate days if recovery period <=10 days
Note:
Lab results assessed after Day 1 until Day 100 are included in the analysis
Note:
Grade 3/4 thrombocytopenia: Platelets count <50,000 cells/μL; Grade 3/4 neutropenia: Neutrophil count <1000 cells/μL; Grade 3/4 Lymphopenia: Lymphocytes count <0.5 × 10⁹/L.
Note:
Anemia: Grade 3 - hemoglobin <8 g/dL. Grade 4 not defined by laboratory count per NCI-CTCAE v5.
Note:
Study day is in reference to the initial administration of JNJ-68284528 (Day 1).
Note:
Percentages calculated with the number of treated subjects as denominator

TABLE 32
Summary of Treatment-emergent Infections by System Organ
Class, High Level Group Term, Preferred Term, and Worst
Toxicity Grade of Grade 3 or Higher; Cohort D All Treated
Analysis Set (Study 68284528MMY2003)

| | Cohort D | | | | |
|---|---|---|---|---|---|
| | All Grades | | Grade 3 or 4 | | Grade 5 |
| Analysis set: all treated | 17 | | | | |
| Total number of subjects with | 12 | (70.6%) | 5 | (29.4%) | 0 |
| infection TEAE | | | | | |
| MedDRA system organ class/high | | | | | |
| level group term/preferred term | | | | | |
| Infections and infestations | 12 | (70.6%) | 5 | (29.4%) | 0 |
| Infections - pathogen unspecified | 11 | (64.7%) | 3 | (17.6%) | 0 |
| Upper respiratory tract infection | 7 | (41.2%) | 0 | | 0 |
| Nasopharyngitis | 4 | (23.5%) | 0 | | 0 |
| Pneumonia | 3 | (17.6%) | 2 | (11.8%) | 0 |
| Sinusitis | 2 | (11.8%) | 1 | (5.9%) | 0 |
| Acute sinusitis | 1 | (5.9%) | 0 | | 0 |
| Bronchitis | 1 | (5.9%) | 0 | | 0 |
| Enterocolitis infectious | 1 | (5.9%) | 0 | | 0 |
| Gastroenteritis | 1 | (5.9%) | 0 | | 0 |
| Pharyngitis | 1 | (5.9%) | 0 | | 0 |
| Viral infectious disorders | 10 | (58.8%) | 3 | (17.6%) | 0 |
| COVID-19 | 5 | (29.4%) | 0 | | 0 |
| Respiratory syncytial virus | 2 | (11.8%) | 1 | (5.9%) | 0 |
| infection | | | | | |
| Rhinovirus infection | 2 | (11.8%) | 0 | | 0 |
| COVID-19 pneumonia | 1 | (5.9%) | 1 | (5.9%) | 0 |
| Herpes zoster | 1 | (5.9%) | 0 | | 0 |
| Influenza | 1 | (5.9%) | 0 | | 0 |
| Metapneumovirus infection | 1 | (5.9%) | 0 | | 0 |
| Oral herpes | 1 | (5.9%) | 0 | | 0 |
| Pneumonia influenzal | 1 | (5.9%) | 1 | (5.9%) | 0 |
| Pneumonia viral | 1 | (5.9%) | 1 | (5.9%) | 0 |
| Bacterial infectious disorders | 3 | (17.6%) | 1 | (5.9%) | 0 |
| Campylobacter infection | 1 | (5.9%) | 0 | | 0 |
| Enterococcal infection | 1 | (5.9%) | 0 | | 0 |
| Respiratory tract infection | 1 | (5.9%) | 0 | | 0 |
| bacterial | | | | | |
| Streptococcal sepsis | 1 | (5.9%) | 1 | (5.9%) | 0 |

| | | | | |
|---|---|---|---|---|
| Fungal infectious disorders | 3 | (17.6%) | 0 | 0 |
| Aspergillus infection | 1 | (5.9%) | 0 | 0 |
| Candida infection | 1 | (5.9%) | 0 | 0 |
| Fungal foot infection | 1 | (5.9%) | 0 | 0 |
| Tongue fungal infection | 1 | (5.9%) | 0 | 0 |

Note:
Percentages calculated with the number of subjects in all treated analysis set as denominator
Note:
Adverse events were coded using MedDRA 26.0.
Key: TEAE = treatment-emergent adverse event.

TABLE 33
Criteria for Response to Multiple Myeloma Treatment

| Response | Response Criteria |
|---|---|
| Stringent | CR as defined below, plus |
| complete | Normal FLC ratio, and |
| response | Absence of clonal plasma cells (PCs) by immunohistochemistry or 2- |
| (sCR) | to 4-color flow cytometry |
| Complete | Negative immunofixation of serum and urine, and |
| response | Disappearance of any soft tissue plasmacytomas, and |
| (CR)^{a} | <5% PCs in bone marrow |
| | No evidence of initial monoclonal protein isotype(s) on |
| | immunofixation of the serum and urine.^{b} |
| Very good | Serum and urine M-component detectable by immunofixation but not |
| partial | on electrophoresis, or |
| response | ≥90% reduction in serum M-component plus urine M- |
| (VGPR)^{a} | component <100 mg/24 hours |
| Partial | ≥50% reduction of serum M-protein and reduction in 24-hour urinary |
| response | M-protein by ≥90% or to <200 mg/24 hours |
| (PR) | If serum and urine M-protein were not measurable, a decrease ≥50% |
| | in the difference between involved and uninvolved FLC levels was |
| | required in place of the M-protein criteria |
| | If serum and urine M-protein were not measurable, and serum FLC |
| | assay was also not measurable, ≥50% reduction in bone marrow PCs |
| | was required in place of M-protein, provided baseline percentage had |
| | been ≥30% |
| | In addition to the above criteria, if present at baseline, ≥50% |
| | reduction in the size of soft tissue plasmacytomas was also required. |
| Minimal | ≥25% but ≤49% reduction of serum M-protein and reduction in 24- |
| response | hour urine M-protein by 50% to 89% |
| (MR) | In addition to the above criteria, if present at baseline, ≥50% |
| | reduction in the size of soft tissue plasmacytomas was also required. |
| Stable | Not meeting criteria for sCR, CR, VGPR, PR, MR, or progressive |
| disease | disease |
| Progressive | Any one or more of the following criteria: |
| disease^{c} | Increase of 25% from lowest response value in any of the following: |
| | Serum M-component (absolute increase must be ≥0.5 g/dL), |
| | and/or |
| | Urine M-component (absolute increase must be ≥200 mg/24 |
| | hours), and/or |
| | Only in subjects without measurable serum and urine M-protein |
| | levels: the difference between involved and uninvolved FLC |
| | levels (absolute increase must be >10 mg/dL) |
| | Only in subjects without measurable serum and urine M-protein |
| | levels and without measurable disease by FLC levels, bone |
| | marrow PC percentage (absolute increase must be ≥10%). |
| | Appearance of a new lesion(s), ≥50% increase from nadir in sum of |
| | the products of the maximal perpendicular diameters of measured |
| | lesions of >1 lesion, or ≥50% increase in the longest diameter of a |
| | previous lesion >1 cm in short axis |
| | Definite development of new bone lesions or definite increase in the |
| | size of existing bone lesions |
| | ≥50% increase in circulating plasma cells (minimum of 200 cells per |
| | μL) if this was the only measure of disease |

^{a}Clarifications to the criteria for coding CR and VGPR in subjects in whom the only measurable disease is by serum FLC levels: CR in such subjects indicates a normal FLC ratio of 0.26 to 1.65 in addition to CR criteria listed above. VGPR in such subjects requires a ≥90% decrease in the difference between involved and uninvolved FLC levels. For patients achieving very good partial response by other criteria, a soft tissue plasmacytoma must decrease by more than 90% in the sum of the maximal perpendicular diameter (SPD) compared with baseline.
^{b}In some cases it is possible that the original M protein light-chain isotype is still detected on immunofixation but the accompanying heavy-chain component has disappeared; this would not be considered as a CR even though the heavy-chain component is not detectable, since it is possible that the clone evolved to one that secreted only light chains. Thus, if a patient has IgA lambda myeloma, then to qualify as CR there should be no IgA detectable on serum or urine immunofixation; if free lambda is detected without IgA, then it must be accompanied by a different heavy chain isotype (IgG, IgM, etc.).
^{c}Clarifications to the criteria for coding progressive disease: bone marrow criteria for progressive disease are to be used only in subjects without measurable disease by M-protein and by FLC levels; “25% increase” refers to M-protein, and FLC, and does not refer to bone lesions, or soft tissue plasmacytomas and the “lowest response value” does not need to be a confirmed value.
Notes:
All response categories (CR, sCR, VGPR, PR, MR, and progressive disease) require 2 consecutive assessments made at any time before the institution of any new therapy; CR, sCR, VGPR, PR, MR, and stable disease categories also require no known evidence of progressive or new bone lesions if radiographic studies were performed. VGPR and CR categories require serum and urine studies regardless of whether disease at baseline was measurable on serum, urine, both, or neither.
Radiographic studies are not required to satisfy these response requirements. Bone marrow assessments need not be confirmed. For progressive disease, serum M-component increases of ≥1 g/dL are sufficient to define relapse if lowest M-component is ≥5 g/dL.
Source: Adapted from Durie (2015) and Rajkumar (2011), Kumar (2016)

TABLE 34
Cytokine Release Syndrome ASTCT Consensus Grading System

| Grade | Toxicity |
|---|---|
| Grade 1 | Fever^{a} (Temperature ≥38°) |
| Grade 2 | Fever^{a} (Temperature ≥38°) with either: |
| | Hypotension not requiring vasopressors |
| | And/or^{c} hypoxia requiring low-flow nasal cannula^{b} or blow-by. |
| Grade 3 | Fever^{a} (Temperature ≥38°) with either: |
| | Hypotension requiring a vasopressor with or without |
| | vasopressin, |
| | And/or^{c} hypoxia requiring high-flow nasal cannula^{b}, facemask, |
| | nonrebreather mask, or Venturi mask. |
| Grade 4 | Fever^{a} (Temperature ≥38°) with either: |
| | hypotension requiring multiple vasopressors (excluding |
| | vasopressin), |
| | And/or^{c} hypoxia requiring positive pressure (e.g., CPAP, |
| | BiPAP, intubation and mechanical ventilation). |
| Grade 5 | Death |

^{a}Fever not attributable to any other cause. In patients who have CRS then receive antipyretics or anticytokine therapy such as tocilizumab or steroids, fever is no longer required to grade subsequent CRS severity. In this case, CRS grading is driven by hypotension and/or hypoxia.
^{b}Low-flow nasal cannula is defined as oxygen delivered at ≤6 L/minute or blow-by oxygen delivery. High-flow nasal cannula is defined as oxygen delivered at >6 L/minute.
^{c}CRS grade is determined by the more severe event: hypotension or hypoxia not attributable to any other cause.
Note:
Organ toxicities associated with CRS may be graded according to CTCAE v5.0 but they do not influence CRS grading.
Source: Lee (2019)

TABLE 35
Immune Effector Cell-associated Neurotoxicity Syndrome
(ICANS) ASTCT Consensus Grading System^{a,b}

| Neurotoxicity | | | | |
|---|---|---|---|---|
| Domain | Grade 1 | Grade 2 | Grade 3 | Grade 4 |
| ICE Score | 7-9 | 3-6 | 0-2 | 0 (patient is |
| | | | | unarousable and |
| | | | | unable to perform |
| | | | | ICE). |
| Depressed | Awakens | Awakens | Awakens only to | Patient is |
| Level of | spontaneously. | to voice. | tactile stimulus. | unarousable or |
| Consciousness | | | | requires vigorous or |
| | | | | repetitive tactile |
| | | | | stimuli |
| | | | | to arouse. Stupor or |
| | | | | coma. |
| Seizure | N/A | N/A | Any clinical seizure, | Life-threatening |
| | | | focal or generalized, | prolonged seizure |
| | | | that | (>5 min); or |
| | | | resolves rapidly; or | Repetitive clinical |
| | | | Non-convulsive | or electrical |
| | | | seizures on EEG that | seizures |
| | | | resolve with | without return to |
| | | | intervention. | baseline in between. |
| Motor | N/A | N/A | N/A | Deep focal motor |
| Findings | | | | weakness such as |
| | | | | hemiparesis or |
| | | | | paraparesis. |
| Raised | N/A | N/A | Focal/local edema | Diffuse cerebral |
| Intracranial | | | on neuroimaging. | edema on |
| Pressure/ | | | | neuroimaging; or |
| Cerebral | | | | Decerebrate or |
| Edema | | | | decorticate |
| | | | | posturing; or |
| | | | | Cranial nerve VI |
| | | | | palsy; or |
| | | | | Papilledema; or |
| | | | | Cushing's triad. |

^{a}Toxicity grading according to Lee et al 2019
^{b}ICANS grade is determined by the most severe event (ICE score, level of consciousness, seizure, motor findings, raised ICP/cerebral edema) not attributable to any other cause.
Note:
all other neurological adverse events (not associated with ICANS) should continue to be graded with CTCAE Version 5.0 during both phases of the study

### SEQUENCES

Ciltacabtagene autoleucel CAR CD8α signal peptide, CD8α. SP amino
acid sequence
SEQ ID NO: 1
MALPVTALLLPLALLLHAARP
Ciltacabtagene autoleucel CAR BCMA binding domain, VHH1 amino
acid sequence
SEQ ID NO: 2
QVKLEESGGGLVQAGRSLRLSCAASEHTFSSHVMGWFRQAPGKERESVAVIGWRDI
STSYADSVKGRFTISRDNAKKTLYLQMNSLKPEDTAVYYCAARRIDAADFDSWGQG
TQVTVSS
Ciltacabtagene autoleucel CAR BCMA binding domain, G4S linker
amino acid sequence
SEQ ID NO: 3
GGGGS
Ciltacabtagene autoleucel CAR BCMA binding domain, VHH2 amino
acid sequence
SEQ ID NO: 4
EVQLVESGGGLVQAGGSLRLSCAASGRTFTMGWFRQAPGKEREFVAAISLSPTLAY
YAESVKGRFTISRDNAKNTVVLQMNSLKPEDTALYYCAADRKSVMSIRPDYWGQG
TQVTVSS
Ciltacabtagene autoleucel CAR CD8α hinge amino acid sequence
SEQ ID NO: 5
TTTPAPRPPTPAPTIASQPLSLRPEACRPAAGGAVHTRGLDFACD
Ciltacabtagene autoleucel CAR CD8α transmembrane amino acid
sequence
SEQ ID NO: 6
IYIWAPLAGTCGVLLLSLVITLYC
Ciltacabtagene autoleucel CAR CD137 Cytoplasmic amino acid sequence
SEQ ID NO: 7
KRGRKKLLYIFKQPFMRPVQTTQEEDGCSCRFPEEEEGGCEL
Ciltacabtagene autoleucel CAR CD3z Cytoplasmic amino acid sequence
SEQ ID NO: 8
RVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQAL
PPR
Ciltacabtagene autoleucel CAR CD8α signal peptide CD8α. SP nucleic
acid sequence
SEQ ID NO: 9
ATGGCTCTGCCCGTCACCGCTCTGCTGCTGCCTCTGGCTCTGCTGCTGCACGCTGC
TCGCCCT
Ciltacabtagene autoleucel CAR BCMA binding domain, VHH1 nucleic
acid sequence
SEQ ID NO: 10
CAGGTCAAACTGGAAGAATCTGGCGGAGGCCTGGTGCAGGCAGGACGGAGCCTG
CGCCTGAGCTGCGCAGCATCCGAGCACACCTTCAGCTCCCACGTGATGGGCTGGT
TTCGGCAGGCCCCAGGCAAGGAGAGAGAGAGCGTGGCCGTGATCGGCTGGAGG
GACATCTCCACATCTTACGCCGATTCCGTGAAGGGCCGGTTCACCATCAGCCGGG
ACAACGCCAAGAAGACACTGTATCTGCAGATGAACAGCCTGAAGCCCGAGGACA
CCGCCGTGTACTATTGCGCAGCAAGGAGAATCGACGCAGCAGACTTTGATTCCTG
GGGCCAGGGCACCCAGGTGACAGTGTCTAGC
Ciltacabtagene autoleucel CAR BCMA binding domain, G4S linker
nucleic acid sequence
SEQ ID NO: 11
GGAGGAGGAGGATCT
Ciltacabtagene autoleucel CAR BCMA binding domain, VHH2 nucleic
acid sequence
SEQ ID NO: 12
GAGGTGCAGCTGGTGGAGAGCGGAGGCGGCCTGGTGCAGGCCGGAGGCTCTCTG
AGGCTGAGCTGTGCAGCATCCGGAAGAACCTTCACAATGGGCTGGTTTAGGCAG
GCACCAGGAAAGGAGAGGGAGTTCGTGGCAGCAATCAGCCTGTCCCCTACCCTG
GCCTACTATGCCGAGAGCGTGAAGGGCAGGTTTACCATCTCCCGCGATAACGCC
AAGAATACAGTGGTGCTGCAGATGAACTCCCTGAAACCTGAGGACACAGCCCTG
TACTATTGTGCCGCCGATCGGAAGAGCGTGATGAGCATTAGACCAGACTATTGG
GGGCAGGGAACACAGGTGACCGTGAGCAGC
Ciltacabtagene autoleucel CAR CD8α hinge nucleic acid sequence
SEQ ID NO: 13
ACCACGACGCCAGCGCCGCGACCACCAACACCGGCGCCCACCATCGCGTCGCAG
CCCCTGTCCCTGCGCCCAGAGGCGTGCCGGCCAGCGGCGGGGGGCGCAGTGCAC
ACGAGGGGGCTGGACTTCGCCTGTGAT
Ciltacabtagene autoleucel CAR CD8α transmembrane nucleic acid
sequence
SEQ ID NO: 14
ATCTACATCTGGGCGCCCTTGGCCGGGACTTGTGGGGTCCTTCTCCTGTCACTGG
TTATCACCCTTTACTGC
Ciltacabtagene autoleucel CAR CD137 Cytoplasmic nucleic acid
sequence
SEQ ID NO: 15
AAACGGGGCAGAAAGAAACTCCTGTATATATTCAAACAACCATTTATGAGACCA
GTACAAACTACTCAAGAGGAAGATGGCTGTAGCTGCCGATTTCCAGAAGAAGAA
GAAGGAGGATGTGAACTG
Ciltacabtagene autoleucel CAR CD3 Cytoplasmic nucleic acid sequence
SEQ ID NO: 16
AGAGTGAAGTTCAGCAGGAGCGCAGACGCCCCCGCGTACCAGCAGGGCCAGAA
CCAGCTCTATAACGAGCTCAATCTAGGACGAAGAGAGGAGTACGATGTTTTGGA
CAAGAGACGTGGCCGGGACCCTGAGATGGGGGGAAAGCCGAGAAGGAAGAACC
CTCAGGAAGGCCTGTACAATGAACTGCAGAAAGATAAGATGGCGGAGGCCTACA
GTGAGATTGGGATGAAAGGCGAGCGCCGGAGGGGCAAGGGGCACGATGGCCTT
TACCAGGGTCTCAGTACAGCCACCAAGGACACCTACGACGCCCTTCACATGCAG
GCCCTGCCCCCTCGCTAA
Ciltacabtagene autoleucel CAR amino acid sequence
SEQ ID NO: 17
MALPVTALLLPLALLLHAARPQVKLEESGGGLVQAGRSLRLSCAASEHTFSSHVMG
WFRQAPGKERESVAVIGWRDISTSYADSVKGRFTISRDNAKKTLYLQMNSLKPEDTA
VYYCAARRIDAADFDSWGQGTQVTVSSGGGGSEVQLVESGGGLVQAGGSLRLSCA
ASGRTFTMGWFRQAPGKEREFVAAISLSPTLAYYAESVKGRFTISRDNAKNTVVLQ
MNSLKPEDTALYYCAADRKSVMSIRPDYWGQGTQVTVSSTSTTTPAPRPPTPAPTIA
SQPLSLRPEACRPAAGGAVHTRGLDFACDIYIWAPLAGTCGVLLLSLVITLYCKRGR
KKLLYIFKQPFMRPVQTTQEEDGCSCRFPEEEEGGCELRVKFSRSADAPAYQQGQNQ
LYNELNLGRREEYDVLDKRRGRDPEMGGKPRRKNPQEGLYNELQKDKMAEAYSEI
GMKGERRRGKGHDGLYQGLSTATKDTYDALHMQALPPR
Ciltacabtagene autoleucel CAR BCMA binding domain, VHH1 CDR1
amino acid sequence
SEQ ID NO: 18
SHVMG
Ciltacabtagene autoleucel CAR BCMA binding domain, VHH1 CDR2
amino acid sequence
SEQ ID NO: 19
VIGWRDISTSYADSVKG
Ciltacabtagene autoleucel CAR BCMA binding domain, VHH1 CDR3
amino acid sequence
SEQ ID NO: 20
ARRIDAADFDS
Ciltacabtagene autoleucel CAR BCMA binding domain, VHH2 CDR1
amino acid sequence
SEQ ID NO: 21
TFTMG
Ciltacabtagene autoleucel CAR BCMA binding domain, VHH2 CDR2
amino acid sequence
SEQ ID NO: 22
AISLSPTLAYYAESVKG
Ciltacabtagene autoleucel CAR BCMA binding domain, VHH2 CDR3
amino acid sequence
SEQ ID NO: 23
ADRKSVMSIRPDY

1. A method of treating a subject with multiple myeloma, wherein the subject has not achieved a complete response after receiving an initial therapy comprising a stem cell transplantation, the method comprising administering to the subject a dose of T cells comprising a chimeric antigen receptor (CAR) comprising:
(a) an extracellular antigen binding domain capable of specifically binding to an epitope of B-cell maturation antigen (BCMA), wherein the extracellular antigen binding domain comprises a first VHH domain and a second VHH domain, and wherein the first VHH domain comprising a CDR1, a CDR2, and a CDR3 as set forth in the VHH domain comprising the amino acid sequence of SEQ ID NO: 2, and the second VHH domain comprising a CDR1, a CDR2, and a CDR3 as set forth in the VHH domain comprising the amino acid sequence of SEQ ID NO: 4,
(b) a transmembrane domain, and
(c) an intracellular signaling domain.

2. The method of claim 1, wherein
(a) the initial therapy further comprises:
(1) 4 to 8 cycles of an induction therapy,
wherein optionally the induction therapy comprises a proteasome inhibitor (PI) and an immunomodulatory drug (IMiD), further wherein optionally
(a) the PI is bortezomib, carfilzomib, or ixazomib, or the IMiD is lenalidomide, pomalidomide or thalidomide,
(b) the induction therapy further comprises an alkylating agent, further wherein optionally the alkylating agent is cyclophosphamide, and/or
(c) the induction therapy further comprises an anti-CD38 antibody; and
(2) a high-dose chemotherapy, wherein optionally the high-dose chemotherapy comprises melphalan; and/or
(b) the stem cell transplantation is autologous or allogenic stem cell transplantation, wherein optionally the stem cell transplantation is autologous stem cell transplantation (ASCT) or tandem ASCT.

3-11. (canceled)

12. The method of claim 1, further comprising administering to the subject a dose of an immunomodulatory drug (IMiD) after administering to the subject the dose of the T cells,
wherein optionally the subject is not refractory to the IMiD administered after the dose of the T cells.

13. (canceled)

14. The method of claim 12, wherein the IMiD is lenalidomide, wherein optionally
(a) the dose of lenalidomide is about 2.5 mg, about 5 mg, about 10 mg, or about 15 mg daily,
(b) the administering of the dose of lenalidomide is once daily,
(c) the administering of the dose of lenalidomide starts between about 21 days to about 214 days after the administering of the dose of the T cells to the subject, further wherein optionally
(i) the administering of the dose of lenalidomide starts at a median of about 51 days or at a mean of about 85.1 days, and/or
(ii) the administering of the dose of lenalidomide continues for between about 70 days to about 716 days, further wherein optionally the administering of the dose of lenalidomide continues for a median of about 426.5 days or at a mean of about 426.0 days,
(d) the administering of the dose of lenalidomide is daily in a cycle of about 28 days, further wherein optionally
(i) the administering of the dose of lenalidomide continues for between about 3 cycles to about 26 cycles, further wherein optionally the administering of the dose of the lenalidomide continues for a median or a mean of about 15 cycles, and/or
(ii) the administering of the dose of the lenalidomide is at a relative dose intensity of between about 67.9% to about 100.0%, further wherein optionally the administering of the dose of the lenalidomide is at a median relative dose intensity of about 93.4%, and/or
(e) the administering of the dose of lenalidomide continues until the later of when the subject is confirmed progressive disease or unacceptable toxicity, or when it has reached 2 years after start of the administering of the dose of lenalidomide.

15-30. (canceled)

31. The method of claim 1, wherein the subject has further received a lymphodepletion therapy at least about 5 to about 7 days prior to the administering of the dose of the T cells,
wherein optionally the lymphodepletion therapy comprises administering cyclophosphamide and fludarabine daily, and wherein optionally the lymphodepletion therapy comprises cyclophosphamide at a concentration of about 300 mg/m² and fludarabine at a concentration of about 30 mg/m² daily for 3 days.

32. (canceled)

33. The method of claim 31, wherein the subject has further received a bridging therapy prior to the lymphodepletion therapy, wherein optionally the bridging therapy comprises at least one cycle of lenalidomide at a dose of 10 mg per day.

34. The method of claim 1, wherein the dose of the T cells is about 0.5-1.0×10⁶ of the T cells/kg of body weight of the subject,
wherein optionally the dose of the T cells is about 0.56-0.84×10⁶ of the T cells/kg of body weight of the subject, further wherein optionally the dose of the T cells is about 0.68×10⁶ of the T cells/kg of body weight of the subject, or
wherein optionally the dose of the T cells is formulated to be about 0.60-0.80×10⁶ of the T cells/kg of body weight of the subject, further wherein optionally the dose of the T cells is formulated to be about 0.70×10⁶ of the T cells/kg of body weight of the subject.

35-38. (canceled)

39. The method of claim 1, wherein the administering of the dose of the T cells is in a single, two, or three infusions.

40. The method of claim 1, wherein the method is effective in obtaining minimal residual disease (MRD) negativity assessed in the bone marrow of the subject after the administering to the subject the dose of the T cells, wherein optionally
(a) the MRD negativity is assessed using next generation sequencing (NGS) or next generation flow (NGF) on bone marrow aspirate DNA of the subject, wherein optionally the MRD negativity is assessed at a sensitivity of 10⁻⁵, and/or
(b) the method is effective in obtaining MRD negativity at about 0.9 month to about 6.1 months after the administering to the subject the dose of the T cells, further wherein optionally
(i) the method is effective in obtaining MRD negativity at a median time of about 1.33 months after the administering to the subject the dose of the T cells, or
(ii) the method is effective in obtaining MRD negativity at a mean time of about 2.10 months after the administering to the subject the dose of the T cells.

41-44. (canceled)

45. The method of claim 40, wherein the method is effective in obtaining the MRD negativity at a rate of between about 44.0% to about 89.7% at a sensitivity of 10⁻⁵, wherein optionally the method is effective in obtaining the MRD negativity at a rate of about 70.6% at a sensitivity of 10⁻⁵.

46. (canceled)

47. The method of claim 40, wherein the method is effective in obtaining the MRD negativity at a rate of between about 51.9% to about 95.7% at a sensitivity of 10⁻⁵, wherein optionally the method is effective in obtaining the MRD negativity at a rate of about 80.0% at a sensitivity of 10⁻⁵.

48. (canceled)

49. The method of claim 1, wherein the method is effective in obtaining at least one response in the subject after the administering to the subject the dose of the T cells, and wherein the at least one response comprises, in order from better to worse:
(i) a stringent complete response;
(ii) a complete response;
(iii) a very good partial response;
(iv) a partial response; or
(v) a minimal response,
wherein optionally
(a) the method is effective in obtaining a first response of any one of a partial response, a very good partial response, a complete response, or a stringent complete response, further wherein optionally the method is effective in obtaining the first response at a time of between about 0.9 months and about 12.5 months after the administering of the dose of the T cells, and further wherein optionally
(i) the method is effective in obtaining the first response at a mean time of about 3.07 months after the administering of the dose of the T cells, or
(ii) the method is effective in obtaining a first response at a median time of about 1.30 months after the administering of the dose of the T cells,
(b) the method is effective in obtaining a best response of any one of a partial response, a very good partial response, a complete response, or a stringent complete response, further wherein optionally the method is effective in obtaining the best response at a time of between about 0.9 months and about 12.5 months after the administering of the dose of the T cells, and further wherein optionally
(i) the method is effective in obtaining the best response at a mean time of about 4.02 months after the administering of the dose of the T cells, or
(ii) the method is effective in obtaining the best response at a median time of about 1.89 months after the administering of the dose of the T cells,
(c) the method is effective in obtaining a complete response or a stringent complete response at a time of between about 0.9 months and about 12.5 months after the administering of the dose of the T cells, further wherein optionally
(i) the method is effective in obtaining the complete response or the stringent complete response at a mean time of about 3.30 months after the administering of the dose of the T cells, or
(ii) the method is effective in obtaining the complete response or the stringent complete response at a median time of about 1.72 months after the administering of the dose of the T cells,
(d) the method is effective in obtaining a complete response or a stringent complete response at a rate of between about 71.3% and about 99.9%, further wherein optionally the method is effective in obtaining the complete response or the stringent complete response at a rate of about 94.1%,
(e) the method is effective in obtaining a very good partial response, a complete response or a stringent complete response at a rate of between about 71.3% and about 99.9%, further wherein optionally the method is effective in obtaining the very good partial response, the complete response or the stringent complete response at a rate of about 94.1%,
(f) the method is effective in obtaining a partial response, a very good partial response, a complete response or a stringent complete response at a rate of between about 71.3% and about 99.9%, further wherein optionally the method is effective in obtaining the partial response, the very good partial response, the complete response or the stringent complete response at a rate of about 94.1%,
(g) the method is effective in obtaining a minimal response, a partial response, a very good partial response, a complete response or a stringent complete response at a rate of between about 71.3% and about 99.9%, further wherein optionally the method is effective in obtaining the minimal response, the partial response, the very good partial response, the complete response or the stringent complete response at a rate of about 94.1%,
(h) the method is effective in obtaining a best response comprising a stringent complete response at a rate of between about 63.6% and about 98.5%, further wherein optionally the method is effective in obtaining the stringent complete response at a rate of about 88.2%,
(i) the method is effective in obtaining a best response comprising a complete response or a stringent complete response and further comprising a MRD negativity at a rate of between about 50.1% and about 93.2%, wherein optionally the method is effective in obtaining the best response at a rate of about 76.5%,
(j) the method is effective in obtaining a best response comprising a complete response at a rate of between about 0.1% and about 28.7%, wherein optionally the method is effective in obtaining the best response at a rate of about 5.9%, or
(k) the method is effective in maintaining the response for at least about 6 months at a rate of about 100%, further wherein optionally the method is effective in maintaining the response for at least about 9 months or about 12 months at a rate of about 100%.

50-75. (canceled)

76. The method of claim 1, wherein the method is effective in obtaining progression-free survival of the subject,
wherein optionally the method is effective in obtaining the progression-free survival at a rate of about 100.0% at a follow-up time of about 6 months or about 9 months, or
wherein optionally the method is effective in obtaining the progression-free survival at a rate of between about 63.2% and about 99.1% at a follow-up time of about 12 months or about 18 months, wherein optionally the method is effective in obtaining the progression-free survival at a rate of about 93.8% at a follow-up time of about 12 months or about 18 months.

77-80. (canceled)

81. The method of claim 1, wherein the method is effective in obtaining an overall survival rate,
wherein optionally the overall survival rate is about 100.0% at a follow-up time of about 6 months or about 9 months, or
wherein optionally the overall survival rate is between about 63.2% and about 99.1% at a follow-up time of about 12 months or about 18 months, wherein optionally the overall survival rate is about 93.8% at a follow-up time of about 12 months or about 18 months.

82-85. (canceled)

86. The method of claim 1, wherein the method further comprises treating the subject for an adverse event after the administering of the dose of the T cells, wherein optionally the method comprises administering a treatment to the subject to alleviate the adverse event.

87. (canceled)

88. The method of claim 86, wherein the adverse event comprises a treatment-emergent adverse event, wherein optionally the treatment-emergent adverse event is a serious treatment-emergent adverse event, wherein optionally
(a) the treatment-emergent adverse event occurs within the later of about 100 days at or after the administering of the dose of the T cells or about 30 days after last dose of lenalidomide, and/or
(b) the severity of the treatment-emergent adverse event is Grade 1, Grade 2, Grade 3, or Grade 4, further wherein optionally
(i) the severity of the treatment-emergent adverse event is Grade 3 or 4, wherein optionally the maximum severity of treatment-emergent adverse is Grade 4,
(ii) the maximum severity of the treatment-emergent adverse event is Grade 3, wherein optionally the maximum severity of the treatment-emergent adverse event of Grade 3 occurs at a rate of about 17.6%, or
(iii) the maximum severity of the treatment-emergent adverse event is Grade 4, wherein optionally the maximum severity of the treatment-emergent adverse event of Grade 4 occurs at a rate of about 82.4%.

89-93. (canceled)

94. The method of claim 88, wherein the treatment-emergent adverse event comprises a blood or lymphatic system disorder, an immune system disorder, a gastrointestinal disorder, an infection, an infestation, a musculoskeletal or connective tissue disorder, a respiratory, thoracic or mediastinal disorder, a general disorder, an administrate site condition, a metabolism or nutrition disorder, a nervous system disorder, a skin or subcutaneous tissue disorder, an eye disorder, a cardiac disorder, an ear or labyrinth disorder, or a vascular disorder, wherein optionally the treatment-emergent adverse event occurs at a rate of at least about 10%.

95. The method of claim 88, wherein the treatment-emergent adverse event comprises neutropenia, lymphopenia, thrombocytopenia, leukopenia, anemia, febrile neutropenia, cytokine release syndrome, hypogammaglobulinaemia, diarrhoea, nausea, abdominal distension, abdominal pain, upper abdominal pain, constipation, dyspepsia, vomiting, increased aspartate aminotransferase, increased alanine aminotransferase, increased blood lactate dehydrogenase, decreased CD4 lymphocytes, increased gamma-glutamyltransferase, decreased serum ferritin, an upper respiratory tract infection, COVID-19, nasopharyngitis, pneumonia, respiratory syncytial virus infection, Rhinovirus infection, sinusitis, myalgia, back pain, muscle spasm, musculoskeletal stiffness, pain in extremity, cough, productive cough, nasal congestion, rhinorrhoea, epistaxis, oropharyngeal pain, fatigue, chill, malaise, pyrexia, hyperferritinaemia, hypoalbuminaemia, hypocalcaemia, hypomagnesaemia, hypophosphataemia, headache, dysarthria, hyperhidrosis, pruritus, or sinus tachycardia, wherein optionally the treatment-emergent adverse event occurs at a rate of at least about 10%.

96. The method of claim 88, wherein the treatment-emergent adverse event is a serious treatment-emergent adverse event that occurs at a rate of about 58.8%, wherein optionally maximum severity of the treatment-emergent adverse event is Grade 3 or Grade 4, wherein further optionally the maximum severity of the treatment-emergent adverse event of Grade 3 or Grade 4 occurs at a rate of about 52.9%,
wherein optionally the serious treatment-emergent adverse event comprises an infection, an infestation, a nervous system disorder, a blood or lymphatic system disorder, an immune system disorder, an eye disorder, a gastrointestinal disorder, a musculoskeletal or connective tissue disorder, or a vascular disorder, wherein optionally the serious treatment-emergent adverse event occurs at a rate of at least about 2%, or
wherein optionally the serious treatment-emergent adverse event comprises pneumonia, Rhinovirus infection, COVID-19 pneumonia, metapneumovirus infection, influenzal pneumonia, viral pneumonia, respiratory syncytial virus infection, sinusitis, streptococcal sepsis, facial nerve disorder, facial paralysis, peripheral motor neuropathy, febrile neutropenia, neutropenia, cytokine release syndrome, diplopia, diarrhoea, musculoskeletal pain, or haematoma, wherein optionally the serious treatment-emergent adverse event occurs at a rate of at least about 2%.

97-98. (canceled)

99. The method of claim 88, wherein the treatment-emergent adverse event comprises cytokine release syndrome (CRS), wherein optionally the CRS occurs at a rate of about 82.4%, wherein optionally:
(1) time to first onset of the CRS ranges from about 6 days to about 11 days after the administering of the dose of the T cells to the subject, wherein further optionally the time to the first onset of the CRS is at a median of about 8.0 days or at a mean of about 8.1 days;
(2) time to recovery of the CRS ranges from about 1 day to about 5 days, wherein further optionally the time to recovery of the CRS is at a median time of about 2.5 days or at a mean time of about 2.6 days;
(3) duration of the CRS ranges from about 1 day to about 5 days or from about 2 days to about 3 days, wherein further optionally the duration of the CRS is at a median time of about 2.5 days or at a mean time of about 2.6 days; or
(4) duration of the CRS of less than about 7 days occurs at a rate of about 100%,
further wherein optionally
(a) the CRS occurs at maximum toxicity grade of Grade 1, Grade 2, Grade 3, or Grade 4, wherein optionally:
(1) the CRS occurs at the maximum toxicity grade of Grade 1 at a rate of about 76.5%; or
(2) the CRS occurs at the maximum toxicity grade of Grade 2 at a rate of about 5.9%,
(b) the treatment comprises an anti-IL-6 receptor, an IL-1 receptor antagonist, a corticosteroid, IV fluids, a vasopressor, oxygen, an analgesic, an anti-inflammatory drug, an antiinfective, an antiepileptic, caffeine, or prochlorperazine, or any combination thereof, wherein optionally:
(1) the anti-IL-6 receptor comprises tocilizumab;
(2) the IL-1 receptor antagonist comprises anakinra; or
(3) oxygen comprises blow-by, nasal cannula low flow at a flow rate of at most about 6 L/min, nasal cannula high flow at a flow rate of at least about 6 L/min, face mask, non-rebreather mask, venturi mask, or positive pressure, or any combination thereof; and/or
(c) the treatment is effective at achieving a recovery or resolution of the CRS, wherein optionally the treatment is effective at achieving a recovery or resolution of the CRS at a rate of about 100%.

100-102. (canceled)

103. The method of claim 88, wherein the treatment-emergent adverse event comprises immune effector cell-associated neurotoxicity (ICANS), wherein optionally the ICANS occurs at a rate of about 5.9%, and wherein optionally:
(1) time to first onset of the ICANS is about 7 days after the administering of the dose of the T cells to the subject, wherein further optionally the time to the first onset of the ICANS is at a median of about 7 days or at a mean of about 7 days;
(2) time to recovery of the ICANS is about 1 day, wherein further optionally the time to recovery of the ICANS is at a median time of about 1 day or at a mean time of about 1 day;
(3) duration of the ICANS is about 1 day, wherein further optionally the duration of the ICANS is at a median time of about 1 day or at a mean time of about 1 day; or
(4) the ICANS occurs concurrently with cytokine release syndrome (CRS) at a rate of about 5.9%,
further wherein optionally
(a) the ICANS occurs at maximum toxicity grade of Grade 1, Grade 2, Grade 3, or Grade 4, wherein optionally the ICANS occurs at maximum toxicity grade of Grade 1 at a rate of about 5.9%,
(b) the treatment comprises an anti-IL-6 receptor, an IL-1 receptor antagonist, a corticosteroid, or ceftazidime, or any combination thereof, wherein optionally:
(1) the anti-IL-6 receptor comprises tocilizumab; or
(2) the IL-1 receptor antagonist comprises anakinra, and/or
(c) the ICANS is recovered, resolved, not recovered, not resolved, recovered with sequelae, resolved with sequelae, recovering, resolving, or unknown, wherein optionally the treatment is effective at achieving a recovery or resolution of the ICANS at a rate of about 100%.

104-106. (canceled)

107. The method of claim 86, wherein the adverse event comprises neurotoxicity, wherein optionally the neurotoxicity occurs at a rate of about 35.3%, wherein optionally:
(1) time to first onset of the neurotoxicity ranges from about 16 days to about 27 days after the administering of the dose of the T cells to the subject, wherein further optionally the time to the first onset of the neurotoxicity is at a median of about 21.0 days or at a mean of about 20.8 days;
(2) time to recovery of the neurotoxicity ranges from about 29 days to about 443 days, wherein further optionally the time to recovery of the neurotoxicity is at a median time of about 70.0 days or at a mean time of about 153.0 days;
(3) duration of the neurotoxicity ranges from about 29 days to about 791 days, wherein further optionally the duration of the neurotoxicity is at a median time of about 111.0 days or at a mean time of about 254.7 days,
further wherein optionally the neurotoxicity is recovered, resolved, not recovered, or not resolved, wherein optionally the neurotoxicity is recovered or resolved at a rate of about 23.5%, and wherein optionally the neurotoxicity is not recovered or not resolved at a rate of about 11.8%.

108. (canceled)

109. The method of claim 86, wherein the adverse event is an adverse event of special interest, wherein optionally:
(1) the adverse event of special interest occurs at a rate of about 82.4%, wherein further optionally the adverse event of special interest occurs at least at Grade 3 at a rate of about 23.5%; or
(2) the adverse event of special interest comprises cytokine release syndrome (CRS), CAR-T cell related neurotoxicity, second primary malignancy, or a movement and neurocognitive treatment-emergent adverse event, or any combination thereof,
further wherein optionally
(a) the adverse event of special interest is CRS, wherein optionally the CRS occurs at a rate of about 82.4%, wherein further optionally the CRS occurs at least at Grade 3 at a rate of about 0%,
(b) the adverse event of special interest is CAR-T cell related neurotoxicity, wherein optionally:
(1) the CAR-T cell related neurotoxicity occurs at a rate of about 35.3%, wherein further optionally the CAR-T cell related neurotoxicity occurs at least at Grade 3 at a rate of about 5.9%; or
(2) the CAR-T cell related neurotoxicity comprises immune effector cell-associated neurotoxicity (ICANS) or other neurotoxicity, wherein further optionally the ICANS occurs at a rate of about 5.9%, wherein further optionally the ICANS occurs at least at Grade 3 at a rate of about 0%, wherein further optionally the other neurotoxicity occurs at a rate of about 35.3%, and wherein further optionally the other neurotoxicity occurs at least at Grade 3 at a rate of about 5.9%,
(c) the adverse event of special interest is second primary malignancy, wherein optionally the second primary malignancy comprises myelodysplastic syndrome, wherein optionally the second primary malignancy occurs at a rate of about 5.9%, wherein further optionally the second primary malignancy occurs at least at Grade 3 at a rate of about 5.9%, or
(d) the adverse event of special interest is a movement and neurocognitive treatment-emergent adverse event, wherein optionally the movement and neurocognitive treatment-emergent adverse event does not occur in the subject.

110-113. (canceled)

114. The method of claim 86, wherein the adverse event comprises prolonged cytopenia, wherein optionally the prolonged cytopenia comprises thrombocytopenia, neutropenia, lymphopenia, or anemia, or any combination thereof, wherein further optionally:
(1) thrombocytopenia occurs at Grade 3 or 4 at a rate of about 29.4% after the administering of the dose of the T cells to the subject, wherein further optionally thrombocytopenia recovers to Grade 2 or less by about 30 days or about 60 days, and wherein further optionally thrombocytopenia reoccurs at Grade 3 or 4 at a rate of about 0%;
(2) neutropenia occurs at Grade 3 or 4 at a rate of about 88.2% after the administering of the dose of the T cells to the subject, wherein further optionally neutropenia recovers to Grade 2 or less by about 30 days or about 60 days, and wherein further optionally neutropenia reoccurs at Grade 3 or 4 at a rate of about 35.3%;
(3) lymphopenia occurs at Grade 3 or 4 at a rate of about 100% after the administering of the dose of the T cells to the subject, wherein further optionally lymphopenia recovers to Grade 2 or less by about 30 days or about 60 days, and wherein further optionally lymphopenia reoccurs at Grade 3 or 4 at a rate of about 11.8%; or
(4) anemia occurs at Grade 3 or 4 at a rate of about 5.9% after the administering of the dose of the T cells to the subject, wherein further optionally anemia recovers to Grade 2 or less by about 30 days or about 60 days, and wherein further optionally anemia reoccurs at Grade 3 or 4 at a rate of about 0%.

115. The method of claim 88, wherein the treatment-emergent adverse event comprises a treatment-emergent infection, wherein optionally:
(1) the treatment-emergent infection occurs at a rate of about 70.6%, wherein further optionally the treatment-emergent infection occurs at Grade 3 or 4 at a rate of about 29.4%;
(2) the treatment-emergent infection comprises an infection, an infestation, a viral infectious disorder, a bacterial infectious disorder, or a fungal infectious disorder, or any combination thereof;
(3) the treatment-emergent infection comprises an upper respiratory tract infection, nasopharyngitis, pneumonia, sinusitis, acute sinusitis, bronchitis, infectious enterocolitis, gastroenteritis, pharyngitis, COVID-19, respiratory syncytial virus infection, Rhinovirus infection, COVID-19 pneumonia, herpes zoster, influenza, metapneumovirus infection, oral herpes, influenzal pneumonia, viral pneumonia, Campylobacter infection, Enterococcal infection, bacterial respiratory tract infection, Streptococcal sepsis, Aspergillus infection, Candida infection, fungal foot infection, or tongue fungal infection, or any combination thereof.

116. The method of claim 1, wherein the first VHH domain comprises a CDR1 comprising the amino acid sequence of SEQ ID NO: 18, a CDR2 comprising the amino acid sequence of SEQ ID NO: 19, a CDR3 comprising the amino acid sequence of SEQ ID NO: 20, and the second VHH domain comprises a CDR1 comprising the amino acid sequence of SEQ ID NO: 21, a CDR2 comprising the amino acid sequence of SEQ ID NO: 22, and a CDR3 comprising the amino acid sequence of SEQ ID NO: 23, wherein optionally the first VHH domain comprises the amino acid sequence of SEQ ID NO: 2 and the second VHH domain comprises the amino acid sequence of SEQ ID NO: 4, wherein optionally the first VHH domain is at the N-terminus of the second VHH domain, or the first VHH domain is at the C-terminus of the second VHH domain, further wherein optionally:
(a) the first VHH domain is linked to the second VHH domain via a linker, wherein optionally the linker comprises the amino acid sequence of SEQ ID NO: 3;
(b) the transmembrane domain is derived from a molecule selected from the group consisting of CD8α, CD4, CD28, CD137, CD80, CD86, CD152 and PD1, wherein optionally the transmembrane domain is derived from CD8α, wherein optionally the transmembrane domain comprises the amino acid sequence of SEQ ID NO: 6;
(c) the intracellular signaling domain comprises a primary intracellular signaling domain of an immune effector cell, wherein optionally the primary intracellular signaling domain is derived from CD3ζ, wherein optionally the primary intracellular signaling domain comprises the amino acid sequence of SEQ ID NO: 8;
(d) the intracellular signaling domain comprises a co-stimulatory signaling domain, wherein optionally the co-stimulatory signaling domain is derived from a co-stimulatory molecule selected from the group consisting of CD27, CD28, CD137, OX40, CD30, CD40, CD3, LFA-1, ICOS, CD2, CD7, LIGHT, NKG2C, B7-H3, ligands of CD83 and any combination thereof, wherein optionally the co-stimulatory signaling domain comprises a cytoplasmic domain of CD137, wherein optionally the co-stimulatory signaling domain comprises the amino acid sequence of SEQ ID NO: 7;
(e) the CAR further comprises a hinge domain located between the C-terminus of the extracellular antigen binding domain and the N-terminus of the transmembrane domain, wherein optionally the hinge domain is derived from CD8α, wherein optionally the hinge domain comprises the amino acid sequence of SEQ ID NO: 5;
(f) the CAR further comprises a signal peptide located at the N-terminus of the polypeptide, wherein optionally the signal peptide is derived from CD8α comprising the amino acid sequence of SEQ ID NO: 1; or
(g) the CAR comprises the amino acid sequence of SEQ ID NO: 17.

117. The method of claim 1, wherein the dose of the T cells is formulated in a composition comprising dimethyl sulfoxide (DMSO), wherein optionally the DMSO is at a concentration of about 5%.

118. A method of treating a subject with multiple myeloma, wherein the subject has not achieved a complete response after receiving an initial therapy comprising (1) 4 to 8 cycles of an induction therapy, (2) a high-dose chemotherapy, and (3) an autologous stem cell transplantation (ASCT), the method comprising:
(1) administering to the subject a dose of T cells comprising a chimeric antigen receptor (CAR) comprising:
(a) an extracellular antigen binding domain capable of specifically binding to an epitope of B-cell maturation antigen (BCMA), wherein the extracellular antigen binding domain comprises a first VHH domain comprising the amino acid sequence of SEQ ID NO: 2 and a second VHH domain comprising the amino acid sequence of SEQ ID NO: 4,
(b) a transmembrane domain comprising the amino acid sequence of SEQ ID NO: 6 and
(c) an intracellular signaling domain comprising the amino acid sequence of SEQ ID NO: 8, and
(2) optionally administering to the subject a dose of an immunomodulatory drug (IMiD) after administering to the subject the dose of the T cells.

119. A method of treating a subject with multiple myeloma, wherein the subject has not achieved a complete response after receiving an initial therapy comprising (1) 4 to 8 cycles of an induction therapy, (2) a high-dose chemotherapy, and (3) an autologous stem cell transplantation (ASCT), the method comprising:
(1) administering to the subject a dose of T cells comprising a chimeric antigen receptor (CAR) comprising the amino acid sequence of SEQ ID NO: 17, and
(2) optionally administering to the subject a dose of an immunomodulatory drug (IMiD) after administering to the subject the dose of the T cells.
